(19) United States
(12) Patent Application Publication
(10) Pub. No.: US 2012/0138484 A1
(43) Pub. Date: Jun. 7, 2012

(54) Analyte Sensors Comprising Thickeners, Enzyme Stabilizers and Osmium Boronates

(75) Inventors: Balasubrahmanya S. Bommakanti, Pleasanton, CA (US); Udo Hoss, Castro Valley, CA (US); Gary Sandhu, Fairfield, CA (US)

(73) Assignee: Abbott Diabetes Care Inc.

(21) Appl. No.: 13/298,020

(22) Filed: Nov. 16, 2011

Related U.S. Application Data
(60) Provisional application No. 61/421,994, filed on Dec. 10, 2010.
(60) Provisional application No. 61/419,620, filed on Dec. 3, 2010.

(51) Int. Cl. G01N 33/50; B05D 5/00; G01N 27/26

(52) U.S. Cl. 205/777.5; 204/403.14; 427/2.11

(57) ABSTRACT

Embodiments of the present disclosure relate to analyte determining methods and devices (e.g., electrochemical analyte monitoring systems) that have improved uniformity of distribution and/or improved sensitivity and/or reduced sensitivity variation of the sensing layer by inclusion of a thickener and/or an enzyme stabilizer in the sensing layer. Embodiments of the present disclosure also relate to transition metal complexes having at least one pyridine boronic acid ligand are also described. Aspects of the disclosure include transition metal complexes chemically bonded to a polymer, such as a sensing layer polymer. The sensing layer is disposed on a working electrode of in vivo and/or in vitro analyte sensors, e.g., continuous and/or automatic in vivo monitoring using analyte sensors and/or test strips. Also provided are systems and methods of using the, for example electrochemical, analyte sensors in analyte monitoring.

### CROSS-REFERENCE TO RELATED APPLICATIONS

This application claims the benefit of U.S. Provisional Application No. 61/421,994, filed on Dec. 10, 2010, and U.S. Provisional Application No. 61/419,620, filed on Dec. 3, 2010, the disclosures of each of which are herein incorporated by reference in their entirety.

### INTRODUCTION

Enzyme based electrochemical sensors are widely used in the detection of analytes in clinical, environmental, agricultural and biotechnological applications. Analytes that can be measured in clinical assays of fluids of the human body include, for example, glucose, lactate, cholesterol, bilirubin and amino acids. Levels of these analytes in biological fluids, such as blood, are important for the diagnosis and the monitoring of diseases.

Electrochemical assays are typically performed in cells with two or three electrodes, including at least one measuring or working electrode and one reference electrode. In three electrode systems, the third electrode is a counter-electrode. In two electrode systems, the reference electrode also serves as the counter-electrode. The electrodes are connected through a circuit, such as a potentiostat. The measuring or working electrode is a non-corroding carbon or metal conductor. Upon passage of a current through the working electrode, a redox enzyme is electrooxidized or electroreduced, The enzyme is specific to the analyte to be detected, or to a product of the analyte. The turnover rate of the enzyme is typically related (preferably, but not necessarily, linearly) to the concentration of the analyte itself, or to its product, in the test solution.

The electrooxidation or electroreduction of the enzyme is often facilitated by the presence of a redox mediator in the solution or on the electrode. The redox mediator assists in the electrical communication between the working electrode and the enzyme. The redox mediator can be dissolved in the fluid to be analyzed, which is in electrolytic contact with the electrodes, or can be applied within a coating on the working electrode in electrolytic contact with the analyzed solution. The coating is preferably not soluble in water, though it may swell in water. Useful devices can be made, for example, by coating an electrode with a film that includes a redox mediator and an enzyme where the enzyme is catalytically specific to the desired analyte, or its product. In contrast to a coated redox mediator, a diffusional redox mediator, which can be soluble or insoluble in water, functions by shuttling electrons between, for example, the enzyme and the electrode. In any case, when the substrate of the enzyme is electrooxidized, the redox mediator transports electrons from the substrate-reduced enzyme to the electrode; when the substrate is electroreduced, the redox mediator transports electrons from the electrode to the substrate-oxidized enzyme.

In many instances it is desirable or necessary to regularly monitor the concentration of particular constituents in a fluid. A number of systems are available that analyze the constituents of bodily fluids such as blood, urine and saliva. Examples of such systems conveniently monitor the level of particular medically significant fluid constituents, such as, for example, cholesterol, ketones, vitamins, proteins, and various metabolites or blood sugars, such as glucose. Diagnosis and management of patients suffering from diabetes mellitus, a disorder of the pancreas where insufficient production of insulin prevents normal regulation of blood sugar levels, requires carefully monitoring of blood glucose levels on a daily basis. A number of systems that allow individuals to easily monitor their blood glucose are currently available. Such systems include electrochemical biosensors, including those that comprise a glucose sensor that is adapted for insertion into a subcutaneous site within the body for the continuous monitoring of glucose levels in bodily fluid of the subcutaneous site (see for example, U.S. Pat. No. 6,175,752 to Say et al).

A person may obtain a blood sample by withdrawing blood from a blood source in his or her body, such as a vein, using a needle and syringe, for example, or by lancing a portion of his or her skin, using a lancing device, for example, to make blood available external to the skin, to obtain the necessary sample volume for in vitro testing. The person may then apply the fresh blood sample to a test strip, whereupon suitable detection methods, such as calorimetric, electrochemical, or photometric detection methods, for example, may be used to determine the person's actual blood glucose level. The foregoing procedure provides a blood glucose concentration for a particular or discrete point in time, and thus, must be repeated periodically, in order to monitor blood glucose over a longer period.

In addition to the discrete or periodic, or in vitro, blood glucose-monitoring systems described above, at least partially implantable, or in vivo, blood glucose-monitoring systems, which are constructed to provide continuous in vivo measurement of an individual's blood glucose concentration, have been described and developed.

Such analyte monitoring devices are constructed to provide for continuous or automatic monitoring of analytes, such as glucose, in the blood stream or interstitial fluid. Such devices include electrochemical sensors, at least a portion of which are operably positioned in a blood vessel or in the subcutaneous tissue of a user.

While continuous glucose monitoring is desirable, there are several challenges associated with optimizing manufacture protocols to improve yield and uniformity of the sensing layer of the biosensors constructed for in vivo use. Accordingly, further development of manufacturing techniques and methods, as well as analyte-monitoring devices, systems, or kits employing the same, is desirable.

### SUMMARY

Embodiments of the present disclosure relate to analyte determining methods and devices (e.g., electrochemical analyte monitoring systems) that have improved uniformity of distribution and/or improved sensitivity and/or reduced sensitivity variation of the sensing layer by inclusion of a thickener and/or an enzyme stabilizer in the sensing layer. Embodiments of the present disclosure also relate to transition metal complexes having at least one pyridine boronic acid ligand are also described. Aspects of the disclosure include transition metal complexes chemically bonded to a polymer, such as a sensing layer polymer. The sensing layer is disposed on a working electrode of in vivo and/or in vitro analyte sensors, e.g., continuous and/or automatic in vivo monitoring using analyte sensors and/or test strips. Also provided are systems and methods of using the, for example electrochemical, analyte sensors in analyte monitoring.

In this section, embodiments of the present disclosure that include a thickener and/or an enzyme stabilizer are summarized first below. Subsequently, embodiments of the present disclosure that include transition metal complexes are summarized.

Aspects of the present disclosure include an analyte sensor. The analyte sensor includes a working electrode, a counter electrode, and a sensing layer disposed on the working electrode, where the sensing layer includes an analyte-responsive enzyme, a redox mediator and a thickener including one or more of urea, a urea derivative, urethane, a urethane derivative, a polyvinyl pyrrolidone polymer and a polyvinyl pyrrolidone polymer derivative.

In certain embodiments, at least a portion of the analyte sensor is adapted to be subcutaneously positioned in a subject.

In some embodiments, the sensing layer has an arcuate profile as measured using a profilometer.

In certain cases, the analyte sensor has a sensitivity that is 90% or more of its initial sensitivity after 14 days or more.

In some instances, the analyte sensor has an average response time that is 35% or more lower than the average response time of a sensor that does not include a thickener.

In certain embodiments, the analyte-responsive enzyme and the redox mediator are distributed throughout the sensing layer.

In certain cases, the analyte sensor further includes a membrane disposed over the sensing layer, where the membrane limits flux of analyte to the sensing layer. In some instances, only the sensing layer includes the thickener.

In certain embodiments, the analyte sensor is a glucose sensor. In some of these embodiments, the analyte-responsive enzyme includes a glucose-responsive enzyme. In some cases, the glucose-responsive enzyme includes glucose oxidase. In some instances, the redox mediator includes a ruthenium-containing complex or an osmium-containing complex.

In certain embodiments, the analyte sensor is an in vivo sensor. In other embodiments, the analyte sensor is an in vitro sensor.

Aspects of the present disclosure also include a method for monitoring a level of an analyte in a subject. The method includes positioning at least a portion of an analyte sensor into skin of a subject, and determining a level of an analyte over a period of time from signals generated by the analyte sensor, where the determining over a period of time provides for monitoring the level of the analyte in the subject. The analyte sensor includes a working electrode, a counter electrode, and a sensing layer disposed on the working electrode, where the sensing layer includes an analyte-responsive enzyme, a redox mediator and a thickener including one or more of urea, a urea derivative, urethane, a urethane derivative, a polyvinyl pyrrolidone polymer and a polyvinyl pyrrolidone polymer derivative.

In certain embodiments, the sensing layer has an arcuate profile as measured using a profilometer.

In some instances, the analyte sensor has a sensitivity that is 90% or more of its initial sensitivity after 14 days or more.

In certain cases, the analyte sensor has an average response time that is 35% or more lower than the average response time of a sensor that does not include a thickener.

In certain embodiments, the analyte-responsive enzyme and the redox mediator are distributed throughout the sensing layer.

In certain instances, the analyte sensor further includes a membrane disposed over the sensing layer, where the membrane limits flux of the analyte to the sensing layer. In some of these embodiments, only the sensing layer comprises the thickener.

In certain cases, the analyte sensor is a glucose sensor. In some instances, the analyte-responsive enzyme comprises a glucose-responsive enzyme. In certain instances, the glucose-responsive enzyme comprises glucose oxidase. In some embodiments, the redox mediator includes a ruthenium-containing complex or an osmium-containing complex.

Aspects of the present disclosure also include a method for monitoring a level of an analyte using an analyte monitoring system. The method includes inserting at least a portion of an analyte sensor into skin of a patient, attaching an analyte sensor control unit to the skin of the patient, coupling a plurality of conductive contacts of the analyte sensor control unit to a plurality of contact pads of the analyte sensor, collecting data, using the analyte sensor control unit, regarding a level of an analyte from signals generated by the analyte sensor, and transmitting the collected data from the analyte sensor control unit to a receiver unit. The analyte sensor includes a working electrode, a counter electrode, and a sensing layer disposed on the working electrode, where the sensing layer includes an analyte-responsive enzyme, a redox mediator and a thickener including one or more of urea, a urea derivative, urethane, a urethane derivative, a polyvinyl pyrrolidone polymer and a polyvinyl pyrrolidone polymer derivative. In certain embodiments, the sensing layer has an arcuate profile as measured using a profilometer.

In certain instances, the analyte sensor has a sensitivity that is 90% or more of its initial sensitivity after 14 days or more.

In some cases, the analyte sensor has an average response time that is 35% or more lower than the average response time of a sensor that does not include a thickener.

In certain embodiments, the analyte is glucose.

In some instances, the collecting data includes generating signals from the analyte sensor and processing the signals into data.

In certain embodiments, the data includes the signals from the analyte sensor.

Some embodiments of the method further include activating an alarm if the data indicate an alarm condition.

In some instances, the method further includes administering a drug in response to the data. In certain cases, the drug is insulin.

In certain embodiments, the method further includes obtaining a calibration value from a calibration device to calibrate the data. In some cases, the calibration device is coupled to a display unit. In some instances, the method further includes transmitting the calibration value from a transmitter in the display unit to a receiver in the analyte sensor control unit.

Aspects of the present disclosure also include a method of fabricating an electrode for use in an analyte sensor. The method includes contacting an electrode with a sensing layer, where the sensing layer includes an analyte-responsive enzyme, a redox mediator and a thickener including one or more of urea, a urea derivative, urethane, a urethane derivative, a polyvinyl pyrrolidone polymer and a polyvinyl pyrrolidone polymer derivative.

In some cases, the sensing layer has an arcuate profile as measured using a profilometer.

In certain instances, the analyte sensor has a sensitivity that is 90% or more of its initial sensitivity after 14 days or more.

In some instances, the analyte sensor has an average response time that is 35% or more lower than the average response time of a sensor that does not include a thickener.

In certain cases, the analyte-responsive enzyme and the redox mediator are distributed throughout the sensing layer.

In some embodiments, the method further includes contacting the sensing layer with a membrane that limits flux of the analyte to the sensing layer, where the membrane is disposed over the sensing layer. In certain instances, only the sensing layer comprises the thickener.

In certain embodiments, the analyte sensor is a glucose sensor. In some of these embodiments, the analyte-responsive enzyme comprises a glucose-responsive enzyme. In some cases, the glucose-responsive enzyme includes glucose oxidase. In some instances, the redox mediator comprises a ruthenium-containing complex or an osmium-containing complex.

In certain embodiments, the analyte sensor is an in vivo sensor. In other cases, the analyte sensor is an in vitro sensor.

Aspects of the present disclosure also include an analyte sensor having a working electrode, a counter electrode, and a sensing layer disposed on the working electrode, where the sensing layer includes a glucose-responsive enzyme, a redox mediator and a peptide-based enzyme stabilizer.

In certain embodiments, at least a portion of the analyte sensor is adapted to be subcutaneously positioned in a subject.

In some cases, the analyte sensor has a sensitivity that is 90% or more of its initial sensitivity after 7 days or more.

In some instances, the analyte sensor has an average response time that is 20% or more lower than the average response time of a sensor that does not include an enzyme stabilizer.

In certain cases, the glucose-responsive enzyme comprises glucose oxidase. In some of these cases, the glucose oxidase is in a reduced form. In other embodiments, the glucose oxidase is in an oxidized form.

In certain instances, the peptide-based enzyme stabilizer includes glutathione or a glutathione derivative.

In some cases, the peptide-based enzyme stabilizer includes glutathione.

In some instances, the redox mediator includes a ruthenium-containing complex or an osmium-containing complex.

In certain embodiments, the analyte sensor further includes a membrane disposed over the sensing layer.

In some cases, the analyte sensor is an in vivo sensor. In other embodiments, the analyte sensor is an in vitro sensor.

Aspects of the present disclosure also include a method for monitoring a level of an analyte in a subject. The method includes positioning at least a portion of an analyte sensor into skin of a subject, and determining a level of an analyte over a period of time from signals generated by the analyte sensor, where the determining over a period of time provides for monitoring the level of the analyte in the subject. The analyte sensor includes a working electrode, a counter electrode, and a sensing layer disposed on the working electrode, where the sensing layer includes a glucose-responsive enzyme, a redox mediator and peptide-based enzyme stabilizer.

In certain embodiments, the analyte sensor has a sensitivity that is 90% or more of its initial sensitivity after 7 days or more.

In some instances, the analyte sensor has an average response time that is 20% or more lower than the average response time of a sensor that does not include an enzyme stabilizer.

In some cases, the glucose-responsive enzyme comprises glucose oxidase. In certain instances, the glucose oxidase is in a reduced form. In other instances, the glucose oxidase is in an oxidized form.

In certain embodiments, the peptide-based enzyme stabilizer includes glutathione or a glutathione derivative.

In some cases, the peptide-based enzyme stabilizer includes glutathione.

In certain instances, the redox mediator includes a ruthenium-containing complex or an osmium-containing complex.

In some embodiments, the analyte sensor further includes a membrane disposed over the sensing layer.

Aspects of the present disclosure also include a method for monitoring a level of an analyte using an analyte monitoring system. The method includes inserting at least a portion of an analyte sensor into skin of a patient, attaching an analyte sensor control unit to the skin of the patient, coupling a plurality of conductive contacts of the analyte sensor control unit to a plurality of contact pads of the analyte sensor, collecting data, using the analyte sensor control unit, regarding a level of an analyte from signals generated by the analyte sensor, and transmitting the collected data from the analyte sensor control unit to a receiver unit. The analyte sensor includes a working electrode, a counter electrode, and a sensing layer disposed on the working electrode, where the sensing layer includes a glucose-responsive enzyme, a redox mediator and peptide-based enzyme stabilizer.

In certain embodiments, the analyte sensor has a sensitivity that is 90% or more of its initial sensitivity after 7 days or more.

In some cases, the analyte sensor has an average response time that is 20% or more lower than the average response time of a sensor that does not include an enzyme stabilizer.

In certain embodiments, the glucose-responsive enzyme includes glucose oxidase. In some of these embodiments, the glucose oxidase is in a reduced form. In other embodiments, the glucose oxidase is in an oxidized form.

In certain instances, the peptide-based enzyme stabilizer includes glutathione or a glutathione derivative.

In some cases, the peptide-based enzyme stabilizer includes glutathione.

In some instances, the analyte is glucose.

In certain embodiments, the collecting data includes generating signals from the analyte sensor and processing the signals into data.

In some instances, the data includes the signals from the analyte sensor.

In certain instances, the method further includes activating an alarm if the data indicates an alarm condition.

In some cases, the method further includes administering a drug in response to the data.

In some embodiments, the drug is insulin.

In certain cases, the method further includes obtaining a calibration value from a calibration device to calibrate the data. The some of these cases, the calibration device is coupled to a display unit. In some instances, the method further includes transmitting the calibration value from a transmitter in the display unit to a receiver in the analyte sensor control unit.

Aspects of the present disclosure further include a method of fabricating an electrode for use in an analyte sensor. The method includes contacting an electrode with a sensing layer, where the sensing layer includes a glucose-responsive enzyme, a redox mediator and peptide-based enzyme stabilizer.

In some cases, the analyte sensor has a sensitivity that is 90% or more of its initial sensitivity after 7 days or more.

In certain embodiments, the analyte sensor has an average response time that is 20% or more lower than the average response time of a sensor that does not include an enzyme stabilizer.

In certain cases, the glucose-responsive enzyme comprises glucose oxidase. In some of these cases, the glucose oxidase is in a reduced form. In other embodiments, the glucose oxidase is in an oxidized form.

In certain instances, the peptide-based enzyme stabilizer includes glutathione or a glutathione derivative.

In some embodiments, the peptide-based enzyme stabilizer includes glutathione.

In some instances, the redox mediator includes a ruthenium-containing complex or an osmium-containing complex.

In certain embodiments, the method further includes contacting the sensing layer with a membrane, wherein the membrane is disposed over the sensing layer.

In some cases, the analyte sensor is an in vivo sensor. In other embodiments, the analyte sensor is an in vitro sensor.

Embodiments of the present disclosure also include transition metal complexes having at least one pyridine boronic acid ligand. Transition metal complexes chemically bonded to a polymer are described. In addition, methods for preparing the transition metal complexes and employing the transition metal complexes as electron mediators in an electrochemical sensor are also disclosed.

Aspects of the present disclosure include a transition metal complex having the formula:

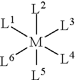

where M is cobalt, iron, osmium, ruthenium, nickel or vanadium;

L₁ is a pyridine boronic acid;

L₂ is a negatively charged ligand; and

L₃, L₄, L₅ and L₆ are combined to form two bidentate ligands which are independently:

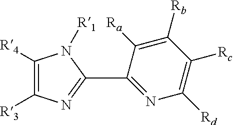

where R′₁ is a substituted or an unsubstituted alkyl, alkenyl, or aryl;

Rₐ and R_{b} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl;

R′₃, R′₄ are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R′₃ and R′₄ forms a saturated or unsaturated 5- or 6-membered ring; and

R_{c}, and R_{d} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R_{c} and R_{d} forms a saturated or unsaturated 5- or 6-membered ring.

In certain embodiments, L₁ is:

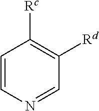

where R^{c} is independently —H, —B(OH)₂ or a substituted or unsubstituted borinate ester; and

where R^{d} is independently —H, —B(OH)₂ or a substituted or unsubstituted borinate ester.

In certain instances, R′₁ is a substituted or an unsubstituted C1-C12 alkyl or alkenyl.

In some cases, R′₁ is methyl.

In some instances, R′₁ is a substituted or an unsubstituted aryl.

In certain cases, R′₁ is a substituted or an unsubstituted phenyl.

In some embodiments, R′₁ is a phenyl substituted with a substituent selected from a group consisting of —Cl, —F, —CN, amino, carboxy, C1-C6 alkyl, C1-C6 alkylthio, C1-C6 alkylamino, C1-C6 dialkylamino, C1-C6 alkylaminocarbonyl, C1-C6 alkoxy, C1-C6 alkoxycarbonyl, and C1-C6 alkylcarboxamido.

In certain embodiments, R′₃, R′₄, Rₐ, R_{b}, R_{c}, and R_{d} are independently —H or substituted or unsubstituted alkyl.

In some instances, the transition metal complex has the formula:

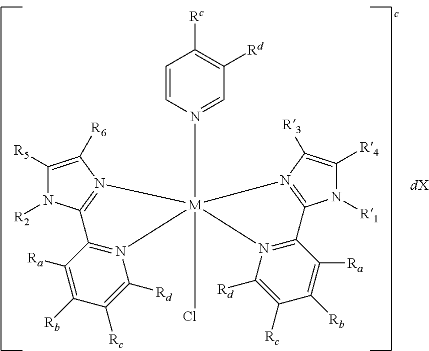

where c is a negative, neutral, or positive charge represented by −1 to −5, 0, or +1 to +5, inclusive, respectively;

d is a number of counter ions, X, from 1 to 5, inclusive

M is cobalt, iron, osmium, ruthenium, nickel or vanadium;

R′₁ is a substituted or an unsubstituted alkyl, alkenyl, or aryl;

Rₐ and R_{b} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl;

R′₃, R′₄ are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R′₃ and R′₄ forms a saturated or unsaturated 5- or 6-membered ring;

R_{c}, and R_{d} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R_{c} and R_{d} forms a saturated or unsaturated 5- or 6-membered ring;

R^{c} is independently —H, —B(OH)₂ or a substituted or unsubstituted borinate ester; and

R^{d} is independently —H, —B(OH)₂ or a substituted or unsubstituted borinate ester.

In certain embodiments, c is 0, +1 or +2.

In some cases, d is 2.

In some instances, X is an anion selected from a group consisting of halides, sulfates, phosphates, hexafluorophosphates and tetrafluoroborates.

In some embodiments, X is chloride.

In certain instances, M is osmium, R′₁ is a substituted or an unsubstituted C1-C6 alkyl, R′₃ and R′₄ are independently —H, Rₐ, R_{b}, R_{c} and R_{d} are independently —H or Cl alkyl, and c is a negative, neutral, or positive charge represented by −1 to −5, 0, or +1 to +5, inclusive, respectively.

In certain cases, L₂ is selected from a group consisting of —CN, —SCN, —OH, halide, alkoxy, alkylthio, and phenoxide.

In some instances, the complex has a redox potential of from about 0 mV to about −200 mV, inclusive, relative to a Ag/AgCl reference electrode.

In certain cases, at least one of L₁, L₂, L₃, L₄, L₅ and L₆ are coupled to a polymeric backbone.

In some embodiments, the polymeric backbone includes L₁ of the complex.

Aspects of the present disclosure further include a sensor that includes a working electrode, a counter electrode, and a mediator complex disposed on the working electrode, the mediator complex having the formula:

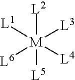

wherein M is cobalt, iron, osmium, ruthenium, nickel or vanadium;

L₁ is a pyridine boronic acid;

L₂ is a negatively charged ligand; and

L₃, L₄, L₅ and L₆ are combined to form two bidentate ligands which are independently:

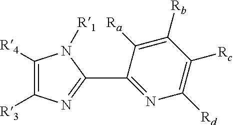

where R′₁ is a substituted or an unsubstituted alkyl, alkenyl, or aryl;

Rₐ and R_{b} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl;

R′₃, R′₄ are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R′₃ and R′₄ forms a saturated or unsaturated 5- or 6-membered ring; and

R_{c}, and R_{d} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R_{c} and R_{d} forms a saturated or unsaturated 5- or 6-membered ring.

In certain embodiments, L₁ is:

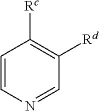

where R^{c} is independently —H, —B(OH)₂ or a substituted or unsubstituted borinate ester; and

where R^{d} is independently —H, —B(OH)₂ or a substituted or unsubstituted borinate ester.

In some cases, the sensor is capable of generating an analyte responsive signal in response to electrolysis of an analyte in the absence of an analyte responsive enzyme.

In some instances, R′₁ is a substituted or an unsubstituted C1-C12 alkyl or alkenyl.

In certain cases, R′₁ is methyl.

In some embodiments, R′₁ is a substituted or an unsubstituted aryl.

In certain instances, R′₁ is a substituted or an unsubstituted phenyl.

In some cases, R′₁ is a phenyl substituted with a substituent selected from a group consisting of —Cl, —F, —CN, amino, carboxy, C1-C6 alkyl, C1-C6 alkylthio, C1-C6 alkylamino, C1-C6 dialkylamino, C1-C6 alkylaminocarbonyl, C1-C6 alkoxy, C1-C6 alkoxycarbonyl, and C1-C6 alkylcarboxamido.

In some instances, R′₃, R′₄, Rₐ, R_{b}, R_{c}, and R_{d} are independently —H or substituted or unsubstituted alkyl.

In certain embodiments, the transition metal complex has the formula:

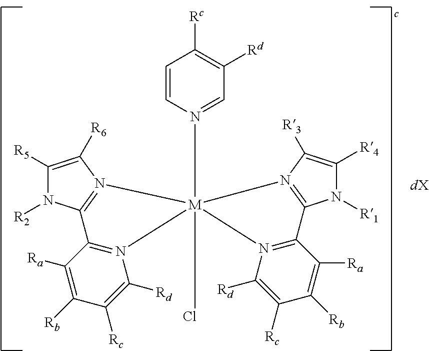

where c is a negative, neutral, or positive charge represented by −1 to −5, 0, or +1 to +5, inclusive, respectively;

d is a number of counter ions, X, from 1 to 5, inclusive

M is cobalt, iron, osmium, ruthenium, nickel or vanadium;

R′₁ is a substituted or an unsubstituted alkyl, alkenyl, or aryl;

Rₐ and R_{b} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl;

R′₃, R′₄ are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R′₃ and R′₄ forms a saturated or unsaturated 5- or 6-membered ring;

R_{c}, and R_{d} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R_{c} and R_{d} forms a saturated or unsaturated 5- or 6-membered ring;

R^{c} is independently —H, —B(OH)₂ or a substituted or unsubstituted borinate ester; and

R^{d} is independently —H, —B(OH)₂ or a substituted or unsubstituted borinate ester.

In certain cases, c is 0, +1 or +2.

In some instances, d is 2.

In certain embodiments, X is an anion selected from a group consisting of halides, sulfates, phosphates, hexafluorophosphates and tetrafluoroborates.

In some cases, X is chloride.

In certain instances, M is osmium, R′₁ is a substituted or an unsubstituted C1-C6 alkyl, R′₃ and R′₄ are independently —H, Rₐ, R_{b}, R_{c} and R_{d} are independently —H or Cl alkyl, and c is a negative, neutral, or positive charge represented by −1 to −5, 0, or +1 to +5, inclusive, respectively.

In certain cases, L₂ is selected from a group consisting of —CN, —SCN, —OH, halide, alkoxy, alkylthio, and phenoxide.

In some cases, the sensor has a redox potential of from about 0 mV to about −200 mV, inclusive, relative to a Ag/AgCl reference electrode.

In some instances, at least one of L₁, L₂, L₃, L₄, L₅ and L₆ is coupled to a polymeric backbone.

In certain instances, the polymeric backbone includes L₁ of the complex.

In some embodiments, the working electrode further includes an analyte responsive enzyme. In some of these embodiments, the analyte-responsive enzyme is glucose dehydrogenase (GDH).

In some instances, the mediator is disposed in the measurement zone.

In some cases, the measurement zone and the sample chamber are sized to contain a volume of no more than about 1 μL of the sample.

In certain cases, the working electrode and counter electrode are separated by a distance of 25 to 1000 μm.

Aspects of the present disclosure further include a method for determining a concentration of glucose in a sample. The method includes the steps of contacting a sample with a sensor, generating a sensor signal at the working electrode, and determining the concentration of the glucose using the sensor signal. The sensor includes a working electrode and a counter electrode, where the working electrode includes a mediator complex. The mediator complex has

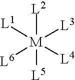

where M is cobalt, iron, osmium, ruthenium, nickel or vanadium;
L₁ is:

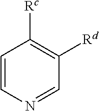

where R^{c} is independently —H, —B(OH)₂ or a substituted or unsubstituted borinate ester; and
where R^{d} is independently —H, —B(OH)₂ or a substituted or unsubstituted borinate ester; and
L₂ is a negatively charged ligand; and
L₃, L₄, L₅ and L₆ are combined to form two bidentate ligands which are independently:

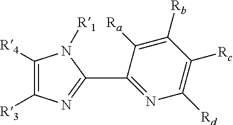

where R′₁ is a substituted or an unsubstituted alkyl, alkenyl, or aryl;
   Rₐ and R_{b} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl;
   R′₃, R′₄ are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R′₃ and R′₄ forms a saturated or unsaturated 5- or 6-membered ring; and
   R_{c}, and R_{d} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R_{c} and R_{d} forms a saturated or unsaturated 5- or 6-membered ring.

In certain embodiments, the background signal that is generated by the mediator is no more than the signal generated by oxidation or reduction of the average normal physiological amount of analyte.

In some cases, determining the concentration of the analyte includes determining the concentration of the analyte by coulometry, amperometry, potentiometry, chronoamperometry, chronopotentiometry, a Cotrell measurement technique or cyclic voltametry using the sensor signal.

In certain instances, determining the concentration of the analyte includes determining the concentration of the analyte by cyclic voltametry using the sensor signal.

In some cases, the method further includes providing calibration data on a batch of the electrochemical sensors to a measurement instrument, the calibration data including information related to a magnitude of a background charge for the batch of the electrochemical sensors, where the step of determining the concentration of the analyte includes determining the concentration of the analyte using the sensor signal and the calibration data.

In some embodiments, the method further includes observing a signal from an indicator electrode to signify that the measurement zone contains sample.

In some cases, generating a sensor signal at the working electrode includes applying a potential between the working electrode and the counter electrode to electrolyze the analyte in the sample, and generating an analyte-responsive signal from the sensor in response to electrolysis of the analyte in the sample.

In certain instances, the sensor generates the analyte responsive signal in response to electrolysis of the analyte in the absence of an enzyme.

In some cases, the working electrode further includes an analyte responsive enzyme.

In some instances, the analyte-responsive enzyme is glucose dehydrogenase (GDH).

Aspects of the present disclosure also include a method of manufacturing a sensor. The method includes forming a plurality of working electrodes on a first substrate, applying a mediator complex to the working electrode, forming a plurality of counter electrodes on a second substrate, disposing a spacer layer on one of the first and second substrates, laminating the first and second substrates together, and separating a plurality of electrochemical sensors from the laminated substrates. The mediator complex has the formula:

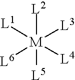

wherein M is cobalt, iron, osmium, ruthenium, nickel or vanadium;
L₁ is:

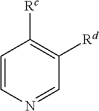

where R^{c} is independently —H, —B(OH)₂ or a substituted or unsubstituted borinate ester; and
where R^{d} is independently —H, —B(OH)₂ or a substituted or unsubstituted borinate ester; and
L₂ is a negatively charged ligand; and
L₃, L₄, L₅ and L₆ are combined to form two bidentate ligands which are independently:

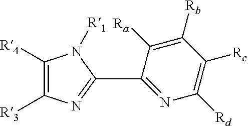

where R′₁ is a substituted or an unsubstituted alkyl, alkenyl, or aryl;
Rₐ and R_{b} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl;
R′₃, R′₄ are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R′₃ and R′₄ forms a saturated or unsaturated 5- or 6-membered ring; and
R_{c}, and R_{d} are independently —H, —F, —Cl, —Br, —I, —NO₂, —CN, —CO₂H, —SO₃H, —NHNH₂, —SH, —OH, —NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl, or a combination of R_{c} and R_{d} forms a saturated or unsaturated 5- or 6-membered ring.

In certain embodiments, a portion of the spacer layer is removed to define a sample chamber region after the spacer layer is disposed on one of the first and second substrates, wherein the sample chamber comprises a measurement zone positioned adjacent to the working electrode and the counter electrode.

In certain cases, the mediator is disposed in the measurement zone. In some of these cases, the measurement zone and the sample chamber are sized to contain a volume of no more than about 1 μL of the sample.

In certain instances, the sensor is configured so that a background signal generated by the mediator is no more than the signal generated by oxidation or reduction of the average normal physiological amount of analyte.

In certain cases, the working electrode and counter electrode are separated by a distance of 25 to 1000 μm.

In some embodiments, the first substrate is a first region of a substrate and the second substrate is a second region of the substrate, and the method further includes folding the substrate to overlay the first and second regions of the substrate.

In certain embodiments, separating the plurality of electrochemical sensors includes cutting the first and second substrates to separate the electrochemical sensors and to define at least one end of the sample chamber of the electrochemical sensors.

In some instances, the method further includes forming a plurality of indicator electrodes on one of the first and second substrates.

In some cases, the working electrode further includes an analyte responsive enzyme.

In certain instances, the analyte-responsive enzyme is glucose dehydrogenase (GDH).

### BRIEF DESCRIPTION OF THE DRAWINGS

A detailed description of various embodiments of the present disclosure is provided herein with reference to the accompanying drawings, which are briefly described below. The drawings are illustrative and are not necessarily drawn to scale. The drawings illustrate various embodiments of the present disclosure and may illustrate one or more embodiment(s) or example(s) of the present disclosure in whole or in part. A reference numeral, letter, and/or symbol that is used in one drawing to refer to a particular element may be used in another drawing to refer to a like element.

FIG. 1 shows a block diagram of an embodiment of an analyte monitoring system according to embodiments of the present disclosure.

FIG. 2 shows a block diagram of an embodiment of a data processing unit of the analyte monitoring system shown in FIG. 1.

FIG. 3 shows a block diagram of an embodiment of the primary receiver unit of the analyte monitoring system of FIG. 1.

FIG. 4 shows a schematic diagram of an embodiment of an analyte sensor according to the embodiments of the present disclosure.

FIGS. 5A-5B show a perspective view and a cross sectional view, respectively, of an embodiment an analyte sensor.

FIG. 6 shows a profilometer graph of a spot of an embodiment of a sensing layer formulation on a gold substrate, there the sensing layer formulation does not include a thickener according to an embodiment of the present disclosure.

FIG. 7 shows a profilometer graph of a spot of an embodiment of a sensing layer formulation on a gold substrate, there the sensing layer formulation includes 3% (v/v) of a thickener (BYK-420), according to an embodiment of the present disclosure. The profilometer graph illustrates the homogeneity and uniformity of solution distribution that results when the sensing layer includes a thickener according to embodiments of the present disclosure.

FIG. 8 shows graphs of sensitivity/slope (nA/mM) for sensing layer formulations that included 1.5% BYK-420 at 0 days and at 14 days, according to embodiments of the present disclosure.

FIG. 9 shows graphs of average response time (sec) for sensing layer formulations that included 1.5% BYK-420 at 0 days and at 14 days, according to embodiments of the present disclosure.

FIG. 10A shows graphs of sensitivity/slope (nA/mM) for sensing layer formulations that included 3% BYK-420 at 0 days, according to embodiments of the present disclosure. FIG. 10B shows a t-test graph for the data from experiments shown in FIG. 10A.

FIG. 11A shows graphs of average response time (sec) for sensing layer formulations that included 3% BYK-420 at 0 days, according to embodiments of the present disclosure. FIG. 11B shows a t-test graph for the data from experiments shown in FIG. 11A.

FIG. 12 shows graphs of sensitivity/slope (nA/mM) for sensing layer formulations that included 0.25% and 0.5% glutathione at 0 days and at 7 days, according to embodiments of the present disclosure.

FIG. 13 shows graphs of average response time (sec) for sensing layer formulations that included 0.25% and 0.5% glutathione at 0 days and at 7 days, according to embodiments of the present disclosure.

FIG. 14 shows graphs of sensitivity (nA) vs. time for sensing layer formulations that included glutathione, according to embodiments of the present disclosure.

### DETAILED DESCRIPTION

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting, since the scope of the embodiments of the invention will be defined by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives, is merely illustrative, not limiting, unless implicitly or explicitly understood or stated otherwise.

Various terms are described below to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular analytes, bodily or tissue fluids, blood or capillary blood, or sensor constructs or usages, unless implicitly or explicitly understood or stated otherwise, as such may vary.

The publications discussed herein are provided solely for their disclosure prior to the filing date of the application. Nothing herein is to be construed as an admission that the embodiments of the invention are not entitled to antedate such publication by virtue of prior invention. Further, the dates of publication provided may be different from the actual publication dates which may need to be independently confirmed.

### Systems and Methods Using Thickeners

Embodiments of the present disclosure relate to methods and devices for improving the uniformity of distribution of a sensing layer of a sensor by inclusion of a thickener, where the sensing layer is disposed on a working electrode of the sensor, such as in vivo and/or in vitro analyte sensors, including, for example, continuous and/or automatic in vivo analyte sensors. Embodiments of the present disclosure provide for inclusion of a thickener in a solution, such as a sensing layer formulation, resulting in an increase in viscosity of the solution, thereby reducing uneven evaporation of the sensing layer and/or reducing loss of sensitivity over time. Also provided are systems and methods of using the analyte sensors in analyte monitoring.

Embodiments of the present disclosure are based on the discovery that the addition of a thickener to solution formulations used in the manufacture of in vivo and/or in vitro biosensors improves uniformity and/or distribution of the sensing layer of the sensor (e.g., an enzyme-containing sensing layer). Biocompatible sensing layers of embodiments of the present disclosure can include thickeners, e.g., compounds or compositions that increase the viscosity of the sensing layer formulation. In some cases, the thickener increases the viscosity of the sensing layer formulation by forming a gel. In certain instances, the gel is a hydrogel.

During the manufacturing process for the subject analyte sensors, an aqueous solution (e.g., a sensing layer) is contacted with a surface of a substrate (e.g., a surface of a working electrode), forming a deposition of the solution on the surface of the substrate. In some cases, the solution is allowed to dry and cure. Without being limited to any particular theory, in certain instances, during the drying, the constituents of the solution may tend to migrate towards the outer edges of the deposition due to a faster rate of evaporation at the thinner peripheral edges of the deposition. This results in a greater concentration of the constituents of the solution at the peripheral edges of the deposition, resulting in a so-called “coffee ring” effect.

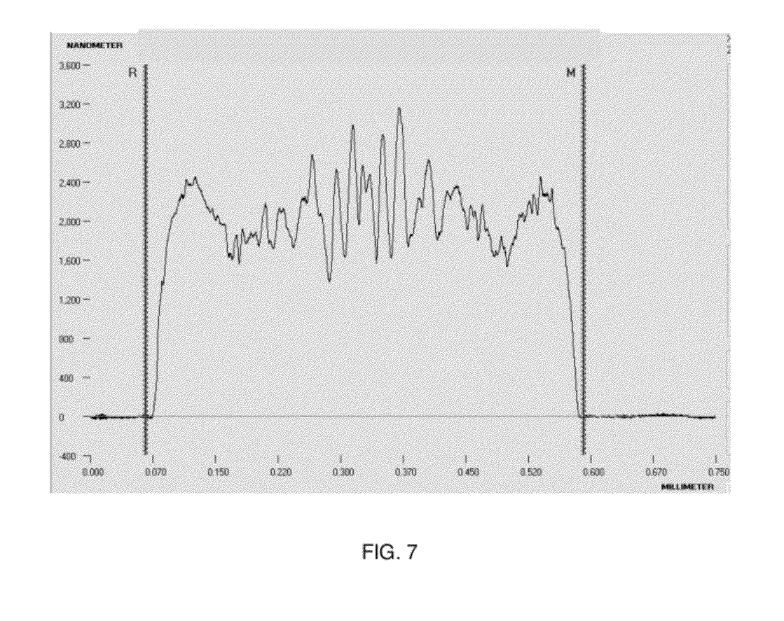

In certain embodiments of the present disclosure, the thickeners increase the viscosity of the solution. In certain instances, the thickener increases the viscosity of the solution by forming a gel, such as, but not limited to, a hydrogel. Inclusion of a thickener in the sensing layer formulation may result in a reduction, and in some cases, complete elimination of the “coffee ring” effect (see e.g., the homogenous and uniform distribution of each sample in FIG. 7). In some cases, this results in a more uniform distribution of the constituents of the solution deposited on the substrate upon drying and curing as compared to a solution lacking the thickener. In some embodiments, this results in an increase in the sensitivity of the sensor and/or a reduction in loss of sensitivity over time as compared to a sensing layer formulation without the thickener. In some instances, inclusion of a thickener in the sensing layer formulation also results in a smoother surface of the solution upon drying and curing as compared to a solution lacking the thickener. This, in turn, improves the coefficient of variation and the overall manufacturing process of the sensor and overall system.

In certain embodiments, the thickener increases the sensitivity of the sensing layer formulation as compared to sensors that do not include a thickener. In certain instances, sensors that include a sensing layer formulation with a thickener have a sensitivity of 0.7 nA/mM or more, or 0.8 nA/mM or more, such as 0.9 nA/mM or more, including 1 nA/mM or more, for instance 1.1 nA/mM or more, or 1.2 nA/mM or more, or 1.3 nA/mM or more. In some cases, sensors that include a sensing layer formulation with a thickener have a sensitivity ranging from 0.7 nA/mM to 1.3 nA/mM, such as from 0.7 nA/mM to 1.2 nA/mM, including from 0.8 nA/mM to 1.1 nA/mM, or from 0.9 nA/mM to 1.1 nA/mM.

In some cases, a sensor that includes a sensing layer formulation that includes a thickener as disclosed herein has an initial sensitivity. The sensor may have a sensitivity that is 90% or more of the initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. For example, the sensor may maintain 95% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In some cases, the sensor maintains 97% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In certain instances, the sensor may maintain 99% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more.

Sensors that include a sensing layer formulation with a thickener may also have an average response time that is lower than sensors that do not include a thickener as described herein. In certain embodiments, sensors that include a sensing layer formulation with a thickener have an average response time that is 5% to 60% lower, such as 10% to 55% lower, including 15% to 50% lower, or 20% to 45% lower than the average response time of a sensor that does not include a thickener as described herein. For example, sensors that include a sensing layer formulation with a thickener may have an average response time that is 5% or more, such as 10% or more, 25% or more, 35% or more, or 50% or more lower than the average response time of a sensor that does not include a thickener as described herein. In some cases, sensors that include a sensing layer formulation with a thickener have an average response time of 250 sec or less, such as 225 sec or less, including 200 sec or less, for instance 175 sec or less, or 150 sec or less, or 125 sec or less.

In certain embodiments, when a solution including the thickener of the present disclosure is deposited on the surface of a substrate, the resulting deposition has a substantially arcuate profile as measured using a profilometer (see e.g., FIG. 7). By arcuate is meant that a cross-sectional profile of the deposition has a curved or rounded shape, e.g., an arc shape. In comparison, depositions exhibiting the “coffee ring” effect have cross-sectional profiles that may include more than one local maxima and/or local minima (i.e., peaks and valleys). For example, depositions having a “coffee ring” effect may have profiles that include local maxima near the peripheral edges of the deposition and a local minimum therebetween. In certain embodiments, solutions including a thickener have a reduction in the “coffee ring” effect as compared to a control solution that does not include a thickener.

Examples of thickeners suitable for use with the subject methods, compositions and kits include, but are not limited to, thickeners that include urea or a urea derivative. Other examples of thickeners suitable for use with the subject methods, compositions and kits include, but are not limited to, thickeners that include urethane or a urethane derivative. Additional examples of thickeners suitable for use with the subject methods, compositions and kits include, but are not limited to, thickeners that include , a polyvinyl pyrrolidone polymer or a polyvinyl pyrrolidone polymer derivative. Embodiments of the sensing layer formulations may also include combinations of thickeners, such as, but not limited to, combinations including one or more of urea, a urea derivative, urethane, a urethane derivative, a polyvinyl pyrrolidone polymer and a polyvinyl pyrrolidone polymer derivative. In certain embodiments, the thickener is a modified urea-based polymer thickener, such as, for example, BYK-420 (BYK Chemie GmbH, Wesel, Germany).

The thickener may be included in any component of a sensor that can benefit from improvement of the uniformity of distribution of the constituents of a solution deposited on a surface of a substrate. Embodiments include, but are not limited to, formulations that provide reagents such as an enzyme or the like, such as a sensing layer having an analyte-responsive enzyme. Such components may be sensitive to the formation of crinkles and creases upon curing, giving an “orange peel” effect, such that the surface of the layer may resemble an orange peel. In addition, the component formulation of a sensor when contacted to the sensor (e.g., by dip coating, spray coating, drop deposition, and the like) and cured may form a brittle shell. This phenomenon may give the component layer a brittleness that may cause it to crack, break down and/or peel off of the substrate. These characteristics may cause the sensing layer to slough, chip and peel off carbon substrates and other substrates. In some instances, this chipping can result in the undesirable deposition of residual pieces of the sensing layer in vivo. In addition, as described above, the components of a solution are also sensitive to migrating and settling along the outer perimeter of the deposition, referred to as formation of a “coffee ring” effect on the substrate.

Additional embodiments of a sensor that may be suitably formulated with a thickener are described in U.S. Pat. Nos. 5,262,035, 5,262,305, 6,134,461, 6,143,164, 6,175,752, 6,338,790, 6,579,690, 6,605,200, 6,605,201, 6,654,625, 6,736,957, 6,746,582, 6,932,894, 7,090,756 as well as those described in U.S. patent application Ser. Nos. 11/701,138, 11/948,915, 12/625,185, 12/625,208, and 12/624,767, the disclosures of all of which are incorporated herein by reference in their entirety. Moreover, the present invention may be incorporated into battery-powered or self-powered analyte sensors, in one embodiment the analyte sensor is a self-powered sensor, such as disclosed in U.S. patent application Ser. No. 12/393,921 (Publication No. 2010/0213057).

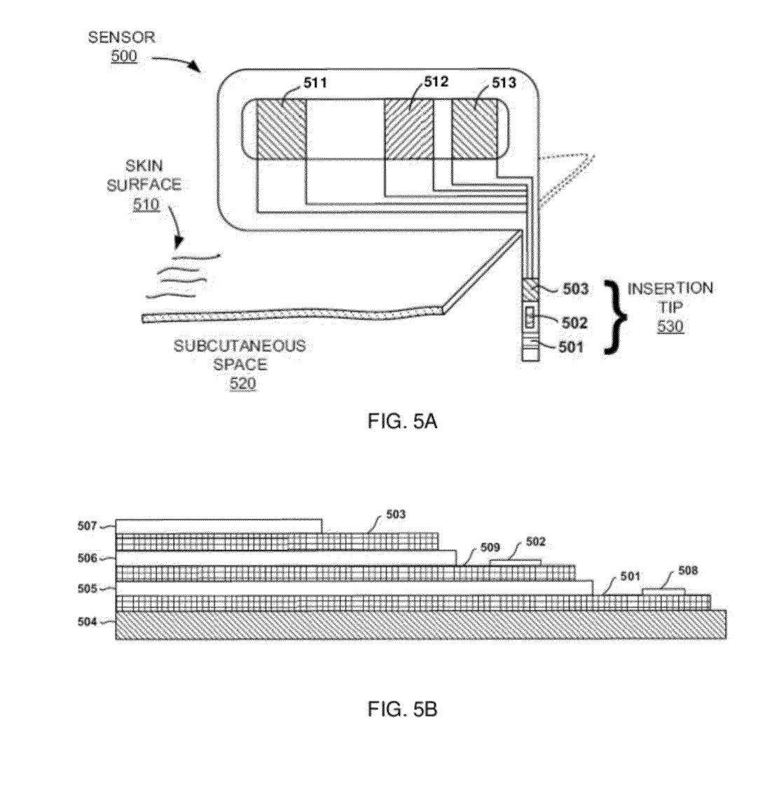

In some embodiments, the thickener is formulated with a sensing layer that is disposed on a working electrode. An embodiment of a sensing layer may be described as the area shown schematically in FIG. 5B as 508. The sensing layer may be described as the active chemical area of the biosensor. The sensing layer formulation, which can include a glucose-transducing agent, may include, for example, among other constituents, a redox mediator, such as, for example, a hydrogen peroxide or a transition metal complex, such as a ruthenium-containing complex or an osmium-containing complex, and an analyte-responsive enzyme, such as, for example, a glucose-responsive enzyme (e.g., glucose oxidase, glucose dehydrogenase, etc.) or lactate-responsive enzyme (e.g., lactate oxidase). In certain embodiments, the sensing layer includes glucose oxidase. The sensing layer may also include other optional components, such as, for example, a polymer and a bi-functional, short-chain, epoxide cross-linker, such as polyethylene glycol (PEG).

In certain instances, the analyte-responsive enzyme is distributed throughout the sensing layer. For example, the analyte-responsive enzyme may be distributed uniformly throughout the sensing layer, such that the concentration of the analyte-responsive enzyme is substantially the same throughout the sensing layer. In some cases, the sensing layer may have a homogeneous distribution of the analyte-responsive enzyme. In certain embodiments, the redox mediator is distributed throughout the sensing layer. For example, the redox mediator may be distributed uniformly throughout the sensing layer, such that the concentration of the redox mediator is substantially the same throughout the sensing layer. In some cases, the sensing layer may have a homogeneous distribution of the redox mediator. In certain embodiments, both the analyte-responsive enzyme and the redox mediator are distributed uniformly throughout the sensing layer, as described above.

Any suitable proportion of thickener may be used with a sensing layer, where the specifics will depend on, e.g., the particular sensing layer formulation, etc. In certain embodiments, the thickener may range from 1.5% to 3% (v/v) of the total biosensor sensing layer formulation. For example, such thickeners may range from 0.1% to 25% (v/v) of the total biosensor sensing layer formulation, such as from 0.5% to 10% (v/v), including from 1% to 5% (v/v), for instance from 1.5% to 3% (v/v), and the like. In certain cases, only the sensing layer includes the thickener. For instance, the thickener may only be included in the sensing layer and substantially excluded from any of the other layers of the sensor, such as, but not limited to, one or more membrane layers disposed over the sensing layer.

### Systems and Methods Using Enzyme Stabilizers

Additional embodiments of the present disclosure relate to methods and devices for improving the sensitivity of a sensing layer of a sensor by inclusion of an enzyme stabilizer, where the sensing layer is disposed on a working electrode of the sensor, such as in vivo and/or in vitro analyte sensors, including, for example, continuous and/or automatic in vivo analyte sensors. Embodiments of the present disclosure provide for inclusion of an enzyme stabilizer in a solution, such as a sensing layer formulation, resulting in an increase in the stability of the enzyme in the solution, thereby increasing the sensitivity of the sensor and/or maintaining the sensitivity of the sensor over time. Also provided are systems and methods of using the analyte sensors in analyte monitoring.

Embodiments of the present disclosure are based on the discovery that the addition of an enzyme stabilizer to solution formulations used in the manufacture of in vivo and/or in vitro biosensors improves the stability of an analyte-responsive enzyme in the sensing layer of the sensor. In some instances, the increase in the stability of the analyte-responsive enzyme leads to a corresponding decrease in degradation of the analyte-responsive enzyme over time. Biocompatible sensing layers of embodiments of the present disclosure can include enzyme stabilizers, e.g., compounds or compositions that decrease the degradation or loss in activity of the analyte-responsive enzyme in the sensing layer formulation.

During the manufacturing process for the subject analyte sensors, an aqueous solution (e.g., a sensing layer) is contacted with a surface of a substrate (e.g., a surface of a working electrode), forming a deposition of the solution on the surface of the substrate. In some cases, the solution is allowed to dry and cure. Without being limited to any particular theory, in certain instances, after deposition of the sensing layer formulation, the analyte-responsive enzyme in the solution may tend to lose stability, degrade or lose activity slowly over time. This may result in a reduction in the sensitivity of the sensor over time.

In certain embodiments of the present disclosure, the enzyme stabilizer increases the stability of the analyte-responsive enzyme in the sensing layer formulations. Inclusion of an enzyme stabilizer in the sensing layer formulation may result in a reduction, and in some cases, complete elimination of the loss of sensitivity of the analyte-responsive enzyme over time. Inclusion of an enzyme stabilizer in the sensing layer formulation may, in some instances, result in a reduction in the amount of degradation and/or loss of activity of the analyte-responsive enzyme over time. In some embodiments, this results in a reduction in loss of sensitivity over time as compared to a sensing layer formulation without the thickener. In some cases, inclusion of an enzyme stabilizer in the sensing layer formulation results in an increase in sensitivity for the sensor as compared to a sensing layer formulation lacking the enzyme stabilizer. This, in turn, improves the coefficient of variation and the overall manufacturing process of the sensor and overall system.

In certain embodiments, the enzyme stabilizer increases the sensitivity of the sensing layer formulation as compared to sensors that do not include enzyme stabilizers. In some cases, sensors that include a sensing layer formulation with an enzyme stabilizer have a sensitivity of 0.5 nA/mM or more, such as 0.6 nA/mM or more, including 0.7 nA/mM or more, for instance 0.8 nA/mM or more, or 0.9 nA/mM or more, or 1 nA/mM or more, or 1.1 nA/mM or more. In certain instance, sensors that include a sensing layer formulation with an enzyme stabilizer have a sensitivity ranging from 0.5 nA/mM to 1.1 nA/mM, such as from 0.5 nA/mM to 1 nA/mM, including from 0.6 nA/mM to 0.9 nA/mM.

In some cases, a sensor that includes a sensing layer formulation with an enzyme stabilizer as disclosed herein has an initial sensitivity. The sensor may have a sensitivity that is 90% or more of the initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. For example, the sensor may have a sensitivity that is 95% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In certain instances, the sensor may have a sensitivity that is 99% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In certain instances, the sensor may have a sensitivity that is 100% or more, such as 102% or more, or 105% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more.

Sensors that include a sensing layer formulation with an enzyme stabilizer may also have an average response time that is lower than sensors that do not include an enzyme stabilizer as described herein. In certain embodiments, sensors that include a sensing layer formulation with an enzyme stabilizer have an average response time that is 5% to 50% lower, such as 10% to 45% lower, including 15% to 40% lower, or 20% to 35% lower than the average response time of a sensor that does not include an enzyme stabilizer as described herein. For example, sensors that include a sensing layer formulation with an enzyme stabilizer may have an average response time that is 5% or more, such as 10% or more, 25% or more, 35% or more, or 50% or more lower than the average response time of a sensor that does not include an enzyme stabilizer as described herein. In certain embodiments, sensors that include a sensing layer formulation with an enzyme stabilizer have an average response time of 250 sec or less, such as 225 sec or less, including 200 sec or less, for instance 175 sec or less, or 150 sec or less.

Examples of enzyme stabilizers suitable for use with the subject methods, compositions and kits include, but are not limited to, enzyme stabilizers that include a peptide or a peptide derivative. Embodiments of the sensing layer formulations may also include combinations of peptides or peptide derivatives. In certain embodiments, the enzyme stabilizer is a peptide-based enzyme stabilizer, such as, for example, glutathione, a derivative of glutathione, a salt of glutathione or combinations thereof.

The enzyme stabilizer may be included in a sensing layer of a sensor that can benefit from improvement in the stability of the analyte-responsive enzyme in a solution deposited on a surface of a substrate. Embodiments include, but are not limited to, formulations that provide reagents such as an enzyme or the like, such as a sensing layer having an analyte-responsive enzyme. Such sensing layers may be sensitive to degradation of the analyte-responsive enzyme and/or loss of activity of the analyte-responsive enzyme over time after the sensing layer is deposited on the surface of the substrate. Degradation of the analyte-responsive enzyme and/or loss of activity of the analyte-responsive enzyme over time may result in a reduction in the sensitivity of the sensor over time.

Additional embodiments of a sensor that may be suitably formulated with an enzyme stabilizer are described in U.S. Pat. Nos. 5,262,035, 5,262,305, 6,134,461, 6,143,164, 6,175,752, 6,338,790, 6,579,690, 6,605,200, 6,605,201, 6,654,625, 6,736,957, 6,746,582, 6,932,894, 7,090,756 as well as those described in U.S. patent application Ser. Nos. 11/701,138, 11/948,915, 12/625,185, 12/625,208, and 12/624,767, the disclosures of all of which are incorporated herein by reference in their entirety. Moreover, the present invention may be incorporated into battery-powered or self-powered analyte sensors, in one embodiment the analyte sensor is a self-powered sensor, such as disclosed in U.S. patent application Ser. No. 12/393,921 (Publication No. 2010/0213057).

In some embodiments, the enzyme stabilizer is formulated with a sensing layer that is disposed on a working electrode. An embodiment of a sensing layer may be described as the area shown schematically in FIG. 5B as 508. The sensing layer may be described as the active chemical area of the biosensor. The sensing layer formulation, which can include a glucose-transducing agent, may include, for example, among other constituents, a redox mediator, such as, for example, a hydrogen peroxide or a transition metal complex, such as a ruthenium-containing complex or an osmium-containing complex, and an analyte responsive enzyme, such as, for example, a glucose responsive enzyme (e.g., glucose oxidase, glucose dehydrogenase, etc.) or lactate responsive enzyme (e.g., lactate oxidase). In certain embodiments, the sensing layer includes glucose oxidase. The glucose oxidase may be, in some cases, in a reduced form, and in other cases in an oxidized form. The sensing layer may also include other optional components, such as, for example, a polymer and a bi-functional, short-chain, epoxide cross-linker, such as polyethylene glycol (PEG).

As described above, in certain instances, the analyte-responsive enzyme is distributed throughout the sensing layer. For example, the analyte-responsive enzyme may be distributed uniformly throughout the sensing layer, such that the concentration of the analyte-responsive enzyme is substantially the same throughout the sensing layer. In some cases, the sensing layer may have a homogeneous distribution of the analyte-responsive enzyme. In certain embodiments, the redox mediator is distributed throughout the sensing layer. For example, the redox mediator may be distributed uniformly throughout the sensing layer, such that the concentration of the redox mediator is substantially the same throughout the sensing layer. In some cases, the sensing layer may have a homogeneous distribution of the redox mediator. In certain embodiments, both the analyte-responsive enzyme and the redox mediator are distributed uniformly throughout the sensing layer, as described above.

Any suitable proportion of enzyme stabilizer may be used with a sensing layer, where the specifics will depend on, e.g., the particular sensing layer formulation, etc. In certain embodiments, the enzyme stabilizer may range from 0.25% to 0.5% (w/v) of the total biosensor sensing layer formulation. For example, such enzyme stabilizers may range from 0.1% to 10% (w/v) of the total biosensor sensing layer formulation, such as from 0.25% to 5% (w/v), including from 0.25% to 3% (w/v), for instance from 0.25% to 1% (w/v), or from 0.25% to 5% (w/v), and the like. In certain cases, only the sensing layer includes the enzyme stabilizer. For instance, the enzyme stabilizer may only be included in the sensing layer and substantially excluded from any of the other layers of the sensor, such as, but not limited to, one or more membrane layers disposed over the sensing layer.

### Systems and Methods Using Osmium Boronates

Transition metal complexes having at least one pyridine boronic acid ligand are described. Aspects of the disclosure also include transition metal complexes chemically bonded to a polymer. In addition, methods for preparing the transition metal complexes and employing the transition metal complexes as electron mediators in an electrochemical sensor are also disclosed.

Aspects of the osmium boronate complexes are summarized below, and described in greater detail in the subsequent sections.

In some embodiments, the transition metal complexes have the formula:

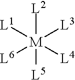

where M is a transition metal, such as for example osmium, nickel, ruthenium, vanadium, cobalt, or iron. In some embodiments L¹, L², L³, L⁴, L⁵, and L⁶ are independently monodentate ligands or may be combined with at least one other ligand to form a multidentate ligand. Ligands may include one or more heterocycles coordinatively, ionically or covalently bonded to M via a heteroatom of the heterocycle. In some embodiments, one or more of ligands L¹, L², L³, L⁴, L⁵, and L⁶ is a pyridine boronic acid ligand, where in some instances, the pyridine boronic acid ligand has the formula:

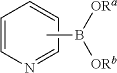

The term “boronic acid” is used in its conventional sense to refer to the broad class of organoborane compounds, such that the boronic acid substituents contain at least one carbon-boron bond. Boronic acid substituents include boronic acid (—B(OH)₂), but may also include alkyl and aryl substituted boronate esters or boronate salts.

In some instances, the pyridine boronic acid ligand may have the formula:

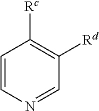

where R^{c} and R^{d} are independently —H or —B(OR¹)₂, where R¹ may be —H, alkyl, alkenyl, or aryl, alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxyamino, or alkylthio.

In some embodiments, transition metal complexes are osmium complexes and include at least one pyridine boronic acid ligand. In some instances, osmium complexes having at least one pyridine boronic acid ligand have the formula:

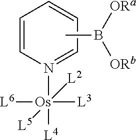

In some embodiments, osmium complexes having at least one pyridine boronic acid ligand have the formula:

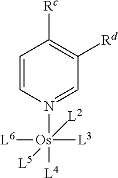

In some embodiments, the transition metal complexes have the formula:

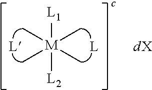

where M is a transition metal, such as for example cobalt, iron, osmium, nickel, ruthenium, or vanadium; c is an integer selected from −1 to −5 or +1 to +5 indicating a negative or positive charge, respectively; X represents at least one counter ion; and d is an integer from 1 to 5 representing the number of counter ions, X.

In some embodiments, L and L′ are bidentate ligands and are selected from the group consisting of:

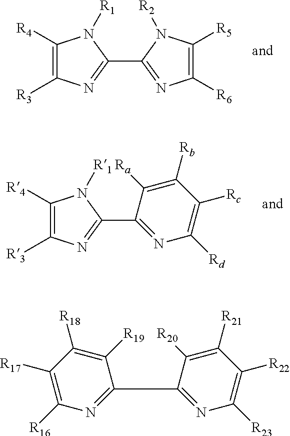

where substituents of the bidentate ligands may be independently —H, —F, —Cl, —Br, —I, —NO₂, —CN, —CO₂H, —SO₃H, —NHNH₂, —SH, —OH, or substituted or unsubstituted alkyl, alkenyl, aryl, alkoxycarbonyl, alkylaminocarbonyl, dialkylaminocarbonyl, alkoxy, —NH₂, alkylamino, dialkylamino, alkanoylamino, arylcarboxamido, hydrazino, alkylhydrazino, hydroxylamino, alkoxyamino, or alkylthio groups.

In some embodiments, transition metal complexes have the formula:

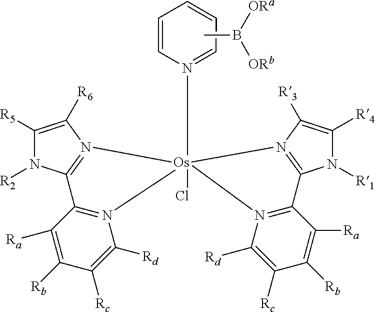

where L₁ is a pyridine boronic acid ligand, L₂ is chloride and L and L′ are 2-(2-pyridyl)-imidazole bidentate ligands where substituents of the bidentate ligands may be independently —H, —F, —Cl, —Br, —I, —NO₂, —CN, —CO₂H, —SO₃H, —NHNH₂, —SH, —OH, or substituted or unsubstituted alkyl, alkenyl, aryl, alkoxycarbonyl, alkylaminocarbonyl, dialkylaminocarbonyl, alkoxy, —NH₂, alkylamino, dialkylamino, alkanoylamino, arylcarboxamido, hydrazino, alkylhydrazino, hydroxylamino, alkoxyamino, or alkylthio groups.

In some embodiments, one or more transition metal mediator complexes are chemically bonded to a polymer. In further embodiments, the transition metal complexes are chemically bonded to a polymer to form a polymeric transition metal complex. Polymeric transition metal complexes are capable of carrying electrons between an analyte and an electrode. As such, these polymers may be useful in electrochemical sensors. In one embodiment, polymeric transition metal complexes include a polymeric backbone and a plurality of transition metal complexes chemically bonded to the polymeric backbone. In other embodiments, polymeric transition metal complexes include a polymeric backbone, a plurality of transition metal complexes and a plurality of spacer moieties. Each of the spacers is covalently coupled to and extending from the polymeric backbone and includes at least one non-cyclic functional group selected from the group consisting of —(CR^{r}R^{s})—, —O—, —S—, —C(O)O—, —S(O)₂NR^{k}—, —OC(O)NR^{m}—, —OC(S)NR^{a}, —C(O)NR^{t}—, —NR^{u}—, —CR^{v}═N—O—, —CR^{q}═NNR^{x}—, and —(SiR^{y}R^{z})—, where R^{r} and R^{s} are independently hydrogen, chlorine, fluorine, or substituted or unsubstituted alkyl, alkoxy, alkenyl, or alkynyl and R^{k}, R^{m}, Rⁿ, R^{t}, R^{u}, R^{v}, R^{w}, R^{x}, R^{y}, and R^{z} are independently hydrogen or substituted or unsubstituted alkyl.

In some embodiments, polymeric transition metal complexes include a plurality of transition metal complexes having at least one pyridine boronic acid ligand, where the transition metal complexes are chemically bonded to a polymer.

Further aspects also include an electrochemical sensor employing transition metal complexes having at least one pyridine boronic acid ligand. In some embodiments, electrochemical sensors may include transition metal complexes having at least one pyridine boronic acid ligand where the transition metal complexes are chemically bonded to a polymer. For example, the polymer chemically bonded to the mediator may be disposed proximate to the working electrode of the electrochemical sensor. In other instances, the polymer chemically bonded to the mediator may be covalently bonded to the working electrode of the electrochemical sensor.

In some embodiments, transition metal complexes and electrochemical sensors of may be employed in sensing applications. The advantageous properties and characteristics of the transition metal complexes described herein make them ideal candidates for use in the electrochemical sensing of analytes in a biological fluid. In some instances, transition metal complexes and electrochemical sensors described herein may be employed to determine the concentration of an analyte (e.g., glucose, ketone bodies, lactate, etc.) in a biological fluid (e.g., blood, subcutaneous tissue fluid, urine, etc.). In some embodiments, electrochemical sensors employing transition metal complexes described herein determine the concentration of the analyte in the biological fluid in the absence of an analyte responsive enzyme.

Aspects of the transition metal complexes are described in greater detail below. In addition, polymeric transition metal complexes which include a plurality of transition metal complexes chemically bonded to a polymer are disclosed. Electrochemical sensors that employ various embodiments of transition metal complexes are also reviewed. Methods for producing and employing the transition metal complexes to determine the concentration of an analyte in a biological fluid by an electrochemical sensor are also described.

Aspects of the present application include transition metal complexes of iron, cobalt, ruthenium, osmium, nickel and vanadium having at least one pyridine ligand substituted with at least one boronic acid. The term “boronic acid” is used in its conventional sense to refer to the broad class of organoborane compounds which contain at least one carbon-boron bond. Boronic acid substituents may include boronic acid (—B(OH)₂), but may also include alkyl, alkenyl, alkynyl and/or aryl substituted boronate esters or boronate salts. In some embodiments, pyridine boronic acid ligands have the formula:

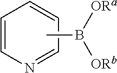

where pyridine boronic acid ligands may contain one or more boronic acid substituents, such as two or more boronic acid substituents, such as three or more boronic acid substituents, such as four of more boronic acid substituents, including five boronic acid substituents.

In certain embodiments, pyridine boronic acid ligands have the formula:

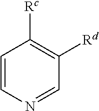

where R^{c} and R^{d} are independently —H or —B(OR¹)₂, where R¹ may be —H, alkyl, alkenyl, alkynyl or aryl, alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxyamino, or alkylthio. In some embodiments, R^{c} is —H and R^{d} is —B(OH)₂. In other embodiments, R^{c} is —B(OH)₂ and R^{d} is —H.

Aspects of the application also include transition metal complexes having the formula:

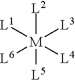

where M is a transition metal, such as for example, osmium, nickel, ruthenium, vanadium, cobalt, or iron, among others. L¹, L², L³, L⁴, L⁵, and L⁶ are ligands and are independently monodentate ligands or two or more of the ligands can be combined to form one or more multidentate ligands.

In some embodiments, one or more of L¹, L², L³, L⁴, L⁵, and L⁶ is a pyridine boronic acid ligand. In certain instances only one of L¹, L², L³, L⁴, L⁵, and L⁶ is a pyridine boronic acid ligand. In other instances, more than one of L¹, L², L³, L⁴, L⁵, and L⁶ are pyridine boronic acid ligands, such as two of L¹, L², L³, L⁴, L⁵, and L⁶ are pyridine boronic acid ligands, such as three of L¹, L², L³, L⁴, L⁵, and L⁶ are pyridine boronic acid ligands, such as four of L¹, L², L³, L⁴, L⁵, and L⁶ are pyridine boronic acid ligands, such as five of L¹, L², L³, L⁴, L⁵, and L⁶ are pyridine boronic acid ligands, including all of L¹, L², L³, L⁴, L⁵, and L⁶ are pyridine boronic acid ligands.

In some embodiments, the transition metal complexes are osmium complexes where L¹ is a pyridine boronic acid ligand. In some instances, osmium complexes having a pyridine boronic acid ligand have the formula:

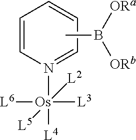

where the pyridine boronic acid ligand may have one or more boronic acid substituents, such as two or more boronic acid substituents, such as three or more boronic acid substituents, such as four of more boronic acid substituents, including five boronic acid substituents.

In some instances, osmium complexes having a pyridine boronic acid ligand may have the formula:

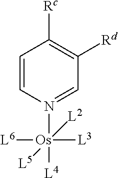

where R^{c} and R^{d} are independently —H or —B(OR¹)₂, where R¹ may be —H, alkyl, alkenyl, alkynyl or aryl, alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxyamino, or alkylthio. In some embodiments, R^{c} is —H and R^{d} is —B(OH)₂. In other embodiments, R^{c} is —B(OH)₂ and R^{d} is —H.

In embodiments where L¹ is a pyridine boronic acid ligand, any combination of monodentate and multidentate ligands can be used for L², L³, L⁴, L⁵, and L⁶. For example, any of L², L³, L⁴, L⁵, and L⁶ can combine to form bidentate ligands such as, for example, bidentate ligands selected from substituted and unsubstituted 2,2′-biimidazoles, 2-(2-pyridyl)imidazoles, and 2,2′-bipyridines. Examples of other combinations of L², L³, L⁴, L⁵, and L⁶ include:
(A) Two monodentate ligands and two bidentate ligands;
(B) Four monodentate ligands and one bidentate ligand;
(C) Three monodentate ligands and one tridentate ligand;
(D) One monodentate ligand, one bidentate ligand, and one tridentate ligand;
(E) Two monodentate ligands and one tetradentate ligand; and
(F) One bidentate ligand and one tetradentate ligand.

In some embodiments, the transition metal complexes have the formula:

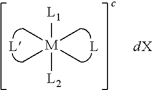

where M is a transition metal, such as for example cobalt, iron, osmium, ruthenium, nickel or vanadium; c is an integer selected from −1 to −5 or +1 to +5 indicating a positive or negative charge and X represents at least one counter ion and d is an integer from 1 to 5 representing the number of counter ions, X.

In some embodiments, one or more of L², L³, L⁴, L⁵, and L⁶ combine to form one or more bidentate ligands L′ and L. Examples of bidentate ligands include, but are not limited to, amino acids, oxalic acid, acetylacetone, diaminoalkanes, ortho-diaminoarenes, 2,2′-biimidazoles, 2,2′-bioxazoles, 2,2′-bithiazoles, 2-(2-pyridyl)imidazoles, and 2,2′-bipyridines and derivatives thereof.

In some embodiments, two or more of L², L³, L⁴, L⁵, and L⁶ combine to form one or more bidentate ligands containing at least one imidazole ring. In some instances, the bidentate ligand is a 2,2′-biimidazole having the structure:

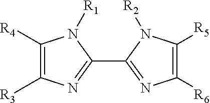

where R₁ and R₂ are substituents attached to two of the 2,2′-biimidazole nitrogens and are independently substituted or unsubstituted alkyl, alkenyl, or aryl groups. In some embodiments, R₁ and R₂ are unsubstituted C1 to C12 alkyls. In some instances, R₁ and R₂ may be unsubstituted C1 to C4 alkyls, such as for example where R₁ and R₂ are methyl.

R₃, R₄, R₅, and R₆ are substituents attached to carbon atoms of the 2,2′-biimidazole and are independently —H, —F, —Cl, —Br, —I, —NO₂, —CN, —CO₂H, —SO₃H, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxyamino, alkylthio, alkenyl, aryl, or alkyl. Alternatively, R₃ and R₄ in combination or R₅ and R₆ in combination independently form a saturated or unsaturated 5- or 6-membered ring. For example, R₃ and R₄ in combination or R₅ and R₆ in combination may form a dibenzo-2,2′-diimidazole derivative. In some embodiments, alkyl and alkoxy portions of substituents are C1 to C12. The alkyl or aryl portions of any of the substituents are in some instances, substituted with a halogen (e.g., —F, —Cl, —Br, —I), alkylamino, dialkylamino, trialkylammonium (except on aryl portions), alkoxy, alkylthio, aryl, or a reactive group. Generally, R₃, R₄, R₅, and R₆ are independently —H or unsubstituted alkyl groups. Typically, R₃, R₄, R₅, and R₆ are —H or unsubstituted C1 to C12 alkyls. In certain embodiments, R₃, R₄, R₅, and R₆ are all —H.

In some embodiments, two or more of L², L³, L⁴, L⁵, and L⁶ combine to form one or more bidentate ligands containing a 2-(2-pyridyl)-imidazole structure. In some instances, the bidentate ligand is a 2-(2-pyridyl)-imidazole having the formula:

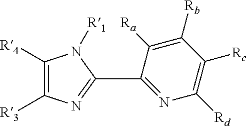

where R′₁ is a substituted or unsubstituted aryl, alkenyl, or alkyl. In some embodiments, R′₁ is a substituted or unsubstituted C1-C12 alkyl. R′₁ is in some instances, methyl or a C1-C12 alkyl that is substituted with a reactive group. By “reactive group” is meant a functional group of a molecule that is capable of reacting with another compound to couple (i.e., chemically bond) at least a portion of that other compound to the molecule. Reactive groups may include carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups, among others. The term “activated ester” is used in its conventional sense to refer to esters which have an increased reactivity and may include succinimidyl, benzotriazolyl, or aryl substituted by electron-withdrawing groups such as sulfo, nitro, cyano, or halo groups; or carboxylic acids activated by carbodiimides.

R′₃, Rₐ, R_{b}, R_{c}, and R_{d} are independently —H, —F, —Cl, —Br, —I, —NO₂, —CN, —CO₂H, —SO₃H, —NHNH₂, —SH, alkoxylcarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxylamino, alkylthio, alkenyl, aryl, or alkyl. Alternatively, R_{c} and R_{d} in combination or R′₃ and R′₄ in combination can form a saturated or unsaturated 5- or 6-membered ring. In some embodiments, the alkyl and alkoxy portions are C1 to C12. The alkyl or aryl portions of any of the substituents are in some instances, substituted with a halogen (e.g., —F, —Cl, —Br, —I), alkylamino, dialkylamino, trialkylammonium (except on aryl portions), alkoxy, alkylthio, aryl, or a reactive group. In some embodiments, R′₃, Rₐ, R_{b}, R_{c} and R_{d} are independently —H or unsubstituted alkyl groups. For example, Rₐ and R_{c} may be —H and R′₃, R′₄, R_{b}, and R_{d} may be —H or methyl.

In some embodiments, two or more of L², L³, L⁴, L⁵, and L⁶ combine to form one or more bidentate ligands containing a 2,2′-bipyridine structure. In some instances, the bidentate ligand is a 2,2′-bipyridine having the formula:

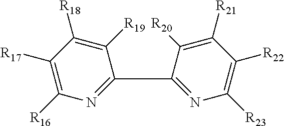

where R₁₆, R₁₇, R₁₈, R₁₉, R₂₀, R₂₁, R₂₂ and R₂₃ are independently —H, —F, —Cl, —Br, —I, —NO₂, —CN, —CO₂H, —SO₃H, —NHNH₂, —SH, aryl, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxylamino, alkylthio, alkenyl, or alkyl. In some embodiments, the alkyl and alkoxy portions are C1 to C12. The alkyl or aryl portions of any of the substituents are in some instances substituted with a halogen (e.g., —F, —Cl, —Br, —I), alkylamino, dialkylamino, trialkylammonium (except on aryl portions), alkoxy, alkylthio, aryl, or a reactive group.

Combinations of R₁₆, R₁₇, R₁₈, R₁₉, R₂₀, R₂₁, R₂₂ and R₂₃ may include R₁₆ and R₂₃ as H or methyl; R₁₇ and R₂₂ as —H or methyl; and R₁₉ and R₂₀ as —H or methyl. Alternatively, one or more adjacent pairs of substituents R₁₆ and R₁₇, and R₂₂ and R₂₃, independently form a saturated or unsaturated 5- or 6-membered ring. Another combination may include R₁₉ and R₂₀ forming a saturated or unsaturated five or six membered ring.

In some embodiments, R₁₆, R₁₇, R₁₉, R₂₀, R₂₂ and R₂₃ may be —H and R₁₈ and R₂₁ may be independently —H, alkoxy, —NH₂, alkylamino, dialkylamino, alkylthio, alkenyl, or alkyl. The alkyl or aryl portions of any of the substituents may in some instances, be substituted with a halogen (e.g., —F, —Cl, —Br, —I), alkylamino, dialkylamino, trialkylammonium (except on aryl portions), alkoxy, alkylthio, aryl, or a reactive group. For example, R₁₈ and R₂₁ may independently be —H, a C1-C6 alkyl, C1-C6 amino, C1 to C12 alkylamino, C2 to C12 dialkylamino, C1 to C12 alkylthio, or C1 to C12 alkoxy, the alkyl portions of any of the substituents may in some instance be substituted with a halogen (e.g., —F, —Cl, —Br, —I), aryl, C2 to C12 dialkylamino, C3 to C18 trialkylammonium, C1 to C6 alkoxy, C1 to C6 alkylthio or a reactive group.

In some embodiments, one or more of L², L³, L⁴, L⁵, and L⁶ are monodentate ligands. Examples of monodentate ligands include, but are not limited to, —F, —Cl, —Br, —I, —CN, —SCN, —OH, H₂O, NH₃, alkylamine, dialkylamine, trialkylamine, alkoxy or heterocyclic compounds. The alkyl or aryl portions of any of the ligands are in some instances substituted with a halogen (e.g., —F, —Cl, —Br, —I), alkylamino, dialkylamino, trialkylammonium (except on aryl portions), alkoxy, alkylthio, aryl, or a reactive group. Alkyl portions of monodentate ligands may contain 1 to 12 carbons, such as for example, 1 to 10 carbons, such as 1 to 8 carbons, including 1 to 6 carbons. In other embodiments, the monodentate ligands may be heterocyclic compounds containing at least one nitrogen, oxygen, or sulfur atom. Examples of heterocyclic monodentate ligands include, but are not limited to, imidazole, pyrazole, oxazole, thiazole, pyridine, pyrazine and derivatives thereof.

In some embodiments, heterocyclic monodentate ligands may be substituted or unsubstituted imidazoles. In some instances, the substituted imidazole ligand is an imidazole having the formula:

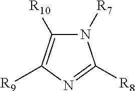

where R₇ is a substituted or unsubstituted alkyl, alkenyl, or aryl group. In some embodiments, R₇ is a substituted or unsubstituted C1 to C12 alkyl or alkenyl. The substitution of inner coordination sphere chloride anions by imidazoles does not typically cause a large shift in the redox potential in the oxidizing direction, which differs from substitution by pyridines, which typically results in a large shift in the redox potential in the oxidizing direction.

R₈, R₉ and R₁₀ are independently —H, —F, —Cl, —Br, —I, —NO₂, —CN, —CO₂H, —SO₃H, —NHNH₂, —SH, aryl, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxyamino, alkylthio, alkenyl, aryl, or alkyl. Alternatively, R₉ and R₁₀ may in combination, form a saturated or unsaturated fused 5- or 6-membered ring. Alkyl portions of the substituents may contain 1 to 12 carbons, such as for example, 1 to 10 carbons, such as 1 to 8 carbons, including 1 to 6 carbons. The alkyl or aryl portions of any of the substituents in some instances may be substituted with a halogen (e.g., —F, —Cl, —Br, —I), alkylamino, dialkylamino, trialkylammonium (except on aryl portions), alkoxy, alkylthio, aryl, or a reactive group. In some embodiments, R₈, R₉ and R₁₀ are independently —H or substituted or unsubstituted alkyl. For example, R₈, R₉ and R₁₀ are —H.

In some embodiments, heterocyclic monodentate ligands may be substituted or unsubstituted pyridines. In some instances, the substituted pyridine ligand is a pyridine having the formula:

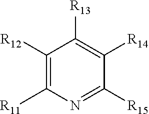

where R₁₁, R₁₂, R₁₃, R₁₄ and R₁₅ are independently —H, —F, —Cl, —Br, —I, —NO₂, —CN, —CO₂H, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxyamino, alkylthio, alkenyl, aryl, or alkyl. The alkyl or aryl portions of any of the substituents are in some instances substituted with a halogen (e.g., —F, —Cl, —Br, —I), alkylamino, dialkylamino, trialkylammonium (except for aryl portions), alkoxy, alkylthio, aryl, or a reactive group. In some embodiments, R₁₁, R₁₂, R₁₃, R₁₄ and R₁₅ are —H, methyl, C1-C2 alkoxy, C1-C2 alkylamino, C2-C4 dialkylamino, or a C1-C6 lower alkyl substituted with a reactive group.

In some embodiments, R₁₁ and R₁₅ are —H, R₁₂ and R₁₄ are independently —H or methyl, and R₁₃ is —H, C1 to C12 alkoxy, —NH₂, C1 to C12 alkylamino, C2 to C24 dialkylamino, hydrazino, C1 to C12 alkylhydrazino, hydroxylamino, C1 to C12 alkoxyamino, C1 to C12 alkylthio, or C1 to C12 alkyl. The alkyl or aryl portions of any of the substituents are in some instances, substituted with a halogen (e.g., —F, —Cl, —Br, —I), alkylamino, dialkylamino, trialkylammonium (except on aryl portions), alkoxy, alkylthio, aryl, or a reactive group.

In some embodiments, three of L², L³, L⁴, L⁵, and L⁶ combine to form a terdentate ligand.

Examples of terdentate ligands may include, but are not limited to, diethylenetriamine, 2,2′,2″-terpyridine, 2,6-bis(N-pyrazolyl)pyridine, and derivatives thereof.

In some embodiments, three or more of L², L³, L⁴, L⁵, and L⁶ combine to form a terdentate ligand containing a 2,2′,2″-terpyridine structure. In some instances, the terdentate ligand is a 2,2′,2″-terpyridine having the formula:

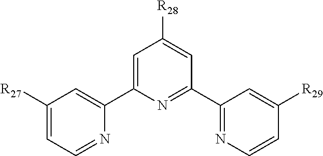

where R₂₇, R₂₈ and R₂₉ are independently —H, —F, —Cl, —Br, —I, —NO₂, —CN, —CO₂H, —SO₃H, —NHNH₂, —SH, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxylamino, alkylthio, alkenyl, aryl, or alkyl. The alkyl or aryl portions of any of the substituents are in some instances substituted with a halogen (e.g., —F, —Cl, —Br, —I), alkylamino, dialkylamino, trialkylammonium (except on aryl portions), alkoxy, alkylthio, aryl, or a reactive group. In some embodiments, the alkyl and alkoxy groups are C1 to C12. In some instances, R₂₇ and R₂₉ are —H.

In some embodiments, three of L², L³, L⁴, L⁵, and L⁶ combine to form a terdentate ligand containing a 2,6-bis(N-pyrazolyl)pyridine structure. In some instances, the terdentate ligand is a 2,6-bis(N-pyrazolyl)pyridine having the formula:

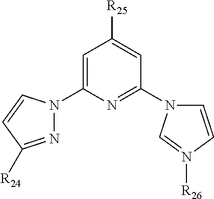

where R₂₄, R₂₅ and R₂₆ are independently —H or substituted or unsubstituted C1 to C12 alkyl. In some embodiments, R₂₄, R₂₅ and R₂₆ are independently —H or methyl. For example R₂₄ and R₂₆ are —H. Other substituents at these or other positions of the terdentate ligands are also possible.

In some embodiments, four of L², L³, L⁴, L⁵, and L⁶ combine to form a tetradentate ligand. Examples of tetradentate ligands may include, but are not limited to, triethylenetriamine, ethylenediaminediacetic acid, tetraaza macrocycles and similar compounds as well as derivatives thereof.

In certain embodiments, the transition metal complexes have the formula:

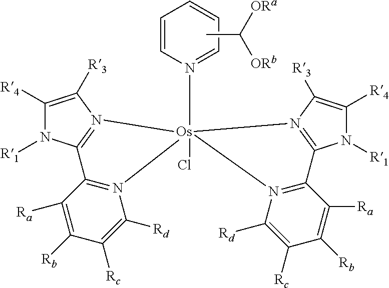

where the pyridine boronic acid ligand may have one or more boronic acid substituents, such as two or more boronic acid substituents, such as three or more boronic acid substituents, such as four of more boronic acid substituents, including five boronic acid substituents. In some instances, the boronic acid substituent may be a boronic acid (—B(OH)₂) where R^{a} and R^{b} are independently —H. In other instances, the boronic acid substituent may be a substituted boronate ester, where R^{a} and R^{b} are independently alkyl, alkenyl, aryl, alkoxy, alkoxycarbonyl, alkylaminocarbonyl, dialkylaminocarbonyl, alkylamino, dialkylamino, alkanoylamino, arylcarboxamido, hydrazino, alkylhydrazino, hydroxylamino, alkoxyamino or alkylthio.

R′₁, R′₃, R′₄, Rₐ, R_{b}, R_{c} and R_{d} are the same as described above.

In certain embodiments, R^{a} and R^{b} are —H; R′₁ is methyl; R′₃, R′₄, Rₐ, R_{b}, R_{c} and R_{d} are —H.

In some embodiments, the transition metal complexes have the formula:

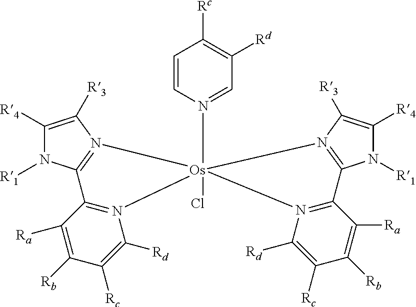

where R^{c} and R^{d} are independently —H or —B(OR¹)₂ and where R¹ is —H, alkyl, alkenyl, or aryl, alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxyamino, or alkylthio. In some embodiments, R^{c} is —H and R^{d} is —B(OH)₂. In other embodiments, R^{c} is —B(OH)₂ and R^{d} is —H.

R′₁, R′₃, R′₄, Rₐ, R_{b}, R_{c} and R_{d} are the same as described above.

In one embodiment, R^{a} is —B(OH)₂ and R^{b} is —H; R′₁ is methyl; R′₃, R′₄, Rₐ, R_{b}, R_{c} and R_{d} are —H.

In another embodiment, R^{a} is —H and R^{b} is —B(OH)₂; R′₁ is methyl; R′₃, R′₄, Rₐ, R_{b}, R_{c} and R_{d} are —H.

In some embodiments, transition metal complexes of may have the formula:

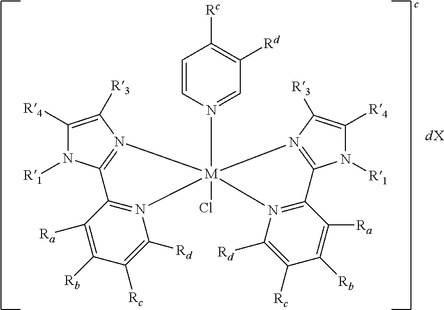

wherein c is a negative, neutral, or positive charge represented by −1 to −5, 0, or +1 to +5, inclusive, respectively;
d is a number of counter ions, X, from 1 to 5, inclusive;
M is cobalt, iron, osmium, ruthenium, nickel or vanadium;
where R^{c} and R^{d} are independently —H or —B(OR¹)₂ and where R¹ is —H, alkyl, alkenyl, or aryl, alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxyamino, or alkylthio. In some embodiments, R^{a} is —H and R^{b} is —B(OH)₂. In other embodiments, R^{a} is —B(OH)₂ and R^{b} is —H.

R′₁, R′₃, R′₄, Rₐ, R_{b}, R_{c} and R_{d} are the same as described above.

In one embodiment, R^{c} is —B(OH)₂ and R^{d} is —H; R′₁ is methyl; R′₃, R′₄, Rₐ, R_{b}, R_{c} and R_{d} are —H.

In another embodiment, R^{a} is —H and R^{b} is —B(OH)₂; R′₁ is methyl; R′₃, R′₄, Rₐ, R_{b}, R_{c} and R_{d} are —H.

The transition metal complexes can be soluble in water or other aqueous solutions, or in organic solvents. In some embodiments, the transition metal complexes can be made soluble in either aqueous or organic solvents by having an appropriate counter ion or ions, X. For example, transition metal complexes with small counter anions, such as F⁻, Cl⁻, and Br⁻, may be made more water soluble. On the other hand, transition metal complexes with bulky counter anions, such as I⁻, BF₄⁻ and PF₆⁻, may be made to be soluble in organic solvents. In embodiments, the solubility of transition metal complexes is 0.1 M (moles/liter) or more at 25° C. for a desired solvent, such as 0.15 M or more at 25° C., such as 0.2 M or more at 25° C., such as 0.25 M or more at 25° C., such as 0.3 M or more at 25° C.

The transition metal complexes can be employed as redox mediators which accept electrons from, or transfer electrons to analytes at a high rate and also exchange electrons rapidly with an electrode. Typically, the rate of self-exchange, the process in which a reduced redox mediator transfers an electron to an oxidized redox mediator, is rapid. At a defined redox mediator concentration, this provides for more rapid transport of electrons between the analyte and electrode, and thereby shortens the response time of the sensor. Additionally, the novel transition metal complex redox mediators are typically stable under ambient light and at the temperatures encountered in use, storage and transportation. The transition metal complex redox mediators, in general, do not undergo chemical change, other than oxidation and reduction, in the period of use or under the conditions of storage. In some instances, redox mediators can be configured to be activated by reacting, for example, with water or the analyte.

### Transition Metal Complexes Chemically Bonded to Polymers

Aspects of the present application also include the above described transition metal complexes chemically bonded to a polymer. By “chemically bonded” is meant that the transition metal complexes are bonded to the polymer by one or more of covalent, ionic, coordinative or non-covalent bonds. The term “non-covalent bond” is used in its conventional sense and may include hydrogen bonding, dipole-dipole interactions, charge-dipole interactions or van der Waals interactions.

In some embodiments, the transition metal complexes are chemically bonded to the polymer through one or more of carbon-carbon, carbon-nitrogen, metal-carbon covalent bonds or any combination thereof with the polymer. In some instances, transition metal complexes are chemically bonded to the polymer through one or more ligands of the mediator. For example, the transition metal complexes may be chemically bonded to the polymer through one or more of L, L′, L₁, L₂, L₃, L₄, L₅ and L₆.

Examples of transition metal complexes and polymers as well as chemical bonding between transition metal complexes and polymers are also described in U.S. Pat. Nos. 6,676,816; 6,605,200 and 6,605,201, the disclosures of which are herein incorporated by reference in their entirety.

In some embodiments, ligands of transition metal complexes are substituents of the polymeric backbone. For instance, polymeric backbones may include, for example, poly(4-vinylpyridine) and poly(N-vinylimidazole) in which the pyridine or imidazole groups, respectively, can act as monodentate ligands of the transition metal complex. In other embodiments, the transition metal complex can be the reaction product between a reactive group on a precursor polymer and a reactive group on a ligand of a precursor transition metal complex (such as where one of L, L′, L₁, L₂, L₃, L₄, L₅ and L₆ includes a reactive group as described above). Suitable precursor polymers include, for example, poly(acrylic acid) (Formula 1), styrene/maleic anhydride copolymer (Formula 2), methylvinylether/maleic anhydride copolymer (GANTREX polymer) (Formula 3), poly(vinylbenzylchloride) (Formula 4), poly(allylamine) (Formula 5), polylysine (Formula 6), carboxy-poly(vinylpyridine (Formula 7), and poly(sodium 4-styrene sulfonate) (Formula 8).

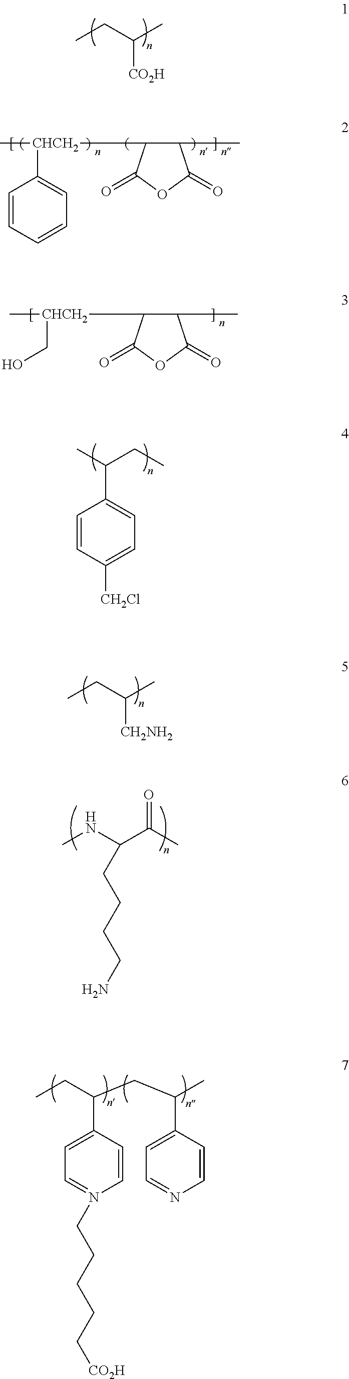

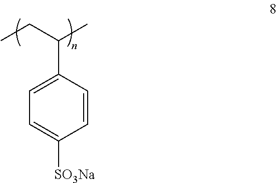

In some embodiments, the transition metal complexes are chemically bonded to the polymeric backbone through one or more spacer moieties. In some embodiments, the spacer moiety includes at least one non-cyclic functional group selected from the group consisting of —(CR^{r}R^{s})—, —O , S , C(O)O—, —S(O)₂NR^{k}—, —OC(O)NR^{m}—, —OC(S)NRⁿ, —C(O)NR^{t}—, —NR^{u}—, —CR^{v}═N—O—, —CR^{w}═NNR^{x}—, and —(SiR^{y}R^{z})—, where R^{r} and R^{s} are independently hydrogen, chlorine, fluorine, or substituted or unsubstituted alkyl, alkoxy, alkenyl, or alkynyl and R^{k}, R^{m}, Rⁿ, R^{t}, R^{v}, R^{w}, R^{x}, R^{y}, and R^{z} are independently hydrogen or substituted or unsubstituted alkyl. In some instances, the spacer includes at least four non-cyclic functional group, such as for example, at least eight non-cyclic functional groups. In other instances, the non-cyclic functional group(s) may be selected from the group consisting of —(CR^{r}R^{s})—, —O—, —S—, —C(O)O—, —S(O)₂NR^{k}—, —OC(O)NR^{m}—, —OC(S)NRⁿ, —C(O)NR^{t}—, —NR^{u}—, —CR^{v}═N—O—, —CR^{w}═NNR^{x}—, and —(SiR^{y}R^{z})—, where R^{k}, R^{m}, Rⁿ, R^{t}, R^{v}, R^{w}, R^{x}, R^{y}, and R^{z} are independently selected from the group consisting of hydrogen and substituted or unsubstituted alkyl. For example, in one embodiment, the spacer includes a 4 to 30 atom long linear segment, the linear segment having any combination of the following bonds to form the 4 to 30 atom chain of the segment: C—C, C—N, C—O, C—Si, C—S, S—N, and Si—O.

Alternatively, the transition metal complex can have reactive group(s) for immobilization or conjugation of the complexes to other substrates or carriers, examples of which include, but are not limited to, macromolecules (e.g., enzymes) and surfaces (e.g., electrode surfaces).

For reactive attachment to polymers, substrates, or other carriers, the transition metal complex precursor may include one or more reactive groups that reacts with a reactive group on the polymer, substrate, or carrier. As such a chemical bond (e.g., covalent, coordinative, ionic) is formed between the transition metal complex and the polymer, substrate or carrier. Examples of linkages are listed in Table 1, below. In certain embodiments, one of the reactive groups is an electrophile and the other reactive group is a nucleophile.

In some embodiments, polymeric transition metal complexes include a reaction product of a polymer having a polymeric backbone and a plurality of spacer moieties extending from the polymeric backbone, where at least a portion of the spacer moieties have a reactive group and a plurality of transition metal complexes.

TABLE 1
Examples of Reactive Group Linkages

| First Reactive Group | Second Reactive Group | Resulting Linkage |
|---|---|---|
| Activated ester* | Amine | Carboxamide |
| Acrylamide | Thiol | Thioether |
| Acyl azide | Amine | Carboxamide |
| Acyl halide | Amine | Carboxamide |
| Carboxylic acid | Amine | Carboxamide |
| Aldehyde or ketone | Hydrazine | Hydrazone |
| Aldehyde or ketone | Hydroxyamine | Oxime |
| Alkyl halide | Amine | Alkylamine |
| Alkyl halide | Carboxylic acid | Carboxylic ester |
| Alkyl halide | Imidazole | Imidazolium |
| Alkyl halide | Pyridine | Pyridinium |
| Alkyl halide | Alcohol/phenol | Ether |
| Alkyl halide | Thiol | Thioether |
| Alkyl sulfonate | Thiol | Thioether |
| Alkyl sulfonate | Pyridine | Pyridinium |
| Alkyl sulfonate | Imidazole | Imidazolium |
| Alkyl sulfonate | Alcohol/phenol | Ether |
| Anhydride | Alcohol/phenol | Ester |
| Anhydride | Amine | Carboxamide |
| Aziridine | Thiol | Thioether |
| Aziridine | Amine | Alkylamine |
| Aziridine | Pyridine | Pyridinium |
| Epoxide | Thiol | Thioether |
| Epoxide | Amine | Alkylamine |
| Epoxide | Pyridine | Pyridinium |
| Halotriazine | Amine | Aminotriazine |
| Halotriazine | Alcohol | Triazinyl ether |
| Imido ester | Amine | Amidine |
| Isocyanate | Amine | Urea |
| Isocyanate | Alcohol | Urethane |
| Isothiocyanate | Amine | Thiourea |
| Maleimide | Thiol | Thioether |
| Sulfonyl halide | Amine | Sulfonamide |

*Activated esters are referred to in its conventional sense to include esters of succinimidyl, benzotriazolyl, or aryl substituted by electron-withdrawing groups such as sulfo, nitro, cyano, or halo; or carboxylic acids activated by carbodiimides.

In some embodiments, the spacer includes a flexible linear chain of at least four atoms, such as at least six, such as at least eight atoms, such as at least 10 atoms, such as at least 20 atoms, and including at least 25 atoms. In some embodiments, the spacers are less than about 30 atoms. For example, the number of atoms forming the flexible linear chain may range from 1 to 25 atoms, such as 5 to 20 atoms, including 8 to 18 atoms. In some instances, two or more flexible chains are included in the spacer. The flexible chain permits the spacer to move relative to the polymeric backbone, thereby allowing mobility for the attached the transition metal complex. This is particularly useful for polymeric transition metal complexes that are employed as mediators because the movement of the transition metal complex chemically bonded to the polymer backbone by the spacer can facilitate transfer of electrons between the transition metal complex and the electrode. This enhances the rate of electron transfer and can facilitate the desired electrochemical reaction at the electrode by, for example, improving the conduction of electrons by the crosslinked and hydrated polymer on the electrode.

In addition to the chains, the spacer can contain one or more other unsaturated groups. For example, the spacer can include an unsaturated functional group such as those listed in Table 1 under the heading “Resulting Linkage”. For example, the spacer can include a heterocycle or aryl group, such as a spacer group of poly(4-vinylpyridine) or poly(N-vinylimidazole) which contain a pyridine or imidazole functional group. In these instances, the heterocycle or aryl group is positioned between the flexible chain and the polymeric backbone.

In some embodiments, transition metal complexes chemically bonded to a polymeric backbone are schematically represented by the formula:

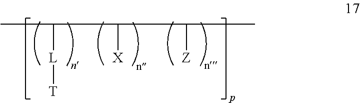

In some instances, the polymeric transition metal complex has a polymeric backbone with one or more types of pendant groups (represented by L-T, X, and Z, respectively). The individual pendant groups, L-T, X, and Z, of each polymer unit can be ordered in any configuration, where p is the number of polymer units and is an integer having a value of one or more. In some embodiments, the product of p and (n′+n″+n′″) is 5 or more, such as 10 or more, such as 25 or more, such as 50 or more, such as 75 or more, including 100 or more.

T is a transition metal complex as described above. L is a spacer moiety, as described above, and couples the transition metal complex, T, to the polymeric backbone. In some embodiments, L is absent and T is chemically bonded directly to the polymeric backbone. n′ is the number of spacer group-transition metal complex units (L-T) attached to the polymer backbone in each polymer and is an integer having a value of one or more, such as two or more, such as five or more, such as 10 or more, including 25 or more.

In some embodiments, the polymers may include one or more pendent groups, X, which do not contain a reactive substituent. The number of X pendant groups attached to the polymer backbone in each polymer unit is represented by n″ is an integer having a value of zero or more. In some embodiments, the polymeric backbones do not include any X pendant groups.

In some embodiments, the polymers may one or more pendent groups, Z , which are pendant groups substituted with a reactive substituent that includes, but is not limited to, pyridyl, imidazolyl, carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. The pendant group, Z, can be used for cross-linking the polymer backbone during, for example, polymer immobilization on a surface. The number of these pendant groups attached to the polymer backbone in each polymer unit is represented by n′″, which is an integer having a value of zero or more. In some embodiments, the polymeric backbones do not include any Z pendant groups.

The polymeric transition metal complexes can vary in molecular weight. Depending on its desired properties and application, the average molecular weight may be 5000 or more, such as 7500 or more, such as 10,000 or more, such as 25,000 or more, such as 50,000 or more, including 100,000 or more. The average molecular weight of the polymers, generally refers to the average molecular weight prior to crosslinking to form a polymeric membrane.

In some embodiments, precursor polymers used to form the polymeric transition metal complexes are poly(4-vinylpyridine) quaternized with an alkyl moiety substituted with a reactive group. For example, a precursor polymer may have the formula:

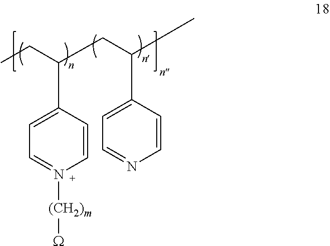

where Ω is the reactive group, m is in some instances 1 to 18, n and n′ are the average numbers of pyridinium and pyridine subunits respectively in each repeating polymer unit, ranging from 1 to 25, and n″ is the number of repeating polymer units, ranging from 1 to 1000.

In some embodiments, the transition metal complexes chemically bonded to polymeric backbones may have the formula:

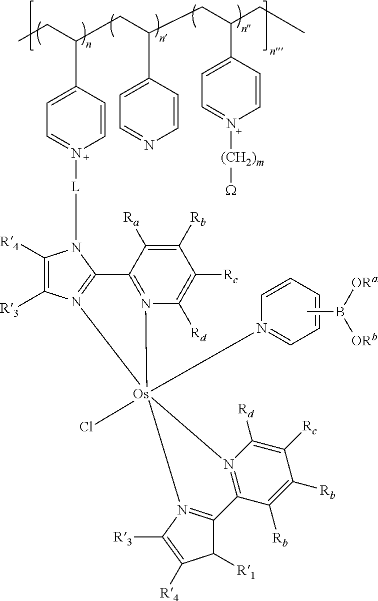

where the pyridine boronic acid may have one or more boronic acid substituents, such as two or more boronic acid substituents, such as three or more boronic acid substituents, such as four of more boronic acid substituents, including five boronic acid substituents. In some instances, the boronic acid substituent may be a boronic acid (—B(OH)₂) where R^{a} and R^{b} are independently —H. In other instances, the boronic acid substituent may be a substituted boronate ester, where R^{a} and R^{b} are independently alkyl, alkenyl, aryl, alkoxy, alkoxycarbonyl, alkylaminocarbonyl, dialkylaminocarbonyl, alkylamino, dialkylamino, alkanoylamino, arylcarboxamido, hydrazino, alkylhydrazino, hydroxylamino, alkoxyamino or alkylthio.

Ω is the reactive group, m is in some instances 1 to 18; L is the spacer, as described above, formed by the reaction of the transition metal complex to the polymer; n and n′ are the average numbers of pyridinium and pyridine subunits respectively in each repeating polymer unit, ranging from 1 to 25, and n″ is the number of repeating polymer units, ranging from 1 to 1000; R′₁, Rₐ, R_{b}, R_{c} and R_{d} are the same as described above.

In certain embodiments, R^{a} and R^{b} are —H; R′₁ is methyl; R′₃, Rₐ, R_{b}, R_{c} and R_{d} are —H.

In other embodiments, the transition metal complexes chemically bonded to polymeric backbones may have the formula:

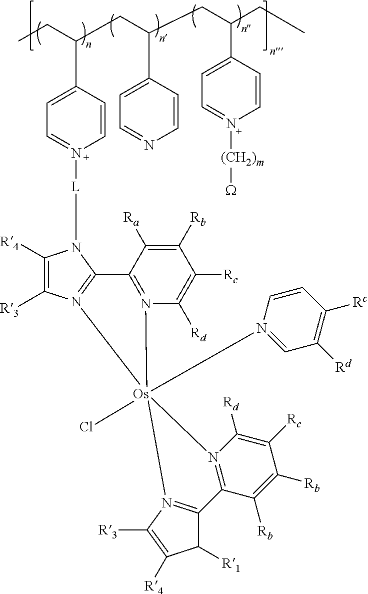

where R^{c} and R^{d} are independently —H or —B(OR¹)₂ and where R¹ is —H, alkyl, alkenyl, or aryl, alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxyamino, or alkylthio. In certain embodiments, R^{c} is —H and R^{d} is —B(OH)₂. In other embodiments, R^{c} is —B(OH)₂ and R^{d} is —H.

Ω is the reactive group, m is in some instances 1 to 18; L is the spacer, as described above, formed by the reaction of the transition metal complex to the polymer; n and n′ are the average numbers of pyridinium and pyridine subunits respectively in each repeating polymer unit, ranging from 1 to 25, and n″ is the number of repeating polymer units, ranging from 1 to 1000; R′₁, R′₃, R′₄, Rₐ, R_{b}, R_{c} and R_{d} are the same as described above.

In certain embodiments, R^{c} is —B(OH)₂ and R^{d} is —H; R′₁ is methyl; R′₃, R′₄, Rₐ, R_{b}, R_{c} and R_{d} are —H.

In another embodiment, R^{c} is —H and R^{d} is —B(OH)₂; R′₁ is methyl; R′₃, R′₄, Rₐ, R_{b}, R_{c} and R_{d} are —H.

In other embodiments, the transition metal complexes chemically bonded to polymeric backbones may have the formula:

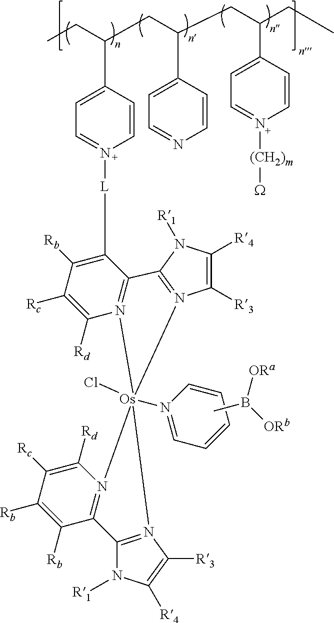

where the pyridine boronic acid may have one or more boronic acid substituents, such as two or more boronic acid substituents, such as three or more boronic acid substituents, such as four of more boronic acid substituents, including five boronic acid substituents. In some instances, the boronic acid substituent may be a boronic acid (—B(OH)₂) where R^{a} and R^{b} are independently —H. In other instances, the boronic acid substituent may be a substituted boronate ester, where R^{a} and R^{b} are independently alkyl, alkenyl, aryl, alkoxy, alkoxycarbonyl, alkylaminocarbonyl, dialkylaminocarbonyl, alkylamino, dialkylamino, alkanoylamino, arylcarboxamido, hydrazino, alkylhydrazino, hydroxylamino, alkoxyamino or alkylthio.

Ω is the reactive group, m is in some instances 1 to 18; L is the spacer, as described above, formed by the reaction of the transition metal complex to the polymer; n and n′ are the average numbers of pyridinium and pyridine subunits respectively in each repeating polymer unit, ranging from 1 to 25, and n″ is the number of repeating polymer units, ranging from 1 to 1000; R′₁, R′₃, R′₄, Rₐ, R_{b}, R_{c} and R_{d} are the same as described above.

In one embodiment, R^{a} and R^{b} are —H; R′₁ is methyl; R′₃, R′₄, Rₐ, R_{b}, R_{c} and R_{d} are —H.

In other embodiments, the transition metal complexes chemically bonded to polymeric backbones may have the formula:

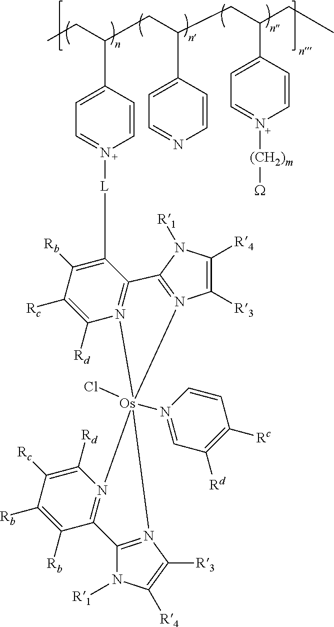

where R^{c} and R^{d} are independently —H or —B(OR¹)₂ and where R¹ is —H, alkyl, alkenyl, or aryl, alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxyamino, or alkylthio. In certain embodiments, R^{c} is —H and R^{d} is —B(OH)₂. In other embodiments, R^{c} is —B(OH)₂ and R^{d} is —H.

Ω is the reactive group, m is in some instances 1 to 18; L is the spacer, as described above, formed by the reaction of the transition metal complex to the polymer; n and n′ are the average numbers of pyridinium and pyridine subunits respectively in each repeating polymer unit, ranging from 1 to 25, and n″ is the number of repeating polymer units, ranging from 1 to 1000; R′₁, R′₃, R′₄, Rₐ, R_{b}, R_{c} and R_{d} are the same as described above.

In certain embodiments, R^{c} is —B(OH)₂ and R^{d} is —H; R′₁ is methyl; R′₃, R′₄, Rₐ, R_{b}, R_{c} and R_{d} are —H.

In another embodiment, R^{c} is —H and R^{d} is —B(OH)₂; R′₁ is methyl; R′₃, R′₄, Rₐ, R_{b}, R_{c} and R_{d} are —H.

### Crosslinking in Polymers Chemically Bonded to Transition Metal Complexes

In some embodiments, polymers which are chemically bonded to transition metal complexes may be crosslinked. The degree of crosslinking of the redox polymer can influence the transport of electrons or ions and thereby the rates of the electrochemical reactions. Excessive crosslinking of the polymer can reduce the mobility of the segments of the polymer. A reduction in segment mobility can slow the diffusion of electrons or ions through the polymer film. A reduction in the diffusivity of electrons, for example, can require a concomitant reduction in the thickness of the film on the electrode where electrons or electron vacancies are collected or delivered. The degree of crosslinking in the polymer film can thus affect the transport of electrons from, for example, analyte to transition metal complexes or an enzyme to the transition metal complexes of the polymer or between transition metal complexes and the electrode.

On the other hand, inadequate crosslinking of the polymer can result in excessive swelling of the polymer film and to the leaching of the components of the polymer film. Excessive swelling can also result in the migration of the swollen polymer into the analyzed solution, in the softening of the polymer film, in the film's susceptibility to removal by shear, or any combination of these effects.

Crosslinking can decrease the leaching of the polymer components and can improve the mechanical stability of the polymer under shear stress. For example, as disclosed in Binyamin, G. and Heller, A; Stabilization of Wired Glucose Oxidase Anodes Rotating at 1000 rpm at 37° C.; Journal of the Electrochemical Society, 146(8), 2965-2967, 1999, herein incorporated by reference, replacing a difunctional crosslinker, such as polyethylene glycol diglycidyl ether, with a trifunctional crosslinker such as N,N-diglycidyl-4-glycidyloxyaniline, for example, can reduce leaching and shear problems associated with inadequate crosslinking. In some embodiments ligands of the transition metal complexes are crosslinked to the polymer. In other embodiments, ligands of the transition metal complexes for part of the crosslinkers of the polymer.

Exemplary bifunctional, trifunctional and tetrafunctional crosslinkers include, but are not limited to:

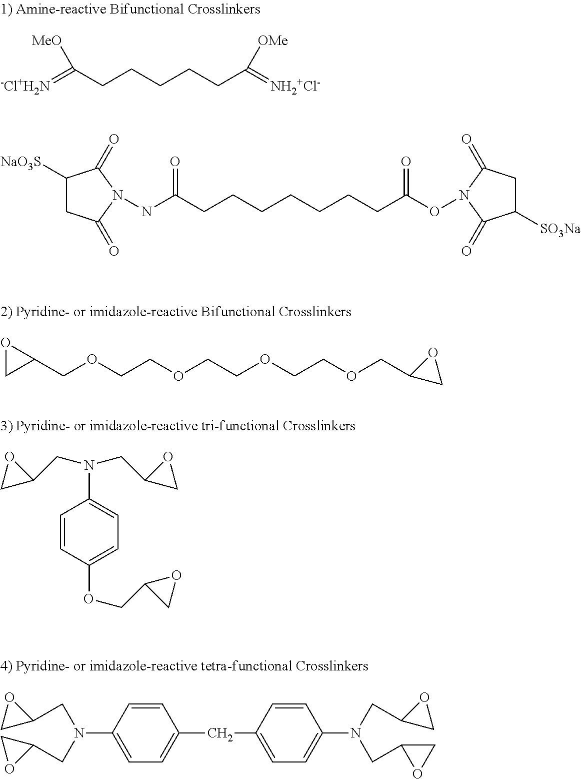

Alternatively, the number of crosslinking sites can be increased by reducing the number of transition metal complexes chemically bonded to the polymeric backbone, thus making more polymer pendant groups available for crosslinking. In some embodiments, the polymers have increased mobility of the pendant transition metal complexes, resulting from the flexibility of the pendant groups. As a result, in at least some embodiments, fewer transition metal complexes per polymer backbone are required to achieve a desired level of diffusivity of electrons and current density of analyte electrooxidation or electroreduction.

### Coordination in Transition Metal Complex Polymers

Transition metal complexes can be directly or indirectly attached to a polymeric backbone, depending on the availability and nature of the reactive groups on the transition metal complex and the polymeric backbone. For example, the pyridine groups in poly(4-vinylpyridine) or the imidazole groups in poly(N-vinylimidazole) are capable of acting as monodentate ligands and thus can be attached to a metal center directly. Alternatively, the pyridine groups in poly(4-vinylpyridine) or the imidazole groups in poly(N-vinylimidazole) can be quaternized with a substituted alkyl moiety having a suitable reactive group, such as a carboxylate function, that can be activated to form a covalent bond with a reactive group, such as an amine, of the transition metal complex (See e.g., Table 1 for a list of other types of reactive groups.).

In some embodiments, the transition metal complexes include redox centers (e.g., Os^{2+/3+}) that can be coordinated with five heterocyclic nitrogens and an additional ligand such as, for example, a chloride anion. Alternatively, the redox centers (e.g., Os^{2+/3+}) can be coordinated with six heterocyclic nitrogen atoms. Pairing of coordinating atoms can influence the potential of an electrode when the transition metal complexes are employed in electrochemical sensors, as described below.

In some embodiments, such as for the analysis of glucose, the potential at which the working electrode, coated with polymers, is poised is negative of about +250 mV vs. SCE (standard calomel electrode). In some instances, the electrode is poised negative of about +150 mV vs. SCE. Poising the electrode at these potentials reduces the interfering electrooxidation of constituents of biological fluids such as, for example, urate, ascorbate and acetaminophen. The potential can be modified by altering the ligand structure of the complex.

The redox potential of transition metal complexes chemically bonded to polymers, as described herein, is related to the potential at which the electrode is poised. Selection of transition metal complexes with a desired redox potential allows tuning of the potential at which the electrode is best poised. The redox potentials of a number of the polymers described herein are negative of about +150 mV vs. SCE and can be negative of about +50 mV vs. SCE to allow the poising of the electrode potentials negative of about +250 mV vs. SCE and in some instances negative of about +150 mV vs. SCE.

The strength of the coordination bond can influence the potential of the redox centers in the polymers. In some instances, the stronger the coordinative bond, the more positive the redox potential. A shift in the potential of a redox center resulting from a change in the coordination sphere of the transition metal can produce a labile transition metal complex. For example, when the redox potential of an Os^{2+/3+} complex is downshifted by changing the coordination sphere, the complex becomes labile. Such a labile transition metal complex may be undesirable when fashioning a polymeric transition metal complex for use as a redox mediator and can be avoided through the use of weakly coordinating multidentate or chelating heterocyclics as ligands.

### Transition Metal Complexes as Redox Mediators in Electrochemical Sensors

Aspects of the application also include employing the transition metal complexes as redox mediators in electrochemical sensors. In at least some instances, the transition metal complexes have one or more of the following characteristics: redox potentials in a particular range, the ability to exchange electrons rapidly with electrodes, the ability to rapidly transfer electrons to or rapidly accept electrons from an analyte and/or an enzyme to accelerate the kinetics of electrooxidation or electroreduction of an analyte in the presence of an enzyme or another analyte-specific redox catalyst. In some embodiments, electrochemical sensors employing transition metal complexes having at least one pyridine boronic acid ligand can determine the concentration of an analyte in a biological fluid in the absence of an analyte responsive enzyme, such as glucose oxidase or glucose dehydrogenase.

The transition metal complexes are effectively employed as redox mediators in electrochemical sensors, given their very fast kinetics. As such, when the transition metal complexes are employed in electrochemical sensors for determining the concentration of an analyte (e.g., glucose, ketone bodies, lactate) in a biological fluid, rapid electron exchange between the transition metal complexes and the working electrode in the sensor device occurs. This electron exchange is sufficiently rapid to facilitate the transfer of electrons to the working electrode that might otherwise be transferred to another electron scavenger in the system.

The electrochemical sensors may be discrete glucose monitoring devices (e.g., test strips) or may be continuous glucose monitoring devices (e.g., implantable sensors). Electrochemical sensors employing transition metal complexes, methods for determining analyte concentration in a biological fluid and methods for producing electrochemical sensors employing transition metal complexes are described, but are not limited to, those described in U.S. Pat. Nos. 5,262,035; 5,262,305; 5,320,725; 5,365,786; 5,593,852; 5,665,222; 5,972,199; and 6,143,164; 6,134,461; 6,175,752; 6,338,790; 6,592,745 and 7,225,535 the disclosures of which are herein incorporated by reference in their entirety.

Briefly, electrochemical sensors include at least one working electrode formed on a substrate. The sensor may also include at least one counter electrode (or counter/reference electrode) and/or at least one reference electrode. The counter electrode and/or reference electrode may be formed on the substrate or may be separate units. For example, the counter electrode and/or reference electrode may be formed on a second substrate which is also implanted in the patient or, for some embodiments of the implantable sensors, the counter electrode and/or reference electrode may be placed on the skin of the patient with the working electrode or electrodes being implanted into the patient. The use of an on-the-skin counter and/or reference electrode with an implantable working electrode is described in U.S. Pat. No. 5,593,852, the disclosure of which is herein incorporated by reference.

The working electrode(s) may be formed using conductive traces disposed on the substrate. The counter electrode and/or reference electrode, as well as other optional portions of the sensor may also be formed using conductive traces disposed on the substrate. These conductive traces may be formed over a smooth surface of the substrate or within channels formed by, for example, embossing, indenting or otherwise creating a depression in the substrate.

The working electrode may be formed from a molded carbon fiber composite or may consist of an inert non-conducting base material, such as polyester, upon which a suitable conducting layer is deposited. The conducting layer has relatively low electrical resistance and is typically electrochemically inert over the potential range of the sensor during operation. Conducting layers may include gold, carbon, platinum, ruthenium dioxide, palladium, and conductive epoxies, such as, for example, ECCOCOAT CT5079-3 Carbon-Filled Conductive Epoxy Coating (available from W. R. Grace Company, Woburn, Mass.) as well as other non-corroding materials known to those skilled in the art. The working electrode (e.g., the conducting layer) is deposited on the surface of the inert material by methods such as vapor deposition or printing.

A tab may be provided on the end of the working electrode for easy connection of the electrode to external electronics such as a voltage source or current measuring equipment. Other known methods or structures (such as contact pads) may be used to connect the working electrode to the external electronics.

Counter electrode and/or reference electrodes may be constructed in a manner similar to working electrode. The counter and/or reference electrodes may be replaced with a single counter/reference electrode. Alternatively, a separate reference electrode may be provided in contact with the sample chamber. Suitable materials for the counter, reference, or counter/reference electrode include Ag/AgCl or Ag/AgBr printed on a non-conducting base material or silver chloride on a silver metal base. The same materials and methods may be used to make the counter electrode as are available for constructing the working electrode, although different materials and methods may also be used.

Any number of different electrode configurations may be employed. In one embodiment, the electrochemical sensor includes two working electrodes and one counter electrode, which also functions as a reference electrode (i.e., counter/reference electrode). In another embodiment, the sensor includes one working electrode, one counter electrode, and one reference electrode. In some instances, all of the electrodes are positioned on the same side of the substrate.

Alternatively, one or more of the electrodes may be formed on an opposing side of the substrate, such as in a face-to-face configuration. This may be convenient if the electrodes are formed using two different types of conductive material (e.g., carbon and silver/silver chloride). Then, at least in some embodiments, only one type of conductive material needs to be applied to each side of the substrate, thereby reducing the number of steps in the manufacturing process and/or easing the registration constraints in the process. For example, if the working electrode is formed using a carbon-based conductive material and the reference or counter/reference electrode is formed using a silver/silver chloride conductive material, then the working electrode and reference or counter/reference electrode may be formed on opposing sides of the substrate for ease of manufacture.

In yet another embodiment, two working electrodes and one counter electrode are formed on one side (i.e., face) of the substrate and one reference electrode are formed on an opposing side (i.e., opposite face) of the substrate.

In some embodiments, electrochemical sensors include a sensing layer which contain the instant transition metal complexes disposed on top or in proximity (e.g., in a solution surrounding) to the working electrode. By “in proximity” is meant that the transition metal complexes of the sensing layer are in electrical communication with the electrode. In some instances, the transition metal complexes of the sensing layer are in physical contact with the electrode. The transition metal complexes transfer electrons between the working electrode and an analyte. The components of the sensing layer may be in a fluid or gel that is proximate to or in contact with the working electrode. Alternatively, the components of the sensing layer may be disposed in a polymer (as described above for transition metal complexes chemically bonded to a polymer) or sol-gel matrix that is proximate to or on the working electrode. Sensing layers, their configuration and components may include, but are not limited to, those described in U.S. Pat. Nos. 6,175,752 and 6,592,745, the disclosures of which are herein incorporated by reference.

The transition metal complexes described herein are particularly effective redox mediators in electrochemical sensing applications, due to their enhanced ability to collect charge at the working electrode, which in turn enhances the sensitivity of the sensor to the concentration of the measured analyte. For example, an oxidized form of the redox mediator interacts with a source of electrons, thereby receiving an electron and becoming reduced. The reduced mediator transfers electrons to the electrode, which oxidizes the redox mediator and creating a current at the working electrode. Transition metal complexes described herein possess desirable redox potentials in a range at which the electron-transfer kinetics is optimized, or maximized, and the effect of common interfering species present in biofluid is minimized.

In certain embodiments, the sensing layer does not require the presence of any catalyst or enzyme. As such, the electrochemical sensors are capable and configured to determine the concentration of an analyte (e.g., glucose, ketone bodies, lactate) in the absence of an analyte responsive enzyme. Electrochemical sensors employing pyridine boronic acid transition metal (e.g., Os) complexes as redox mediators can be used to determine the concentration of glucose in a biological fluid in the absence of an enzyme. By eliminating the need for an enzyme to catalyze the electrolysis of the analyte, the electrochemical sensors demonstrate greater stability, storage capability as well as fewer interference and potential degradation of catalysts in the sensing layer.

In other embodiments, the sensing layer consists of polymeric transition metal complexes. As such, the electrochemical sensors include only pyridine boronic acid transition metal complexes, in the absence of an analyte responsive enzyme, disposed on the surface of the working electrode.

In other embodiments, an enzyme may be included in the sensing layer. As such, the redox mediator transfers electrons between the analyte and the working electrode (via an enzyme) in an enzyme-catalyzed reaction of the analyte. In these embodiments, transition metal complexes described herein can be used as a redox mediator in combination with an analyte responsive enzyme to electrooxidize or electroreduce the analyte or a compound derived of the analyte, for example by hydrolysis of the analyte. The redox potentials of the redox mediators may be more positive (i.e. more oxidizing) than the redox potentials of the enzymes when the analyte is electrooxidized and more negative when the analyte is electroreduced. For example, the redox potentials of some transition metal complexes employed as redox mediators for electrooxidizing glucose with glucose oxidase or glucose dehydrogenase is between about −200 mV and +200 mV versus a Ag/AgCl reference electrode, such as between about −100 mV and about +100 mV versus a Ag/AgCl reference electrode. Sensing layers which employ an analyte responsive enzyme may include, but are not limited to, those described in U.S. Pat. Nos. 6,175,752 and 6,592,745, the disclosures of which are herein incorporated by reference.

In some embodiments, the electrochemical sensors may further include a biocompatible layer. The biocompatible layer may be formed over at least a portion of the sensor (e.g., the portion of the sensor that is subcutaneously inserted into a host). The biocompatible layer, in some embodiments, prevents the penetration of large biomolecules into the electrodes. This is accomplished by using a biocompatible layer having a pore size that is smaller than the biomolecules that are to be excluded. Such biomolecules may foul the electrodes and/or the sensing layer thereby reducing the effectiveness of the sensor and altering the expected signal amplitude for a given analyte concentration. The fouling of the working electrode(s) may also decrease the effective life of the sensor. In other embodiments, the biocompatible layer may prevent protein adhesion to the sensor, formation of blood clots, and other undesirable interactions between the sensor and body, such as barrier cell layer formation or fibrotic tissue formation.

In some embodiments, the electrochemical sensors may further include an interferent-eliminating layer. The interferent-eliminating layer may be incorporated in the biocompatible layer or in a mass transport limiting layer (described below) or may be a separate layer. By “interferents” is meant molecules or other species that are electroreduced or electrooxidized at the electrode, either directly or via an electron transfer agent, to produce a false signal. In one embodiment, a film or membrane prevents the penetration of one or more interferents into the region around the working electrodes. In some instances, this type of interferent-eliminating layer is much less permeable to one or more of the interferents than to the analyte.

In some embodiments, the electrochemical sensors may further include a mass transport limiting layer. Mass transport limiting layers serve as a diffusion-limiting barrier to reduce the rate of mass transport of the analyte, for example, glucose or lactate, into the region around the working electrodes. By limiting the diffusion of the analyte, the steady state concentration of the analyte in the proximity of the working electrode (which is proportional to the concentration of the analyte in the body or sample fluid) can be reduced. This extends the upper range of analyte concentrations that can still be accurately measured and may also expand the range in which the current increases approximately linearly with the level of the analyte. Biocompatible, interferent eliminating and mass transport-limiting layers may include, but are not limited to those described in U.S. Pat. Nos. 6,932,894 and 6,175,752, the disclosures of which are herein incorporated by reference in their entirety.

In some embodiments, the electrochemical sensors include a sample chamber which may be defined by a combination of the electrodes (e.g., working, counter, reference) an inert base, and a spacer element. A measurement zone is contained within this sample chamber and is the region of the sample chamber that contains only that portion of the sample that is interrogated during the analyte assay. In some embodiments, the sample chamber is a space between the working electrode and counter electrode and/or the inert base. In some embodiments, the sample chamber has a volume that is not greater than about 1 μL, such as not greater than about 0.5 μL, including not greater than about 0.25 μL. In some instances, the measurement zone has a volume that is approximately equal to the volume of the sample chamber. For example, the measurement zone may be about 5% of the sample chamber of more, such as about 10% of the sample chamber of more, such as about 25% of the sample chamber of more, such as about 50% of the sample chamber of more, such as about 75% of the sample chamber or more, such as about 90% of the sample chamber or more, including about 100% of the sample chamber.

Sample chambers, their configuration, size and methods for producing them may include, but are not limited to, those described in U.S. Pat. No. 6,592,745, the disclosure of which is herein incorporated by reference in its entirety.

### Electrochemical Sensors

Embodiments of the present disclosure relate to methods and devices for detecting at least one analyte, including glucose, in body fluid. Embodiments relate to the continuous and/or automatic in vivo monitoring of the level of one or more analytes using a continuous analyte monitoring system that includes an analyte sensor at least a portion of which is to be positioned beneath a skin surface of a user for a period of time and/or the discrete monitoring of one or more analytes using an in vitro blood glucose (“BG”) meter and an analyte test strip. Embodiments include combined or combinable devices, systems and methods and/or transferring data between an in vivo continuous system and an in vivo system. In some embodiments, the systems, or at least a portion of the systems, are integrated into a single unit.

A sensor as described herein may be an in vivo sensor or an in vitro sensor (i.e., a discrete monitoring test strip). Such a sensor can be formed on a substrate, e.g., a substantially planar substrate. In certain embodiments, the sensor is a wire, e.g., a working electrode wire inner portion with one or more other electrodes associated (e.g., on, including wrapped around) therewith. The sensor may also include at least one counter electrode (or counter/reference electrode) and/or at least one reference electrode or at least one reference/counter electrode.

Accordingly, embodiments include analyte monitoring devices and systems that include an analyte sensor at least a portion of which is positionable beneath the skin surface of the user for the in vivo detection of an analyte, including glucose, lactate, and the like, in a body fluid. Embodiments include wholly implantable analyte sensors and analyte sensors in which only a portion of the sensor is positioned under the skin and a portion of the sensor resides above the skin, e.g., for contact to a sensor control unit (which may include a transmitter), a receiver/display unit, transceiver, processor, etc. The sensor may be, for example, subcutaneously positionable in a user for the continuous or periodic monitoring of a level of an analyte in the user's interstitial fluid. For the purposes of this description, continuous monitoring and periodic monitoring will be used interchangeably, unless noted otherwise. The sensor response may be correlated and/or converted to analyte levels in blood or other fluids. In certain embodiments, an analyte sensor may be positioned in contact with interstitial fluid to detect the level of glucose, which detected glucose may be used to infer the glucose level in the user's bloodstream. Analyte sensors may be insertable into a vein, artery, or other portion of the body containing fluid. Embodiments of the analyte sensors may be configured for monitoring the level of the analyte over a time period which may range from seconds, minutes, hours, days, weeks, to months, or longer.

In certain embodiments, the analyte sensors, such as glucose sensors, are capable of in vivo detection of an analyte for one hour or more, e.g., a few hours or more, e.g., a few days or more, e.g., three or more days, e.g., five days or more, e.g., seven days or more, e.g., several weeks or more, or one month or more. Future analyte levels may be predicted based on information obtained, e.g., the current analyte level at time t₀, the rate of change of the analyte, etc. Predictive alarms may notify the user of a predicted analyte levels that may be of concern in advance of the user's analyte level reaching the future predicted analyte level. This provides the user an opportunity to take corrective action.

In an electrochemical embodiment, the sensor is placed, transcutaneously, for example, into a subcutaneous site such that subcutaneous fluid of the site comes into contact with the sensor. In other in vivo embodiments, placement of at least a portion of the sensor may be in a blood vessel. The sensor operates to electrolyze an analyte of interest in the subcutaneous fluid or blood such that a current is generated between the working electrode and the counter electrode. A value for the current associated with the working electrode is determined. If multiple working electrodes are used, current values from each of the working electrodes may be determined. A microprocessor may be used to collect these periodically determined current values or to further process these values.

If an analyte concentration is successfully determined, it may be displayed, stored, transmitted, and/or otherwise processed to provide useful information. By way of example, raw signal or analyte concentrations may be used as a basis for determining a rate of change in analyte concentration, which should not change at a rate greater than a predetermined threshold amount. If the rate of change of analyte concentration exceeds the predefined threshold, an indication maybe displayed or otherwise transmitted to indicate this fact. In certain embodiments, an alarm is activated to alert a user if the rate of change of analyte concentration exceeds the predefined threshold.

As demonstrated herein, the methods of the present disclosure are useful in connection with a device that is used to measure or monitor an analyte (e.g., glucose), such as any such device described herein. These methods may also be used in connection with a device that is used to measure or monitor another analyte (e.g., ketones, ketone bodies, HbA1c, and the like), including oxygen, carbon dioxide, proteins, drugs, or another moiety of interest, for example, or any combination thereof, found in bodily fluid, including subcutaneous fluid, dermal fluid (sweat, tears, and the like), interstitial fluid, or other bodily fluid of interest, for example, or any combination thereof. In general, the device is in good contact, such as thorough and substantially continuous contact, with the bodily fluid.

According to embodiments of the present disclosure, the measurement sensor is one suited for electrochemical measurement of analyte concentration, for example glucose concentration, in a bodily fluid. In these embodiments, the measurement sensor includes at least a working electrode and a counter electrode. Other embodiments may further include a reference electrode. The working electrode is typically associated with a glucose-responsive enzyme. A mediator may also be included. In certain embodiments, hydrogen peroxide, which may be characterized as a mediator, is produced by a reaction of the sensor and may be used to infer the concentration of glucose. In some embodiments, a mediator is added to the sensor by a manufacturer, i.e., is included with the sensor prior to use. The redox mediator may be disposed relative to the working electrode and is capable of transferring electrons between a compound and a working electrode, either directly or indirectly. The redox mediator may be, for example, immobilized on the working electrode, e.g., entrapped on a surface or chemically bound to a surface.

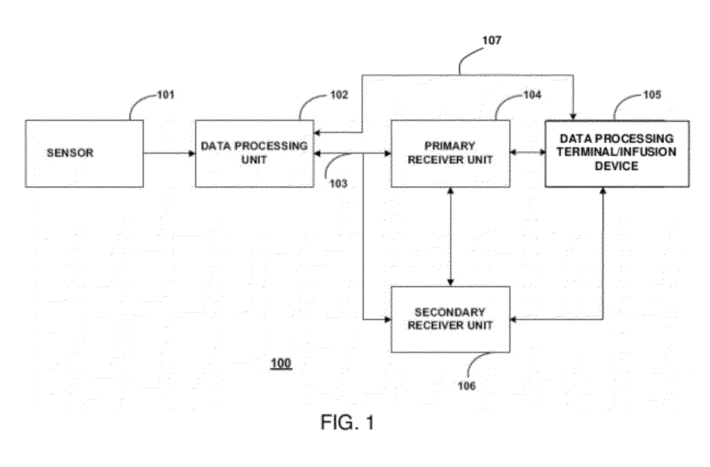

FIG. 1 shows a data monitoring and management system such as, for example, an analyte (e.g., glucose) monitoring system 100 in accordance with certain embodiments. Aspects of the subject disclosure are further described primarily with respect to glucose monitoring devices and systems, and methods of glucose detection, for convenience only and such description is in no way intended to limit the scope of the embodiments. It is to be understood that the analyte monitoring system may be configured to monitor a variety of analytes at the same time or at different times.

Analytes that may be monitored include, but are not limited to, acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

The analyte monitoring system 100 includes an analyte sensor 101, a data processing unit 102 connectable to the sensor 101, and a primary receiver unit 104. In some instances, the primary receiver unit 104 is configured to communicate with the data processing unit 102 via a communication link 103. In certain embodiments, the primary receiver unit 104 may be further configured to transmit data to a data processing terminal 105 to evaluate or otherwise process or format data received by the primary receiver unit 104. The data processing terminal 105 may be configured to receive data directly from the data processing unit 102 via a communication link 107, which may optionally be configured for bi-directional communication. Further, the data processing unit 102 may include a transmitter or a transceiver to transmit and/or receive data to and/or from the primary receiver unit 104 and/or the data processing terminal 105 and/or optionally a secondary receiver unit 106.

Also shown in FIG. 1 is an optional secondary receiver unit 106 which is operatively coupled to the communication link 103 and configured to receive data transmitted from the data processing unit 102. The secondary receiver unit 106 may be configured to communicate with the primary receiver unit 104, as well as the data processing terminal 105. In certain embodiments, the secondary receiver unit 106 may be configured for bi-directional wireless communication with each of the primary receiver unit 104 and the data processing terminal 105. As discussed in further detail below, in some instances, the secondary receiver unit 106 may be a de-featured receiver as compared to the primary receiver unit 104, for instance, the secondary receiver unit 106 may include a limited or minimal number of functions and features as compared with the primary receiver unit 104. As such, the secondary receiver unit 106 may include a smaller (in one or more, including all, dimensions), compact housing or embodied in a device including a wrist watch, arm band, PDA, mp3 player, cell phone, etc., for example. Alternatively, the secondary receiver unit 106 may be configured with the same or substantially similar functions and features as the primary receiver unit 104. The secondary receiver unit 106 may include a docking portion configured to mate with a docking cradle unit for placement by, e.g., the bedside for night time monitoring, and/or a bi-directional communication device. A docking cradle may recharge a power supply.

Only one analyte sensor 101, data processing unit 102 and data processing terminal 105 are shown in the embodiment of the analyte monitoring system 100 illustrated in FIG. 1. However, it will be appreciated by one of ordinary skill in the art that the analyte monitoring system 100 may include more than one sensor 101 and/or more than one data processing unit 102, and/or more than one data processing terminal 105. Multiple sensors may be positioned in a user for analyte monitoring at the same or different times. In certain embodiments, analyte information obtained by a first sensor positioned in a user may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring system 100 may be a continuous monitoring system, or semi-continuous, or a discrete monitoring system. In a multi-component environment, each component may be configured to be uniquely identified by one or more of the other components in the system so that communication conflict may be readily resolved between the various components within the analyte monitoring system 100. For example, unique IDs, communication channels, and the like, may be used.

In certain embodiments, the sensor 101 is physically positioned in or on the body of a user whose analyte level is being monitored. The sensor 101 may be configured to at least periodically sample the analyte level of the user and convert the sampled analyte level into a corresponding signal for transmission by the data processing unit 102. The data processing unit 102 is coupleable to the sensor 101 so that both devices are positioned in or on the user's body, with at least a portion of the analyte sensor 101 positioned transcutaneously. The data processing unit may include a fixation element, such as an adhesive or the like, to secure it to the user's body. A mount (not shown) attachable to the user and mateable with the data processing unit 102 may be used. For example, a mount may include an adhesive surface. The data processing unit 102 performs data processing functions, where such functions may include, but are not limited to, filtering and encoding of data signals, each of which corresponds to a sampled analyte level of the user, for transmission to the primary receiver unit 104 via the communication link 103. In some embodiments, the sensor 101 or the data processing unit 102 or a combined sensor/data processing unit may be wholly implantable under the skin surface of the user.

In certain embodiments, the primary receiver unit 104 may include an analog interface section including an RF receiver and an antenna that is configured to communicate with the data processing unit 102 via the communication link 103, and a data processing section for processing the received data from the data processing unit 102 including data decoding, error detection and correction, data clock generation, data bit recovery, etc., or any combination thereof.

In operation, the primary receiver unit 104 in certain embodiments is configured to synchronize with the data processing unit 102 to uniquely identify the data processing unit 102, based on, for example, an identification information of the data processing unit 102, and thereafter, to periodically receive signals transmitted from the data processing unit 102 associated with the monitored analyte levels detected by the sensor 101.

Referring again to FIG. 1, the data processing terminal 105 may include a personal computer, a portable computer including a laptop or a handheld device (e.g., a personal digital assistant (PDA), a telephone including a cellular phone (e.g., a multimedia and Internet-enabled mobile phone including an iPhone™, a Blackberry®, or similar phone), an mp3 player (e.g., an iPOD™, etc.), a pager, and the like), and/or a drug delivery device (e.g., an infusion device), each of which may be configured for data communication with the receiver via a wired or a wireless connection. Additionally, the data processing terminal 105 may further be connected to a data network (not shown) for storing, retrieving, updating, and/or analyzing data corresponding to the detected analyte level of the user.

The data processing terminal 105 may include a drug delivery device (e.g., an infusion device) such as an insulin infusion pump or the like, which may be configured to administer a drug (e.g., insulin) to the user, and which may be configured to communicate with the primary receiver unit 104 for receiving, among others, the measured analyte level. Alternatively, the primary receiver unit 104 may be configured to integrate an infusion device therein so that the primary receiver unit 104 is configured to administer an appropriate drug (e.g., insulin) to users, for example, for administering and modifying basal profiles, as well as for determining appropriate boluses for administration based on, among others, the detected analyte levels received from the data processing unit 102. An infusion device may be an external device or an internal device, such as a device wholly implantable in a user.

In certain embodiments, the data processing terminal 105, which may include an infusion device, e.g., an insulin pump, may be configured to receive the analyte signals from the data processing unit 102, and thus, incorporate the functions of the primary receiver unit 104 including data processing for managing the user's insulin therapy and analyte monitoring. In certain embodiments, the communication link 103, as well as one or more of the other communication interfaces shown in FIG. 1, may use one or more wireless communication protocols, such as, but not limited to: an RF communication protocol, an infrared communication protocol, a Bluetooth enabled communication protocol, an 802.11x wireless communication protocol, or an equivalent wireless communication protocol which would allow secure, wireless communication of several units (for example, per Health Insurance Portability and Accountability Act (HIPPA) requirements), while avoiding potential data collision and interference.

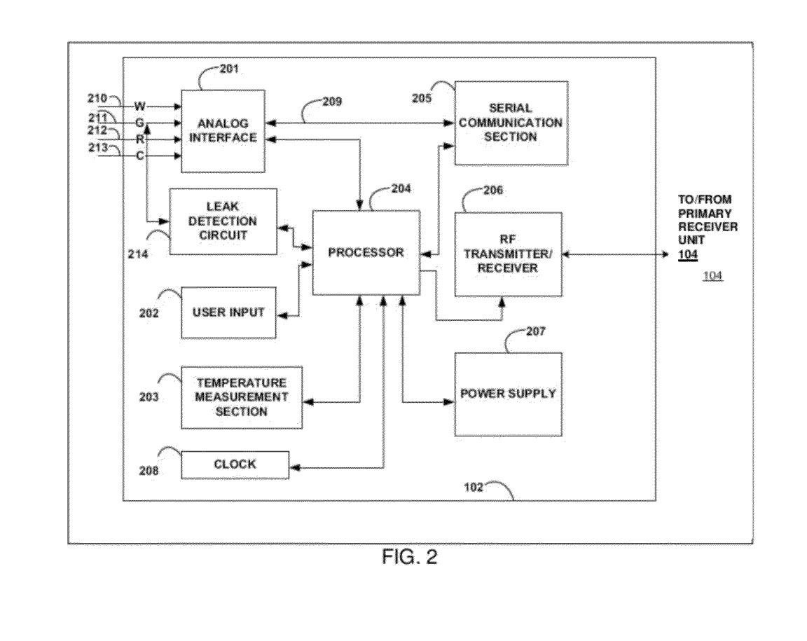

FIG. 2 shows a block diagram of an embodiment of a data processing unit 102 of the analyte monitoring system shown in FIG. 1. User input and/or interface components may be included or a data processing unit may be free of user input and/or interface components. In certain embodiments, one or more application-specific integrated circuits (ASIC) may be used to implement one or more functions or routines associated with the operations of the data processing unit (and/or receiver unit) using for example one or more state machines and buffers.

As can be seen in the embodiment of FIG. 2, the analyte sensor 101 (FIG. 1) includes four contacts, three of which are electrodes: a work electrode (W) 210, a reference electrode (R) 212, and a counter electrode (C) 213, each operatively coupled to the analog interface 201 of the data processing unit 102. This embodiment also shows an optional guard contact (G) 211. Fewer or greater electrodes may be employed. For example, the counter and reference electrode functions may be served by a single counter/reference electrode. In some cases, there may be more than one working electrode and/or reference electrode and/or counter electrode, etc.

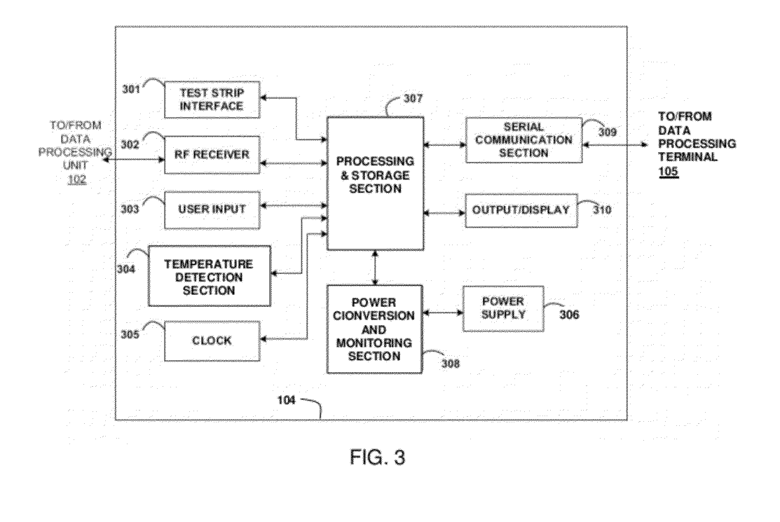

FIG. 3 is a block diagram of an embodiment of a receiver/monitor unit such as the primary receiver unit 104 of the analyte monitoring system shown in FIG. 1. The primary receiver unit 104 includes one or more of: a test strip interface 301, an RF receiver 302, a user input 303, an optional temperature detection section 304, and a clock 305, each of which is operatively coupled to a processing and storage section 307. The primary receiver unit 104 also includes a power supply 306 operatively coupled to a power conversion and monitoring section 308. Further, the power conversion and monitoring section 308 is also coupled to the processing and storage section 307. Moreover, also shown are a receiver serial communication section 309, and an output 310, each operatively coupled to the processing and storage section 307. The primary receiver unit 104 may include user input and/or interface components or may be free of user input and/or interface components.

In certain embodiments, the test strip interface 301 includes an analyte testing portion (e.g., a glucose level testing portion) to receive a blood (or other body fluid sample) analyte test or information related thereto. For example, the test strip interface 301 may include a test strip port to receive a test strip (e.g., a glucose test strip). The device may determine the analyte level of the test strip, and optionally display (or otherwise notice) the analyte level on the output 310 of the primary receiver unit 104. Any suitable test strip may be employed, e.g., test strips that only require a very small amount (e.g., 3 microliters or less, e.g., 1 microliter or less, e.g., 0.5 microliters or less, e.g., 0.1 microliters or less), of applied sample to the strip in order to obtain accurate glucose information. Embodiments of test strips include, e.g., Freestyle® blood glucose test strips from Abbott Diabetes Care, Inc. (Alameda, Calif.). Glucose information obtained by an in vitro glucose testing device may be used for a variety of purposes, computations, etc. For example, the information may be used to calibrate sensor 101, confirm results of sensor 101 to increase the confidence thereof (e.g., in instances in which information obtained by sensor 101 is employed in therapy related decisions), etc.

In further embodiments, the data processing unit 102 and/or the primary receiver unit 104 and/or the secondary receiver unit 106, and/or the data processing terminal/infusion device 105 may be configured to receive the analyte value wirelessly over a communication link from, for example, a blood glucose meter. In further embodiments, a user manipulating or using the analyte monitoring system 100 (FIG. 1) may manually input the analyte value using, for example, a user interface (for example, a keyboard, keypad, voice commands, and the like) incorporated in one or more of the data processing unit 102, the primary receiver unit 104, secondary receiver unit 106, or the data processing terminal/infusion device 105.

Additional detailed descriptions are provided in U.S. Pat. Nos. 5,262,035; 5,264,104; 5,262,305; 5,320,715; 5,593,852; 6,175,752; 6,650,471; 6,746,582, and 7,811,231, each of which is incorporated herein by reference in their entirety.

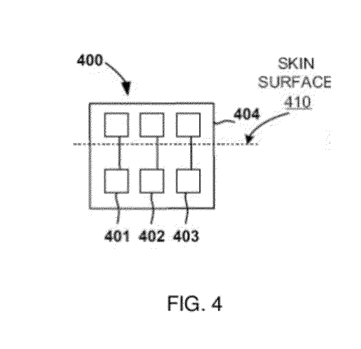

FIG. 4 schematically shows an embodiment of an analyte sensor 400 in accordance with the embodiments of the present disclosure. This sensor embodiment includes electrodes 401, 402 and 403 on a base 404. Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching, and the like. Materials include, but are not limited to, any one or more of aluminum, carbon (including graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

The analyte sensor 400 may be wholly implantable in a user or may be configured so that only a portion is positioned within (internal) a user and another portion outside (external) a user. For example, the sensor 400 may include a first portion positionable above a surface of the skin 410, and a second portion positioned below the surface of the skin. In such embodiments, the external portion may include contacts (connected to respective electrodes of the second portion by traces) to connect to another device also external to the user such as a transmitter unit. While the embodiment of FIG. 4 shows three electrodes side-by-side on the same surface of base 404, other configurations are contemplated, e.g., fewer or greater electrodes, some or all electrodes on different surfaces of the base or present on another base, some or all electrodes stacked together, electrodes of differing materials and dimensions, etc.

FIG. 5A shows a perspective view of an embodiment of an analyte sensor 500 having a first portion (which in this embodiment may be characterized as a major portion) positionable above a surface of the skin 510, and a second portion (which in this embodiment may be characterized as a minor portion) that includes an insertion tip 530 positionable below the surface of the skin, e.g., penetrating through the skin and into, e.g., the subcutaneous space 520, in contact with the user's biofluid, such as interstitial fluid. Contact portions of a working electrode 511, a reference electrode 512, and a counter electrode 513 are positioned on the first portion of the sensor 500 situated above the skin surface 510. A working electrode 501, a reference electrode 502, and a counter electrode 503 are shown at the second portion of the sensor 500 and particularly at the insertion tip 530. Traces may be provided from the electrodes at the tip to the contact, as shown in FIG. 5A. It is to be understood that greater or fewer electrodes may be provided on a sensor. For example, a sensor may include more than one working electrode and/or the counter and reference electrodes may be a single counter/reference electrode, etc.

FIG. 5B shows a cross sectional view of a portion of the sensor 500 of FIG. 5A. The electrodes 501, 502 and 503, of the sensor 500 as well as the substrate and the dielectric layers are provided in a layered configuration or construction. For example, as shown in FIG. 5B, in one embodiment, the sensor 500 (such as the analyte sensor unit 101 of FIG. 1), includes a substrate layer 504, and a first conducting layer 501 such as carbon, gold, etc., disposed on at least a portion of the substrate layer 504, and which may provide the working electrode. Also shown disposed on at least a portion of the first conducting layer 501 is a sensing layer 508.

A first insulation layer 505, such as a first dielectric layer in certain embodiments, is disposed or layered on at least a portion of the first conducting layer 501, and further, a second conducting layer 509 may be disposed or stacked on top of at least a portion of the first insulation layer (or dielectric layer) 505. As shown in FIG. 5B, the second conducting layer 509 may provide the reference electrode 502, as described herein having an extended lifetime, which includes a layer of redox polymer as described herein.

A second insulation layer 506, such as a second dielectric layer in certain embodiments, may be disposed or layered on at least a portion of the second conducting layer 509. Further, a third conducting layer 503 may be disposed on at least a portion of the second insulation layer 506 and may provide the counter electrode 503. Finally, a third insulation layer 507 may be disposed or layered on at least a portion of the third conducting layer 503. In this manner, the sensor 500 may be layered such that at least a portion of each of the conducting layers is separated by a respective insulation layer (for example, a dielectric layer). The embodiments of FIGS. 5A and 5B show the layers having different lengths. In certain instances, some or all of the layers may have the same or different lengths and/or widths.

In certain embodiments, some or all of the electrodes 501, 502, 503 may be provided on the same side of the substrate 504 in the layered construction as described above, or alternatively, may be provided in a co-planar manner such that two or more electrodes may be positioned on the same plane (e.g., side-by side (e.g., parallel) or angled relative to each other) on the substrate 504. For example, co-planar electrodes may include a suitable spacing therebetween and/or include a dielectric material or insulation material disposed between the conducting layers/electrodes. Furthermore, in certain embodiments, one or more of the electrodes 501, 502, 503 may be disposed on opposing sides of the substrate 504. In such embodiments, contact pads may be one the same or different sides of the substrate. For example, an electrode may be on a first side and its respective contact may be on a second side, e.g., a trace connecting the electrode and the contact may traverse through the substrate.

As noted above, analyte sensors may include an analyte-responsive enzyme to provide a sensing component or sensing layer. Some analytes, such as oxygen, can be directly electrooxidized or electroreduced on a sensor, and more specifically at least on a working electrode of a sensor. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing layer (see for example sensing layer 508 of FIG. 5B) proximate to or on a surface of a working electrode. In many embodiments, a sensing layer is formed near or on only a small portion of at least a working electrode.

The sensing layer includes one or more components constructed to facilitate the electrochemical oxidation or reduction of the analyte. The sensing layer may include, for example, a catalyst to catalyze a reaction of the analyte and produce a response at the working electrode, an electron transfer agent to transfer electrons between the analyte and the working electrode (or other component), or both.

A variety of different sensing layer configurations may be used. In certain embodiments, the sensing layer is deposited on the conductive material of a working electrode. The sensing layer may extend beyond the conductive material of the working electrode. In some cases, the sensing layer may also extend over other electrodes, e.g., over the counter electrode and/or reference electrode (or counter/reference is provided).

A sensing layer that is in direct contact with the working electrode may contain an electron transfer agent to transfer electrons directly or indirectly between the analyte and the working electrode, and/or a catalyst to facilitate a reaction of the analyte. For example, a glucose, lactate, or oxygen electrode may be formed having a sensing layer which contains a catalyst, including glucose oxidase, glucose dehydrogenase, lactate oxidase, or laccase, respectively, and an electron transfer agent that facilitates the electrooxidation of the glucose, lactate, or oxygen, respectively.

In other embodiments the sensing layer is not deposited directly on the working electrode. Instead, the sensing layer 508 may be spaced apart from the working electrode, and separated from the working electrode, e.g., by a separation layer. A separation layer may include one or more membranes or films or a physical distance. In addition to separating the working electrode from the sensing layer, the separation layer may also act as a mass transport limiting layer and/or an interferent eliminating layer and/or a biocompatible layer.

In certain embodiments which include more than one working electrode, one or more of the working electrodes may not have a corresponding sensing layer, or may have a sensing layer which does not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing layers by, for example, subtracting the signal.

In certain embodiments, the sensing layer includes one or more electron transfer agents.

Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes including ferrocene. Examples of inorganic redox species are hexacyanoferrate (III), ruthenium hexamine, etc. Additional examples include those described in U.S. Pat. Nos. 6,736,957, 7,501,053 and 7,754,093, the disclosures of each of which are incorporated herein by reference in their entirety.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

Embodiments of polymeric electron transfer agents may contain a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation. Another example of an ionically-bound mediator is a positively charged polymer including quaternized poly(4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, including 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole).

Embodiments may employ electron transfer agents having a redox potential ranging from about −200 mV to about +200 mV versus the standard calomel electrode (SCE). The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, including a glucose oxidase, glucose dehydrogenase (e.g., pyrroloquinoline quinone (PQQ), dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent, which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

In certain embodiments, the sensor operates at a low oxidizing potential, e.g., a potential of about +40 mV vs. Ag/AgCl. This sensing layer uses, for example, an osmium (Os)-based mediator constructed for low potential operation. Accordingly, in certain embodiments the sensing element is a redox active component that includes (1) osmium-based mediator molecules that include (bidente) ligands, and (2) glucose oxidase enzyme molecules. These two constituents are combined together in the sensing layer of the sensor.

A mass transport limiting layer (not shown), e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating functions, etc.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly(ethylene glycol), hydroxyl or polyhydroxyl modifiers, may be used to enhance the biocompatibility of the polymer or the resulting membrane.

A membrane may be formed in situ by applying an alcohol-buffer solution of a crosslinker and a modified polymer over an enzyme-containing sensing layer and allowing the solution to cure for about one to two days or other appropriate time period. The crosslinker-polymer solution may be applied to the sensing layer by placing a droplet or droplets of the membrane solution on the sensor, by dipping the sensor into the membrane solution, by spraying the membrane solution on the sensor, and the like. Generally, the thickness of the membrane is controlled by the concentration of the membrane solution, by the number of droplets of the membrane solution applied, by the number of times the sensor is dipped in the membrane solution, by the volume of membrane solution sprayed on the sensor, or by any combination of these factors. A membrane applied in this manner may have any combination of the following functions: (1) mass transport limitation, i.e., reduction of the flux of analyte that can reach the sensing layer, (2) biocompatibility enhancement, or (3) interferent reduction.

In some instances, the membrane may form one or more bonds with the sensing layer. By bonds is meant any type of an interaction between atoms or molecules that allows chemical compounds to form associations with each other, such as, but not limited to, covalent bonds, ionic bonds, dipole-dipole interactions, hydrogen bonds, London dispersion forces, and the like. For example, in situ polymerization of the membrane can form crosslinks between the polymers of the membrane and the polymers in the sensing layer. In certain embodiments, crosslinking of the membrane to the sensing layer facilitates a reduction in the occurrence of delamination of the membrane from the sensing layer.

In certain embodiments, the sensing system detects hydrogen peroxide to infer glucose levels. For example, a hydrogen peroxide-detecting sensor may be constructed in which a sensing layer includes enzyme such as glucose oxides, glucose dehydrogenase, or the like, and is positioned proximate to the working electrode. The sensing layer may be covered by one or more layers, e.g., a membrane that is selectively permeable to glucose. Once the glucose passes through the membrane, it is oxidized by the enzyme and reduced glucose oxidase can then be oxidized by reacting with molecular oxygen to produce hydrogen peroxide.

Certain embodiments include a hydrogen peroxide-detecting sensor constructed from a sensing layer prepared by combining together, for example: (1) a redox mediator having a transition metal complex including an Os polypyridyl complex with oxidation potentials of about +200 mV vs. SCE, and (2) periodate oxidized horseradish peroxidase (HRP). Such a sensor functions in a reductive mode; the working electrode is controlled at a potential negative to that of the Os complex, resulting in mediated reduction of hydrogen peroxide through the HRP catalyst.

In another example, a potentiometric sensor can be constructed as follows. A glucose-sensing layer is constructed by combining together (1) a redox mediator having a transition metal complex including Os polypyridyl complexes with oxidation potentials from about −200 mV to +200 mV vs. SCE, and (2) glucose oxidase. This sensor can then be used in a potentiometric mode, by exposing the sensor to a glucose containing solution, under conditions of zero current flow, and allowing the ratio of reduced/oxidized Os to reach an equilibrium value. The reduced/oxidized Os ratio varies in a reproducible way with the glucose concentration, and will cause the electrode's potential to vary in a similar way.

The substrate may be formed using a variety of non-conducting materials, including, for example, polymeric or plastic materials and ceramic materials. Suitable materials for a particular sensor may be determined, at least in part, based on the desired use of the sensor and properties of the materials.

In some embodiments, the substrate is flexible. For example, if the sensor is configured for implantation into a user, then the sensor may be made flexible (although rigid sensors may also be used for implantable sensors) to reduce pain to the user and damage to the tissue caused by the implantation of and/or the wearing of the sensor. A flexible substrate often increases the user's comfort and allows a wider range of activities. Suitable materials for a flexible substrate include, for example, non-conducting plastic or polymeric materials and other non-conducting, flexible, deformable materials. Examples of useful plastic or polymeric materials include thermoplastics such as polycarbonates, polyesters (e.g., Mylar™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate).

In other embodiments, the sensors are made using a relatively rigid substrate to, for example, provide structural support against bending or breaking. Examples of rigid materials that may be used as the substrate include poorly conducting ceramics, such as aluminum oxide and silicon dioxide. An implantable sensor having a rigid substrate may have a sharp point and/or a sharp edge to aid in implantation of a sensor without an additional insertion device.

It will be appreciated that for many sensors and sensor applications, both rigid and flexible sensors will operate adequately. The flexibility of the sensor may also be controlled and varied along a continuum by changing, for example, the composition and/or thickness of the substrate.

In addition to considerations regarding flexibility, it is often desirable that implantable sensors should have a substrate which is physiologically harmless, for example, a substrate approved by a regulatory agency or private institution for in vivo use.

The sensor may include optional features to facilitate insertion of an implantable sensor. For example, the sensor may be pointed at the tip to ease insertion. In addition, the sensor may include a barb which assists in anchoring the sensor within the tissue of the user during operation of the sensor. However, the barb is typically small enough so that little damage is caused to the subcutaneous tissue when the sensor is removed for replacement.

An implantable sensor may also, optionally, have an anticlotting agent disposed on a portion of the substrate which is implanted into a user. This anticlotting agent may reduce or eliminate the clotting of blood or other body fluid around the sensor, particularly after insertion of the sensor. Blood clots may foul the sensor or irreproducibly reduce the amount of analyte which diffuses into the sensor. Examples of useful anticlotting agents include heparin and tissue plasminogen activator (TPA), as well as other known anticlotting agents.

The anticlotting agent may be applied to at least a portion of that part of the sensor that is to be implanted. The anticlotting agent may be applied, for example, by bath, spraying, brushing, or dipping, etc. The anticlotting agent is allowed to dry on the sensor. The anticlotting agent may be immobilized on the surface of the sensor or it may be allowed to diffuse away from the sensor surface. The quantities of anticlotting agent disposed on the sensor may be below the amounts typically used for treatment of medical conditions involving blood clots and, therefore, have only a limited, localized effect.

### Insertion Device

An insertion device can be used to subcutaneously insert the sensor into the user. The insertion device is typically formed using structurally rigid materials, such as metal or rigid plastic. Materials may include stainless steel and ABS (acrylonitrile-butadiene-styrene) plastic. In some embodiments, the insertion device is pointed and/or sharp at the tip to facilitate penetration of the skin of the user. A sharp, thin insertion device may reduce pain felt by the user upon insertion of the sensor. In other embodiments, the tip of the insertion device has other shapes, including a blunt or flat shape. These embodiments may be useful when the insertion device does not penetrate the skin but rather serves as a structural support for the sensor as the sensor is pushed into the skin.

### Sensor Control Unit

The sensor control unit can be integrated in the sensor, part or all of which is subcutaneously implanted or it can be configured to be placed on the skin of a user. The sensor control unit is optionally formed in a shape that is comfortable to the user and which may permit concealment, for example, under a user's clothing. The thigh, leg, upper arm, shoulder, or abdomen are convenient parts of the user's body for placement of the sensor control unit to maintain concealment. However, the sensor control unit may be positioned on other portions of the user's body. One embodiment of the sensor control unit has a thin, oval shape to enhance concealment. However, other shapes and sizes may be used.

The particular profile, as well as the height, width, length, weight, and volume of the sensor control unit may vary and depends, at least in part, on the components and associated functions included in the sensor control unit. In general, the sensor control unit includes a housing typically formed as a single integral unit that rests on the skin of the user. The housing typically contains most or all of the electronic components of the sensor control unit.

The housing of the sensor control unit may be formed using a variety of materials, including, for example, plastic and polymeric materials, such as rigid thermoplastics and engineering thermoplastics. Suitable materials include, for example, polyvinyl chloride, polyethylene, polypropylene, polystyrene, ABS polymers, and copolymers thereof. The housing of the sensor control unit may be formed using a variety of techniques including, for example, injection molding, compression molding, casting, and other molding methods. Hollow or recessed regions may be formed in the housing of the sensor control unit. The electronic components of the sensor control unit and/or other items, including a battery or a speaker for an audible alarm, may be placed in the hollow or recessed areas.

The sensor control unit is typically attached to the skin of the user, for example, by adhering the sensor control unit directly to the skin of the user with an adhesive provided on at least a portion of the housing of the sensor control unit which contacts the skin or by suturing the sensor control unit to the skin through suture openings in the sensor control unit.

When positioned on the skin of a user, the sensor and the electronic components within the sensor control unit are coupled via conductive contacts. The one or more working electrodes, counter electrode (or counter/reference electrode), optional reference electrode, and optional temperature probe are attached to individual conductive contacts. For example, the conductive contacts are provided on the interior of the sensor control unit. Other embodiments of the sensor control unit have the conductive contacts disposed on the exterior of the housing. The placement of the conductive contacts is such that they are in contact with the contact pads on the sensor when the sensor is properly positioned within the sensor control unit.

### Sensor Control Unit Electronics

The sensor control unit also typically includes at least a portion of the electronic components that operate the sensor and the analyte monitoring device system. The electronic components of the sensor control unit typically include a power supply for operating the sensor control unit and the sensor, a sensor circuit for obtaining signals from and operating the sensor, a measurement circuit that converts sensor signals to a desired format, and a processing circuit that, at minimum, obtains signals from the sensor circuit and/or measurement circuit and provides the signals to an optional transmitter. In some embodiments, the processing circuit may also partially or completely evaluate the signals from the sensor and convey the resulting data to the optional transmitter and/or activate an optional alarm system if the analyte level exceeds a threshold. The processing circuit often includes digital logic circuitry.

The sensor control unit may optionally contain a transmitter for transmitting the sensor signals or processed data from the processing circuit to a receiver/display unit; a data storage unit for temporarily or permanently storing data from the processing circuit; a temperature probe circuit for receiving signals from and operating a temperature probe; a reference voltage generator for providing a reference voltage for comparison with sensor-generated signals; and/or a watchdog circuit that monitors the operation of the electronic components in the sensor control unit.

Moreover, the sensor control unit may also include digital and/or analog components utilizing semiconductor devices, including transistors. To operate these semiconductor devices, the sensor control unit may include other components including, for example, a bias control generator to correctly bias analog and digital semiconductor devices, an oscillator to provide a clock signal, and a digital logic and timing component to provide timing signals and logic operations for the digital components of the circuit.

As an example of the operation of these components, the sensor circuit and the optional temperature probe circuit provide raw signals from the sensor to the measurement circuit. The measurement circuit converts the raw signals to a desired format, using for example, a current-to-voltage converter, current-to-frequency converter, and/or a binary counter or other indicator that produces a signal proportional to the absolute value of the raw signal. This may be used, for example, to convert the raw signal to a format that can be used by digital logic circuits. The processing circuit may then, optionally, evaluate the data and provide commands to operate the electronics.

### Calibration

Sensors may be configured to require no system calibration or no user calibration. For example, a sensor may be factory calibrated and need not require further calibrating. In certain embodiments, calibration may be required, but may be done without user intervention, i.e., may be automatic. In those embodiments in which calibration by the user is required, the calibration may be according to a predetermined schedule or may be dynamic, i.e., the time for which may be determined by the system on a real-time basis according to various factors, including, but not limited to, glucose concentration and/or temperature and/or rate of change of glucose, etc.

In addition to a transmitter, an optional receiver may be included in the sensor control unit. In some cases, the transmitter is a transceiver, operating as both a transmitter and a receiver. The receiver may be used to receive calibration data for the sensor. The calibration data may be used by the processing circuit to correct signals from the sensor. This calibration data may be transmitted by the receiver/display unit or from some other source such as a control unit in a doctor's office. In addition, the optional receiver may be used to receive a signal from the receiver/display units to direct the transmitter, for example, to change frequencies or frequency bands, to activate or deactivate the optional alarm system and/or to direct the transmitter to transmit at a higher rate.

Calibration data may be obtained in a variety of ways. For instance, the calibration data may be factory-determined calibration measurements which can be input into the sensor control unit using the receiver or may alternatively be stored in a calibration data storage unit within the sensor control unit itself (in which case a receiver may not be needed). The calibration data storage unit may be, for example, a readable or readable/writeable memory circuit.

Calibration may be accomplished using an in vitro test strip (or other reference), e.g., a small sample test strip such as a test strip that requires less than about 1 microliter of sample (for example FreeStyle® blood glucose monitoring test strips from Abbott Diabetes Care, Alameda, Calif.). For example, test strips that require less than about 1 nanoliter of sample may be used. In certain embodiments, a sensor may be calibrated using only one sample of body fluid per calibration event. For example, a user need only lance a body part one time to obtain a sample for a calibration event (e.g., for a test strip), or may lance more than one time within a short period of time if an insufficient volume of sample is firstly obtained. Embodiments include obtaining and using multiple samples of body fluid for a given calibration event, where glucose values of each sample are substantially similar. Data obtained from a given calibration event may be used independently to calibrate or combined with data obtained from previous calibration events, e.g., averaged including weighted averaged, etc., to calibrate. In certain embodiments, a system need only be calibrated once by a user, where recalibration of the system is not required.

Alternative or additional calibration data may be provided based on tests performed by a health care professional or by the user. For example, it is common for diabetic individuals to determine their own blood glucose concentration using commercially available testing kits. The results of this test is input into the sensor control unit either directly, if an appropriate input device (e.g., a keypad, an optical signal receiver, or a port for connection to a keypad or computer) is incorporated in the sensor control unit, or indirectly by inputting the calibration data into the receiver/display unit and transmitting the calibration data to the sensor control unit.

Other methods of independently determining analyte levels may also be used to obtain calibration data. This type of calibration data may supplant or supplement factory-determined calibration values.

In some embodiments of the invention, calibration data may be required at periodic intervals, for example, every eight hours, once a day, or once a week, to confirm that accurate analyte levels are being reported. Calibration may also be required each time a new sensor is implanted or if the sensor exceeds a threshold minimum or maximum value or if the rate of change in the sensor signal exceeds a threshold value. In some cases, it may be necessary to wait a period of time after the implantation of the sensor before calibrating to allow the sensor to achieve equilibrium. In some embodiments, the sensor is calibrated only after it has been inserted. In other embodiments, no calibration of the sensor is needed.

### Analyte Monitoring Device

In some embodiments of the invention, the analyte monitoring device includes a sensor control unit and a sensor. In these embodiments, the processing circuit of the sensor control unit is able to determine a level of the analyte and activate an alarm system if the analyte level exceeds a threshold value. The sensor control unit, in these embodiments, has an alarm system and may also include a display, such as an LCD or LED display.

A threshold value is exceeded if the datapoint has a value that is beyond the threshold value in a direction indicating a particular condition. For example, a datapoint which correlates to a glucose level of 200 mg/dL exceeds a threshold value for hyperglycemia of 180 mg/dL, because the datapoint indicates that the user has entered a hyperglycemic state. As another example, a datapoint which correlates to a glucose level of 65 mg/dL exceeds a threshold value for hypoglycemia of 70 mg/dL because the datapoint indicates that the user is hypoglycemic as defined by the threshold value. However, a datapoint which correlates to a glucose level of 75 mg/dL would not exceed the same threshold value for hypoglycemia because the datapoint does not indicate that particular condition as defined by the chosen threshold value.

An alarm may also be activated if the sensor readings indicate a value that is outside of (e.g., above or below) a measurement range of the sensor. For glucose, the physiologically relevant measurement range is typically 30-400 mg/dL, including 40-300 mg/dL and 50-250 mg/dL, of glucose in the interstitial fluid.

The alarm system may also, or alternatively, be activated when the rate of change or acceleration of the rate of change in analyte level increase or decrease reaches or exceeds a threshold rate or acceleration. For example, in the case of a subcutaneous glucose monitor, the alarm system may be activated if the rate of change in glucose concentration exceeds a threshold value which may indicate that a hyperglycemic or hypoglycemic condition is likely to occur. In some cases, the alarm system is activated if the acceleration of the rate of change in glucose concentration exceeds a threshold value which may indicate that a hyperglycemic or hypoglycemic condition is likely to occur.

A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

### Drug Delivery System

The subject invention also includes sensors used in sensor-based drug delivery systems.

The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a processing unit such as a transmitter, a receiver/display unit, and a drug administration system. In some cases, some or all components may be integrated in a single unit. A sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or implanted insulin pump.

Each of the various references, presentations, publications, provisional and/or non-provisional U.S. patent applications, U.S. patents, non-U.S. patent applications, and/or non-U.S. patents that have been identified herein, is incorporated herein by reference in its entirety.

Other embodiments and modifications within the scope of the present disclosure will be apparent to those skilled in the relevant art. Various modifications, processes, as well as numerous structures to which the embodiments of the invention may be applicable will be readily apparent to those of skill in the art to which the invention is directed upon review of the specification. Various aspects and features of the invention may have been explained or described in relation to understandings, beliefs, theories, underlying assumptions, and/or working or prophetic examples, although it will be understood that the invention is not bound to any particular understanding, belief, theory, underlying assumption, and/or working or prophetic example. Although various aspects and features of the invention may have been described largely with respect to applications, or more specifically, medical applications, involving diabetic humans, it will be understood that such aspects and features also relate to any of a variety of applications involving non-diabetic humans and any and all other animals. Further, although various aspects and features of the invention may have been described largely with respect to applications involving partially implanted sensors, such as transcutaneous or subcutaneous sensors, it will be understood that such aspects and features also relate to any of a variety of sensors that are suitable for use in connection with the body of an animal or a human, such as those suitable for use as fully implanted in the body of an animal or a human. Finally, although the various aspects and features of the invention have been described with respect to various embodiments and specific examples herein, all of which may be made or carried out conventionally, it will be understood that the invention is entitled to protection within the full scope of the appended claims.

The following examples are put forth so as to provide those of ordinary skill in the art with a complete disclosure and description of how to make and use the embodiments of the invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Sensors Having Sensing Layers Incorporating a Thickener

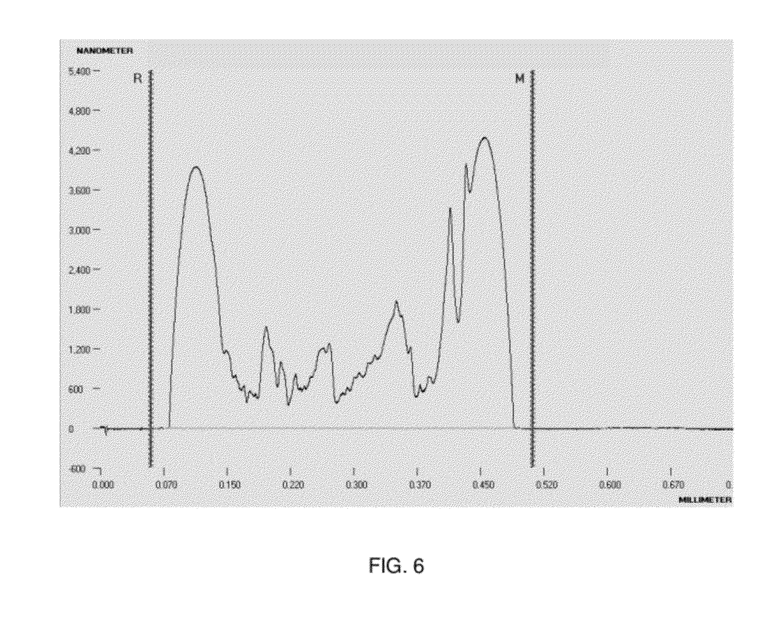

FIG. 6 illustrates problems that may be associated with sensor membrane fabrication. FIG. 6 shows a profilometer graph of a control spot (i.e., no thickener) of a sensing layer formulation. FIG. 6 shows the so-called “coffee ring” effect which results in poor uniformity and/or distribution of one or more components of the sensing layer of a sensor (e.g., an enzyme-containing sensing layer of such devices). In order to address these issues a thickener was incorporated into the sensing layer of the sensor.

Experiments were performed to test sensing layer formulations that included a thickener deposited on a gold substrate. The thickener used was a modified urea-based polymer thickener, BYK-420 (BYK Chemie GmbH, Wesel, Germany).

The sensing layer formulation was prepared as follows. Solutions of glucose oxidase, polymer bound redox mediator (see e.g., U.S. Pat. Nos. 6,605,200 and 6,605,201, the disclosures of which are incorporated herein by reverence in their entirety) and PEG 400 with 30 mg/mL concentration in 10 mM HEPES were prepared. 410 μL of glucose oxidase solution, 340 μL of polymer bound redox mediator solution and 250 μL of PEG 400 solution were mixed together to give 1 mL of 3% solids sensing layer solution. To the sensing layer solution was added 15 μL (1.5% (v/v)) or 30 μL (3% (v/v)) of BYK-420 solution (BYK Chemie GmbH, Wesel, Germany). The resulting sensing layer formulation was mixed for at least 1 hour before dispensing onto a gold substrate.

The membrane solution was prepared as follows. A heterocyclic nitrogen-containing polymer (see e.g., U.S. Pat. No. 6,932,894, the disclosure of which is incorporated herein by reverence in its entirety) was dissolved in 80% ethanol and 20% 10 mM HEPES solution to a concentration of 140 mg/mL. A solution of Gly3 was prepared at a concentration of 35 mg/mL in 80% ethanol and 20% 10 mM HEPES solution. To 4 mL of heterocyclic nitrogen-containing polymer solution was added 1 mL of Gly3 solution. The resulting membrane solution was stirred for 30 minutes before dipping.

For sensing layer formulations that included 1.5% BYK-420, about 12 nL of sensing layer formulation was deposited onto a gold substrate. Sensing layers deposited on the gold substrate were dipped in the membrane solution two times as a 55 mm/s exit speed. For sensing layer formulations that included 1.5% BYK-420, 80 sensors were prepared (40 filtered, and 40 unfiltered).

For sensing layer formulations that included 3% BYK-420, about 10 mL of sensing layer formulation was deposited onto a gold substrate. Sensing layers deposited on the gold substrate were dipped in the membrane solution two times as a 55 mm/s exit speed. For sensing layer formulations that included 3% BYK-420, 40 sensors were prepared.

In a comparison between a sensing layer formulation deposited on a gold substrate in which the sensing layer formulation was not supplemented with a thickener and a sensing layer formulation deposited on a gold substrate in which a sensing layer formulation included a thickener, the thickener-treated sensing layer is smoother, more homogenous and uniform in distribution as compared to the sensing layer lacking the thickener (see FIG. 6). For example, FIG. 7 shows a profilometer graph of a sensing layer that included 3% (v/v) of the thickener BYK-420.

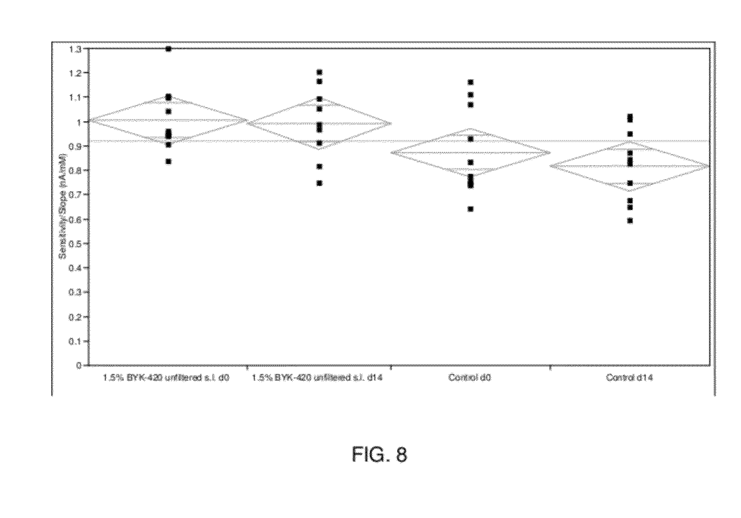

FIG. 8 shows graphs of one-way analysis of variance (ANOVA) of sensitivity/slope (nA/mM) for sensing layer formulations that included 1.5% BYK-420 at 0 days and at 14 days. As demonstrated in FIG. 8, the thickener supplemented sensing layer sensors had greater sensitivities than the control sensors that did not include a thickener. In addition, the thickener supplemented sensing layer sensors maintained 99% of their sensitivity after 14 days. One-way ANOVA data of sensitivity/slope (nA/mM) is shown in Table 1 below.

TABLE 1
One-way Analysis of Variance (ANOVA) of Sensitivity/Slope (nA/mM)
Summary of Fit

| | |
|---|---|
| Rsquare | 0.228845 |
| Adjusted Rsquare | 0.162746 |
| Root Mean Square Error | 0.154981 |
| Mean of Response | 0.921769 |
| Observations (or Sum Wgts) | 39 |

Analysis of Variance

| Source | DF | Sum of Squares | Mean Square | F Ratio | Prob > F |
|---|---|---|---|---|---|
| Lot | 3 | 0.2494742 | 0.083158 | 3.4622 | 0.0265 |
| Error | 35 | 0.8406687 | 0.024019 | | |
| C. Total | 38 | 1.0901429 | | | |

Means for One-Way ANOVA

| Level | Number | Mean | Std Error | Lower 95% | Upper 95% |
|---|---|---|---|---|---|
| 1.5% BYK- | 10 | 1.00750 | 0.04901 | 0.90801 | 1.1070 |
| 420, unfiltered | | | | | |
| sensing layer, | | | | | |
| 0 days | | | | | |
| 1.5% BYK- | 9 | 0.99356 | 0.05166 | 0.88868 | 1.0984 |
| 420, unfiltered | | | | | |
| sensing layer, | | | | | |
| 14 days | | | | | |
| control, 0 days | 10 | 0.87540 | 0.04901 | 0.77591 | 0.9749 |
| control, 14 days | 10 | 0.81780 | 0.04901 | 0.71831 | 0.9173 |

Standard error used a pooled estimate of error variance.

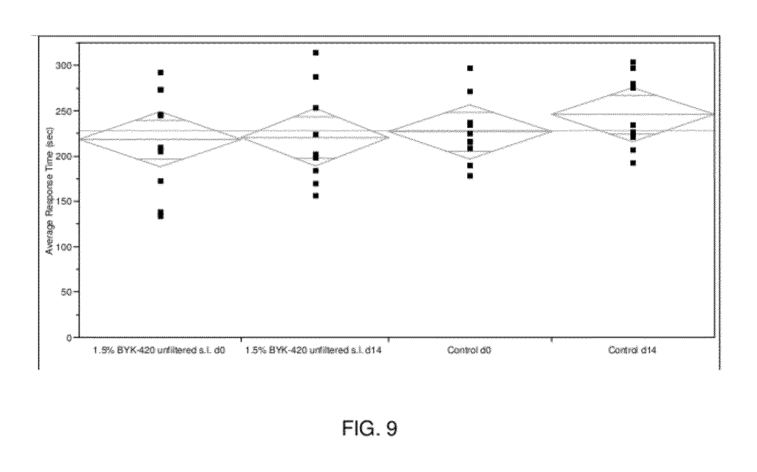

FIG. 9 shows graphs of one-way analysis of variance (ANOVA) of average response time (sec) for sensing layer formulations that included 1.5% BYK-420 at 0 days and at 14 days. As demonstrated in FIG. 9, the thickener supplemented sensing layer sensors had lower average response times than the control sensors that did not include a thickener. In addition, the thickener supplemented sensing layer sensors maintained their lower average response times after 14 days as compared to the control sensors. One-way ANOVA data of average response times (sec) is shown in Table 2 below.

TABLE 2
One-way Analysis of Variance (ANOVA) of Average Response Time (sec)
Summary of Fit

| | |
|---|---|
| Rsquare | 0.05583 |
| Adjusted Rsquare | −0.0251 |
| Root Mean Square Error | 47.06387 |
| Mean of Response | 228.4846 |
| Observations (or Sum Wgts) | 39 |

Analysis of Variance

| Source | DF | Sum of Squares | Mean Square | F Ratio | Prob > F |
|---|---|---|---|---|---|
| Lot | 3 | 4584.137 | 1528.05 | 0.6899 | .05644 |
| Error | 35 | 77525.264 | 2215.01 | | |
| C. Total | 38 | 82109.401 | | | |

Means for One-Way ANOVA

| Level | Number | Mean | Std Error | Lower 95% | Upper 95% |
|---|---|---|---|---|---|
| 1.5% BYK- | 10 | 218.770 | 14.883 | 188.56 | 248.98 |
| 420, unfiltered | | | | | |
| sensing layer, | | | | | |
| 0 days | | | | | |
| 1.5% BYK- | 9 | 221.078 | 15.688 | 189.23 | 252.93 |
| 420, unfiltered | | | | | |
| sensing layer, | | | | | |
| 14 days | | | | | |
| control, 0 days | 10 | 227.175 | 14.883 | 196.96 | 257.39 |
| control, 14 days | 10 | 246.175 | 14.883 | 215.96 | 276.39 |

Standard error used a pooled estimate of error variance.

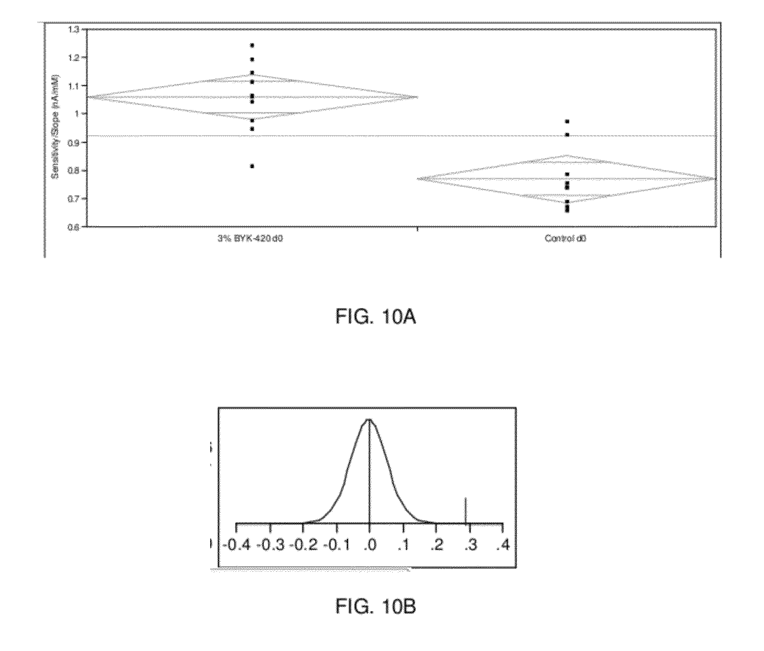

FIG. 10A shows graphs of one-way analysis of variance (ANOVA) of sensitivity/slope (nA/mM) for sensing layer formulations that included 3% BYK-420 at 0 days. As demonstrated in FIG. 10A, the thickener-supplemented sensing layer sensors had greater sensitivities than the control sensors that did not include a thickener. FIG. 10B shows a t-test graph for the data from experiments shown in FIG. 10A. One-way ANOVA data of sensitivity/slope (nA/mM) is shown in Table 3 below.

TABLE 3
One-way Analysis of Variance (ANOVA) of Sensitivity/Slope (nA/mM)
Summary of Fit

| | |
|---|---|
| Rsquare | 0.624048 |
| Adjusted Rsquare | 0.601934 |
| Root Mean Square Error | 0.118916 |
| Mean of Response | 0.923316 |
| Observations (or Sum Wgts) | 19 |

t Test
3% BYK-420, 0 days - Control, 0 days
Assuming equal variances

| | | | |
|---|---|---|---|
| Difference | 0.290244 | t Ratio | 5.312116 |
| Std Err Dif | 0.054638 | DF | 17 |
| Upper CL Dif | 0.405521 | Prob > \|t\| | <0.0001 |
| Lower CL Dif | 0.174968 | Prob > t | <0.0001 |
| Confidence | 0.95 | Prob < t | 1.0000 |

Analysis of Variance

| Source | DF | Sum of Squares | Mean Square | F Ratio | Prob > F |
|---|---|---|---|---|---|
| Lot | 1 | 0.39904028 | 0.399040 | 28.2186 | <0.0001 |
| Error | 17 | 0.24039782 | 0.14141 | | |
| C. Total | 18 | 0.63943811 | | | |

Means for One-Way ANOVA

| Level | Number | Mean | Std Error | Lower 95% | Upper 95% |
|---|---|---|---|---|---|
| 3% BYK-420, | 10 | 1.06080 | 0.03760 | 0.98146 | 1.1401 |
| 0 days | | | | | |
| control, 0 days | 9 | 0.77056 | 0.03964 | 0.68693 | 0.8542 |

Standard error used a pooled estimate of error variance.

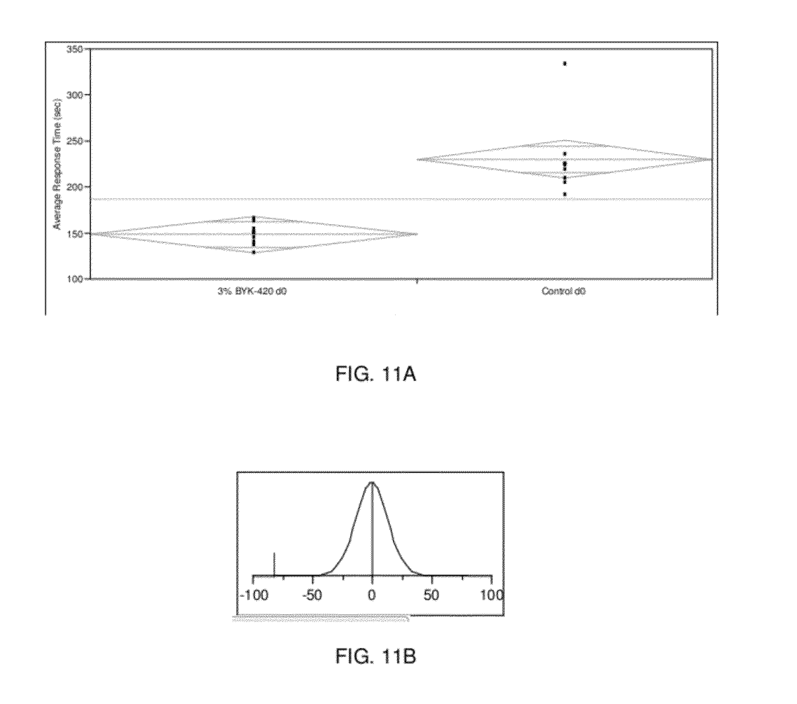

FIG. 11A shows graphs of one-way analysis of variance (ANOVA) of average response time (sec) for sensing layer formulations that included 3% BYK-420 at 0 days. As demonstrated in FIG. 11A, the thickener-supplemented sensing layer sensors had lower average response times than the control sensors that did not include a thickener. FIG. 11B shows a t-test graph for the data from experiments shown in FIG. 11A. One-way ANOVA data of sensitivity/slope (nA/mM) is shown in Table 4 below.

TABLE 4
One-way Analysis of Variance (ANOVA) of Average Response Time (sec)
Summary of Fit

| | |
|---|---|
| Rsquare | 0.683501 |
| Adjusted Rsquare | 0.664883 |
| Root Mean Square Error | 29.25985 |
| Mean of Response | 187.5605 |
| Observations (or Sum Wgts) | 19 |

t Test
3% BYK-420, 0 days - Control, 0 days
Assuming equal variances

| | | | |
|---|---|---|---|
| Difference | −81.46 | t Ratio | −6.0591 |
| Std Err Dif | 13.44 | DF | 17 |
| Upper CL Dif | −53.09 | Prob > \|t\| | <0.0001 |
| Lower CL Dif | −109.82 | Prob > t | 1.0000 |
| Confidence | 0.95 | Prob < t | <0.0001 |

Analysis of Variance

| Source | DF | Sum of Squares | Mean Square | F Ratio | Prob > F |
|---|---|---|---|---|---|
| Lot | 1 | 31431.127 | 31431.1 | 36.7126 | <0.0001 |
| Error | 17 | 14554.361 | 856.1 | | |
| C. Total | 18 | 45985.488 | | | |

Means for One-Way ANOVA

| Level | Number | Mean | Std Error | Lower 95% | Upper 95% |
|---|---|---|---|---|---|
| 3% BYK-420, | 10 | 148.975 | 9.2528 | 129.45 | 168.50 |
| 0 days | | | | | |
| control, 0 days | 9 | 230.433 | 9.7533 | 209.86 | 251.01 |

Standard error used a pooled estimate of error variance.

In conclusion, the experiments above show that the addition of a thickener to an analyte sensor formulation, such as a sensing layer, promoted the uniformity and/or distribution of the sensing layer formulation and substantial elimination of the “coffee ring” effect of settling of sensing layer components at the perimeter of the formulation on the sensor surface.

### Example 2

### Sensors Having Sensing Layers Incorporating an Enzyme Stabilizer

Experiments were performed to test sensing layer formulations that included an enzyme stabilizer. The enzyme stabilizer used was glutathione.

The sensing layer formulation was prepared as follows. Solutions of glucose oxidase, polymer bound redox mediator and PEG 400 with 30 mg/mL concentration in 10 mM HEPES were prepared. 410 μL of glucose oxidase solution, 340 μL of polymer bound redox mediator solution and 250 μL of PEG 400 solution were mixed together to give 1 mL of 3% solids sensing layer solution. To the sensing layer solution was added glutathione (5 mg to 20 mg). The resulting sensing layer formulation was mixed for at least 1 hour before dispensing onto a gold substrate. About 11 nL of sensing layer formulation was deposited onto a gold substrate. Sensors were prepared that had 0.25% glutathione (5 mg glutathione per 1 mL of 3% solids sensing layer solution), and 0.5% glutathione (10 mg glutathione per 1 mL of 3% solids sensing layer solution).

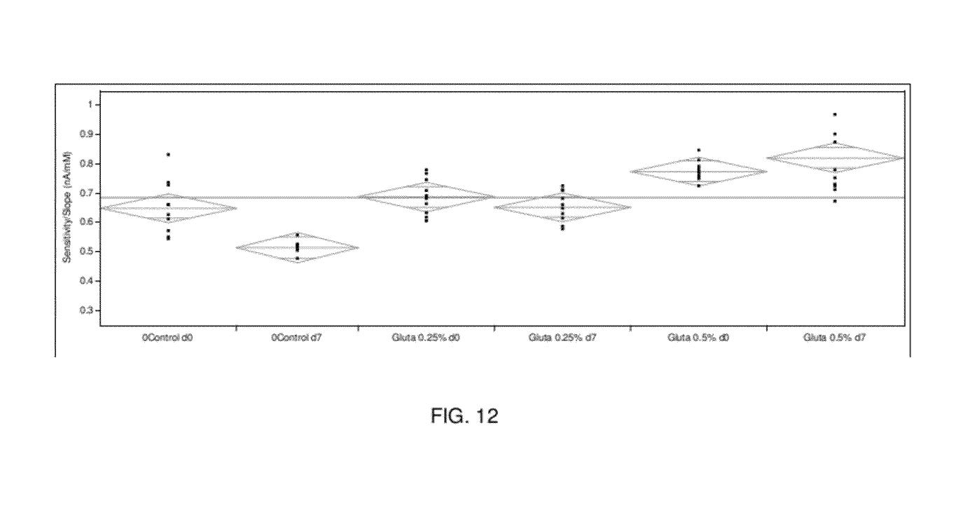

In a comparison between a sensing layer formulation deposited on a gold substrate in which the sensing layer formulation was not supplemented with an enzyme stabilizer and a sensing layer formulation deposited on a gold substrate in which a sensing layer formulation included an enzyme stabilizer, the enzyme stabilizer-treated sensing layer had a higher mean sensitivity/slope (nA/mM) than control sensors (see FIG. 12).

FIG. 12 shows graphs of one-way analysis of variance (ANOVA) of sensitivity/slope (nA/mM) for sensing layer formulations that included 0.25% and 0.5% glutathione at 0 days and at 7 days. As demonstrated in FIG. 12, the enzyme stabilizer supplemented sensing layer sensors had greater sensitivities than the control sensors that did not include an enzyme stabilizer. The sensing layer sensors supplemented with 0.25% glutathione enzyme stabilizer maintained 95% of their sensitivity after 7 days. The sensing layer sensors supplemented with 0.5% glutathione enzyme stabilizer had a mean sensitivity 106% of the starting mean sensitivity after 7 days. One-way ANOVA data of sensitivity/slope (nA/mM) is shown in Table 5 below.

TABLE 5
One-way Analysis of Variance (ANOVA) of Sensitivity/Slope (nA/mM)
Means for One-Way ANOVA

| Level | Number | Mean | Std Error | Lower 95% | Upper 95% |
|---|---|---|---|---|---|
| control, | 10 | 0.649400 | 0.02470 | 0.59985 | 0.69895 |
| 0 days | | | | | |
| control, | 9 | 0.513889 | 0.02604 | 0.46166 | 0.56612 |
| 7 days | | | | | |
| 0.25% | 10 | 0.678100 | 0.02470 | 0.63755 | 0.73665 |
| glutathione, | | | | | |
| 0 days | | | | | |
| 0.25% | 10 | 0.652400 | 0.02470 | 0.60285 | 0.70195 |
| glutathione, | | | | | |
| 7 days | | | | | |
| 0.5% | 10 | 0.774200 | 0.02470 | 0.72465 | 0.82375 |
| glutathione, | | | | | |
| 0 days | | | | | |
| 0.5% | 10 | 0.820700 | 0.02470 | 0.77115 | 0.87025 |
| glutathione, | | | | | |
| 7 days | | | | | |

Standard error used a pooled estimate of error variance.

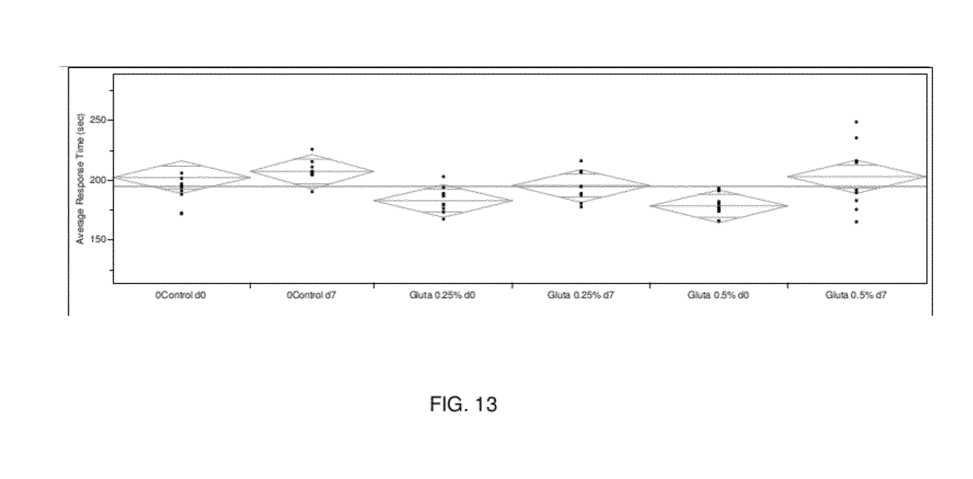

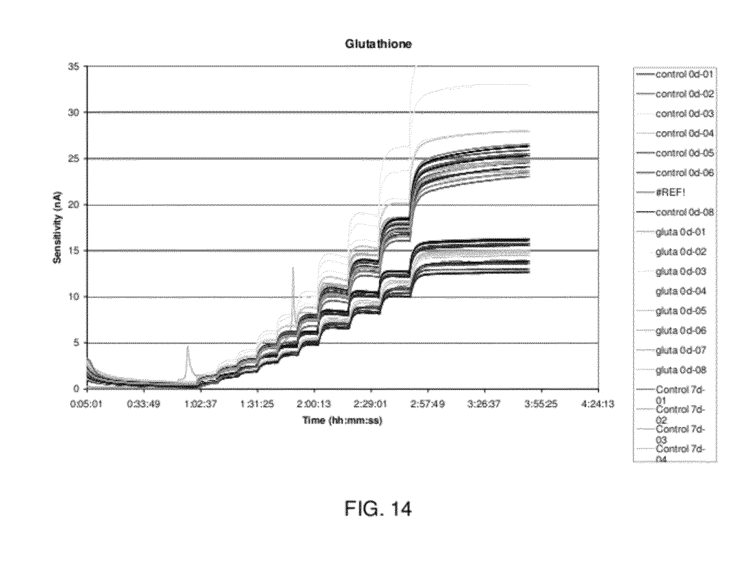

FIG. 13 shows graphs of one-way analysis of variance (ANOVA) of average response time (sec) for sensing layer formulations that included 0.25% and 0.5% glutathione at 0 days and at 7 days. As demonstrated in FIG. 12, the enzyme stabilizer supplemented sensing layer sensors had lower average response times than the control sensors that did not include an enzyme stabilizer. In addition, the enzyme stabilizer supplemented sensing layer sensors maintained their lower average response times after 7 days as compared to the control sensors. One-way ANOVA data of average response times (sec) is shown in Table 6 below. FIG. 14 shows graphs of sensitivity (nA) vs. time for sensors having sensing layer formulations that included glutathione. In certain embodiments, sensors having sensing layer formulations that included an enzyme stabilizer, such as glutathione, have a sensitivity (nA) that is 1.2 times, such as 1.4 times, or 1.6 times, or 1.8 times, or 2 times, or 2.2 times, or 2.4 times, or 2.6 times, or 2.8 times, or 3 times greater than sensors having sensing layer formulations lacking an enzyme stabilizer.

TABLE 6
One-way Analysis of Variance (ANOVA) of Average
Response Time (sec)
Means for One-Way ANOVA

| Level | Number | Mean | Std Error | Lower 95% | Upper 95% |
|---|---|---|---|---|---|
| control, | 10 | 202.400 | 6.8478 | 188.66 | 216.14 |
| 0 days | | | | | |
| control, | 9 | 207.389 | 7.2183 | 192.91 | 221.87 |
| 7 days | | | | | |
| 0.25% | 10 | 182.745 | 6.8478 | 169.01 | 196.48 |
| glutathione, | | | | | |
| 0 days | | | | | |
| 0.25% | 10 | 195.440 | 6.8478 | 181.70 | 209.18 |
| glutathione, | | | | | |
| 7 days | | | | | |
| 0.5% | 10 | 178.270 | 6.8478 | 164.53 | 192.01 |
| glutathione, | | | | | |
| 0 days | | | | | |
| 0.5% | 10 | 203.025 | 6.8478 | 189.29 | 216.76 |
| glutathione, | | | | | |
| 7 days | | | | | |

Standard error used a pooled estimate of error variance.

In conclusion, the experiments above show that the addition of an enzyme stabilizer to an analyte sensor formulation, such as a sensing layer, improved sensitivity and reduced sensitivity variation of the sensor.

### Example 3

Unless indicated otherwise, all of the chemical reagents are available from Aldrich Chemical Co. (Milwaukee, Wis.) or other sources. Methodologies and synthesis of monodentate, bidentate, terdentate and tetradentate ligands and methods for chemically bonding transition metal complexes to a polymer are described in detail in U.S. Pat. Nos. 6,605,200 and 6,605,201, the disclosures of which are herein incorporated by reference in their entirety. Below is an example of the synthesis of a pyridine boronic acid ligands as well as osmium complexes having at least one pyridine boronic acid ligand.

### Synthesis of a Pyridine Boronic Acid-bis-2-(2-Pyridyl)methylimidazole Osmium Complex

### Synthesis of 2-(2-pyridyl)imidazole

A solution of pyridine-2-carboxaldehyde (151.4 g, 1.41 moles) and glyoxal (40% in H₂O, 205 mL, 1.79 moles) in 300 mL of ethanol (EtOH) in a three-necked 1 L round-bottom flask fitted with a thermometer and an addition funnel was stirred in an ice bath. When the solution was cooled to below 5° C., concentrated NH₄OH (28-30%, 482 mL, 3.93 moles) was added dropwise through the addition funnel. The rate of the addition was controlled so that the temperature of the solution was maintained at below 5° C. After the addition, the stifling of the solution was continued in the ice bath for one hour and then at room temperature overnight. During the stifling process, the solution changed from light yellow to dark brown.

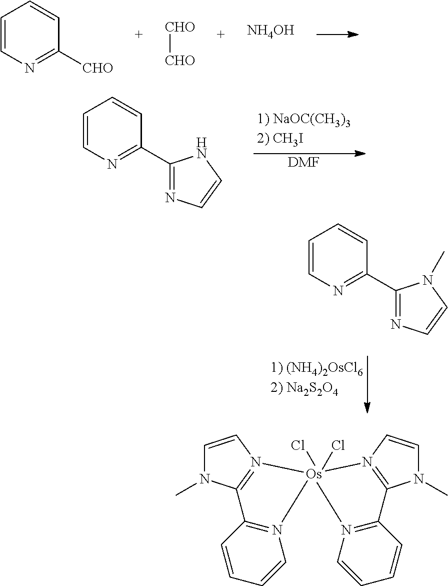

The solution was transferred to a 2 L round bottom flask and the EtOH solvent was removed by rotary evaporation. The resulting dark viscous material was transferred to a 4 L beaker with 700 mL of EtOAc 500 mL of saturated NaCl was added and the mixture was stirred for 2 hours. The solution was poured into a 2 L separation funnel and a dark tarry material was discarded. The organic layer was separated from the solution and the aqueous layer was extracted several times with EtOAc (500 mL EtOAc per extraction). The organic layer was then dried with anhydrous Na₂SO₄ overnight, whereupon the resulting mixture was gravity filtered, the Na₂SO₄ was washed with EtOAc (4×50 mL), and the solution was concentrated to about 300-400 mL by rotary evaporation. The concentrated solution was transferred to a 1 L Erlenmeyer flask and the volume was adjusted with more EtOAc to about 400-500 mL, as necessary. The solution stood at 4° C. for 1-2 days to form large amber crystals. The crystals were collected by suction filtration and washed with cold EtOAc (20-30 mL). The filtrate contained a large amount of product, so further concentration and crystallization procedures were performed. The crystals were combined and dried at 40-45° C. under high vacuum for 2 days. The yield of 2-(2-pyridyl)imidazole was about 75 g.

### Synthesis of 1-methyl-2-(2-pyridyl)imidazole

Pyridine-2-carboxaldehyde (50.5 g, 0.47 moles) and glyoxal (40% in H₂O, 68.3 mL, 0.60 moles) in 100-150 mL of ethanol (EtOH) in a three-necked 1 L round-bottom flask fitted with a thermometer and an addition funnel were stirred in an ice bath. When the solution was cooled to below 5° C., concentrated NH₄OH (28-30%, 161 mL, 1.31 moles) was added dropwise through the addition funnel. The rate of the addition was controlled so that the temperature of the solution was maintained at below 5° C. After the addition, the stifling of the solution was continued in the ice bath for one hour and then at room temperature overnight. During the stifling process, the solution changed from light yellow to dark brown.

The solution was transferred to a 1 L round bottom flask and the EtOH and H₂O solvent was removed by rotary evaporation at 50° C. The resulting material was dried further at about 50° C. under high vacuum for 24 hours and then dissolved in anhydrous dimethyl formamide (DMF), whereupon the solution was transferred with further DMF (total DMF 450-500 mL) to a three-necked 1 L round bottom flask equipped with a reflux condenser, and then stifled. Sodium t-butoxide (48.9 g, 0.51 moles) was added quickly via a funnel to obtain, with continued stifling for about 1 hour, a dark brown homogeneous solution. Methyl iodide (34.5 mL, 0.56 moles) was then added dropwise via an addition funnel over 1.5-2 hours, resulting in a white precipitate of NaI. The mixture was stirred at room temperature overnight, its color changing from dark brown to light brown. The mixture was then poured into a beaker containing 1.5 mL of EtOAc and suction-filtered using a Buchner funnel to remove the NaI precipitate. The precipitate was washed with additional EtOAc (3×100 mL). The filtrate was transferred to a 2 L round bottom flash and rotary evaporated to remove the EtOAc.

The resulting viscous material was transferred to a 1 L beaker with a minimum amount of EtOAc, which was then removed by rotary evaporation. The remaining DMF was removed by vacuum distillation using a low vacuum diaphragm pump and an oil bath. Upon complete removal of the DMF, the product was distilled at 100-110° C. under high vacuum. The yield of 1-methyl-2-(2-pyridyl)imidazole was about 36 g.

### Synthesis of Os(Py-MIM)₂Cl₂

1-methyl-2-(2-pyridyl)imidazole (3.4 g, 21.4 mmoles) and ammonium hexachloroosmiate (IV) (4.7 g, 10.7 mmoles) were combined with anhydrous ethylene glycol (86 mL) in a three-necked 250 mL round bottom flask, fitted with a reflux condenser, immersed in a temperature-controlled oil bath. The reaction mixture was degassed with N₂ for about 15 minutes. The mixture was stirred under N₂ while the heater was turned on to heat the oil bath, and the reaction proceeded at 130° C. for 2 hours and subsequently at 140° C. for about 28 hours until an intermediate that was formed in the reaction was completely converted to the final product. The solution was cooled to room temperature and then suction-filtered through a fritted funnel into a three-necked 250 mL round bottom flask, whereupon a small amount of orange precipitate left in the funnel was discarded. The solution (solution A) was then degassed with N₂ for 15 minutes and kept under N₂.

Deionized H₂O (320 mL) was then degassed with N₂ in a three-necked 500 mL round bottom flask cooled in an ice/water bath and equipped with a thermometer. After 15 minutes of degassing, sodium hydrosulfite (85%, 9.31 g, 53.5 mmoles) under N₂ was added immediately and degassing continued for another 10-15 minutes. The temperature of the solution (solution B) was below 5° C. Solution A was then added via a canula to solution B under rapid stifling for about 0.5 hour to form a fine dark purple precipitate of Os(Py-MIM)₂Cl₂. Stirring continued under N₂ for another 0.5 hour. The resulting suspension was suction-filtered through a 0.4 or 0.3 micron Nylon membrane. The suspension was transferred to the suction funnel via a canula under nitrogen to minimize air exposure. The dark purple precipitate was then washed with a minimum of ice cold water (2×5 mL). The precipitate was immediately dried by lyophilization for at least 24 hours. The yield of Os(Py-MIM)₂Cl₂ was about 5.6 g.

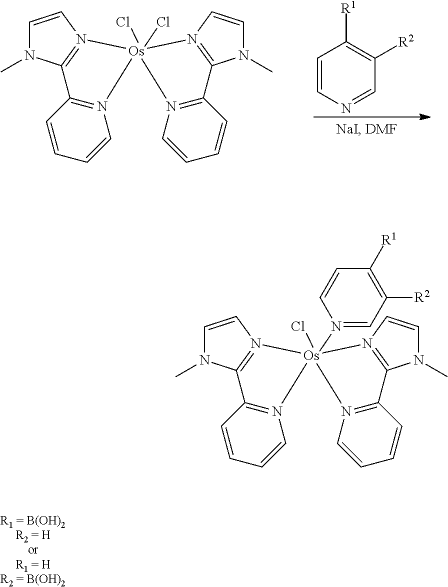

### Synthesis of [Os(Py-MIM)₂(MIM)Cl]²⁺2Cl⁻

Anhydrous ethanol (1 L) in a 2-L three-necked round bottom flask fitted with a reflux condenser was degassed with N₂ for 15 minutes. Os(Py-MIM)₂Cl₂ (3.1 g, 5.35 mmoles) was added quickly under N₂ via a funnel. The suspension was stirred and heated to reflux. 1-methylimidazole (0.43 mL, 5.35 mmoles) was then added at once via a syringe. Reflux continued until the reaction was completed. During the reaction, the solution changed from dark brown to purple-brown. The solution was cooled to room temperature and then suction-filtered through a fritted funnel. The solvent was then removed by rotary evaporation to give the crude product in its reduced form.

The product was transferred with 30-50 mL H₂O to a 400 mL beaker containing about 40 mL AG1×4 chloride resin from Bio-Rad, or preferably, 80 mL Dowex-1-chloride from Aldrich. The mixture was stirred in open air for about 24 hours to convert Os(II) to Os(III). The mixture was then suction-filtered and the resin was washed with H₂O (5×3 mL). The combined filtrate was concentrated to about 50 mL by rotary evaporation at 35° C. under vacuum.

The solution was loaded onto a LH-20 column (2″×22″), which was eluted with H.sub.2 O. 50 mL fractions were collected and analyzed by CV to find the major purple-brown band associated with the product. Fractions containing pure product were collected and concentrated by rotary evaporation to about 150 mL. The solution was then freeze-dried to give the product. The yield of [Os(Py-MIM)₂(MIM)Cl]²⁺2Cl⁻ was about 2.4 g.

### Synthesis of [Os(Py-MIM)₂(Py-B(OH)₂)Cl]²⁺2Cl⁻

To Os(Py-MIM)₂Cl₂ (100 mg, 0.17 mmol) in dimethylformamide (3.5 mL) under argon, is added sodium iodide (129 mg, 0.86 mmol) followed by 3- or 4-pyridineboronic acid (43 mg, 0.35 mmol). The mixture is stirred in the dark at 90° C. for 18 h, then oxidized under air for another 18 h in the presence of chloride resin, then filtered to yield 170-180 mg of the crude osmium pyridyl boronic acid (Cyclic voltammetry gives the following potentials vs. SCE in PBS buffer: −97 mV for 3-PBA and −80 mV for 4-BPA).

In summary, transition metal complexes described herein are particularly useful as redox mediators in electrochemical sensing applications. The redox mediators exchange electrons rapidly with analytes and working electrodes, are stable, are readily synthesized, and have redox potentials that are tailored for the electrooxidation of a variety of analytes, such as those in various biological fluids within the human body. If the redox potential of the enzyme used in a particular analyte-sensing application is negative relative to the redox potential of the mediator, the mediator is suitable for that analyte-sensing application. The advantageous properties and characteristics of the transition metal complexes make them useful for the electrochemical sensing of glucose, an application of particular importance in the diagnosis and monitoring of diabetes in human populations.

The present description should not be considered limited to the particular examples described above, but rather should be understood to cover all aspects as fairly set out in the attached claims. Various modifications, equivalent processes, as well as numerous structures to which embodiments of the present disclosure may be applicable will be readily apparent to those of skill in the art upon review of the instant specification.

1. An analyte sensor comprising:
a working electrode;
a counter electrode; and
a sensing layer disposed on the working electrode, wherein the sensing layer comprises an analyte-responsive enzyme, a redox mediator and a thickener comprising one or more of urea, a urea derivative, urethane, a urethane derivative, a polyvinyl pyrrolidone polymer and a polyvinyl pyrrolidone polymer derivative.

2. The analyte sensor of claim 1, wherein at least a portion of the analyte sensor is adapted to be subcutaneously positioned in a subject.

3. The analyte sensor of claim 1, wherein the sensing layer has an arcuate profile as measured using a profilometer.

4. The analyte sensor of claim 1, wherein the analyte sensor has a sensitivity that is 90% or more of its initial sensitivity after 14 days or more.

5. The analyte sensor of claim 1, wherein the analyte sensor has an average response time that is 35% or more lower than the average response time of a sensor that does not include a thickener.

6. The analyte sensor of claim 1, wherein the analyte-responsive enzyme and the redox mediator are distributed throughout the sensing layer.

7. The analyte sensor of claim 1, further comprising a membrane disposed over the sensing layer, wherein the membrane limits flux of analyte to the sensing layer.

8. The analyte sensor of claim 7, wherein only the sensing layer comprises the thickener.

9. The analyte sensor of claim 1, wherein the analyte sensor is a glucose sensor.

10. The analyte sensor of claim 9, wherein the analyte-responsive enzyme comprises a glucose-responsive enzyme.

11. The analyte sensor of claim 10, wherein the glucose-responsive enzyme comprises glucose oxidase.

12. The analyte sensor of claim 9, wherein the redox mediator comprises a ruthenium-containing complex or an osmium-containing complex.

13. The analyte sensor of claim 1, wherein the analyte sensor is an in vivo sensor.

14. The analyte sensor of claim 1, wherein the analyte sensor is an in vitro sensor.

15. A method for monitoring a level of an analyte in a subject, the method comprising:
positioning at least a portion of an analyte sensor into skin of a subject, wherein the analyte sensor comprises:
a working electrode;
a counter electrode; and
a sensing layer disposed on the working electrode, wherein the sensing layer comprises an analyte-responsive enzyme, a redox mediator and a thickener comprising one or more of urea, a urea derivative, urethane, a urethane derivative, a polyvinyl pyrrolidone polymer and a polyvinyl pyrrolidone polymer derivative, and
determining a level of an analyte over a period of time from signals generated by the analyte sensor,
wherein the determining over a period of time provides for monitoring the level of the analyte in the subject.

16. The method of claim 15, wherein the sensing layer has an arcuate profile as measured using a profilometer.

17. The method of claim 15, wherein the analyte sensor has a sensitivity that is 90% or more of its initial sensitivity after 14 days or more.

18. The method of claim 15, wherein the analyte sensor has an average response time that is 35% or more lower than the average response time of a sensor that does not include a thickener.

19. The method of claim 15, wherein the analyte-responsive enzyme and the redox mediator are distributed throughout the sensing layer.

20. The method of claim 15, wherein the analyte sensor further comprises a membrane disposed over the sensing layer, wherein the membrane limits flux of the analyte to the sensing layer.

21. The method of claim 20, wherein only the sensing layer comprises the thickener.

22. The method of claim 15, wherein the analyte sensor is a glucose sensor.

23. The method of claim 22, wherein the analyte-responsive enzyme comprises a glucose-responsive enzyme.

24. The method of claim 23, wherein the glucose-responsive enzyme comprises glucose oxidase.

25. The method of claim 22, wherein the redox mediator comprises a ruthenium-containing complex or an osmium-containing complex.

26. A method for monitoring a level of an analyte using an analyte monitoring system, the method comprising:
inserting at least a portion of an analyte sensor into skin of a patient, the analyte sensor comprising:
a working electrode;
a counter electrode; and
a sensing layer disposed on the working electrode, wherein the sensing layer comprises an analyte-responsive enzyme, a redox mediator and a thickener comprising one or more of urea, a urea derivative, urethane, a urethane derivative, a polyvinyl pyrrolidone polymer and a polyvinyl pyrrolidone polymer derivative;
attaching an analyte sensor control unit to the skin of the patient;
coupling a plurality of conductive contacts of the analyte sensor control unit to a plurality of contact pads of the analyte sensor;
collecting data, using the analyte sensor control unit, regarding a level of an analyte from signals generated by the analyte sensor; and
transmitting the collected data from the analyte sensor control unit to a receiver unit.

27. The method of claim 26, wherein the sensing layer has an arcuate profile as measured using a profilometer.

28. The method of claim 26, wherein the analyte sensor has a sensitivity that is 90% or more of its initial sensitivity after 14 days or more.

29. The method of claim 26, wherein the analyte sensor has an average response time that is 35% or more lower than the average response time of a sensor that does not include a thickener.

30. The method of claim 26, wherein the analyte is glucose.

31. The method of claim 26, wherein the collecting data comprises generating signals from the analyte sensor and processing the signals into data.

32. The method of claim 26, wherein the data comprise the signals from the analyte sensor.

33. The method of claim 26, further comprising activating an alarm if the data indicate an alarm condition.

34. The method of claim 26, further comprising administering a drug in response to the data.

35. The method of claim 34, wherein the drug is insulin.

36. The method of claim 26, further comprising obtaining a calibration value from a calibration device to calibrate the data.

37. The method of claim 36, wherein the calibration device is coupled to a display unit.

38. The method of claim 37, further comprising transmitting the calibration value from a transmitter in the display unit to a receiver in the analyte sensor control unit.

39. A method of fabricating an electrode for use in an analyte sensor, the method comprising:
contacting an electrode with a sensing layer, wherein the sensing layer comprises an analyte-responsive enzyme, a redox mediator and a thickener comprising one or more of urea, a urea derivative, urethane, a urethane derivative, a polyvinyl pyrrolidone polymer and a polyvinyl pyrrolidone polymer derivative.

40. The method of claim 39, wherein the sensing layer has an arcuate profile as measured using a profilometer.

41. The method of claim 39, wherein the analyte sensor has a sensitivity that is 90% or more of its initial sensitivity after 14 days or more.

42. The method of claim 39, wherein the analyte sensor has an average response time that is 35% or more lower than the average response time of a sensor that does not include a thickener.

43. The method of claim 39, wherein the analyte-responsive enzyme and the redox mediator are distributed throughout the sensing layer.

44. The method of claim 39, further comprising contacting the sensing layer with a membrane that limits flux of the analyte to the sensing layer, wherein the membrane is disposed over the sensing layer.

45. The method of claim 44, wherein only the sensing layer comprises the thickener.

46. The method of claim 39, wherein the analyte sensor is a glucose sensor.

47. The method of claim 46, wherein the analyte-responsive enzyme comprises a glucose-responsive enzyme.

48. The method of claim 47, wherein the glucose-responsive enzyme comprises glucose oxidase.

49. The method of claim 46, wherein the redox mediator comprises a ruthenium-containing complex or an osmium-containing complex.

50. The method of claim 39, wherein the analyte sensor is an in vivo sensor.

51. The method of claim 39, wherein the analyte sensor is an in vitro sensor.

52-103. (canceled)
